SEQUENCE LISTING (89 sequences)

SEQ ID NO: 1 - DNA, 8 bp, Artificial Sequence
  Lx site spacer sequence
agtttata

SEQ ID NO: 2 - DNA, 8 bp, Artificial Sequence
  Lx site reverse orientation
tataaact

SEQ ID NO: 3 - DNA, 34 bp, Artificial Sequence
  Lx-LE mutant
taccgttcgtataagtttatatatacgaagttat

SEQ ID NO: 4 - DNA, 34 bp, Artificial Sequence
  Lx-LE mutant reverse orientation
ataacttcgtatatataaacttatacgaacggta

SEQ ID NO: 5 - DNA, 34 bp, Artificial Sequence
  Lx-RE mutant
ataacttcgtataagtttatatatacgaacggta

SEQ ID NO: 6 - DNA, 34 bp, Artificial Sequence
  Lx-RE mutant reverse orientation
taccgttcgtatatataaacttatacgaagttat

SEQ ID NO: 7 - protein, 343 aa, Bacteriophage P1
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 8 - RNA, 1029 nt, Bacteriophage P1
augagcaaccugcugaccgugcaccagaaccugcccgcccugcccguggacgccaccagcgacgaggugaggaagaaccugauggacauguucagggacaggcaggccuucagcgagcacaccuggaagaugcugcugagcgugugcaggagcugggccgccuggugcaagcugaacaacaggaagugguuccccgccgagcccgaggacgugagggacuaccugcuguaccugcaggccaggggccuggccgugaagaccauccagcagcaccugggccagcugaacaugcugcacaggaggagcggccugcccaggcccagcgacagcaacgccgugagccuggugaugaggaggaucaggaaggagaacguggacgccggcgagagggccaagcaggcccuggccuucgagaggaccgacuucgaccaggugaggagccugauggagaacagcgacaggugccaggacaucaggaaccuggccuuccugggcaucgccuacaacacccugcugaggaucgccgagaucgccaggaucagggugaaggacaucagcaggaccgacggcggcaggaugcugauccacaucggcaggaccaagacccuggugagcaccgccggcguggagaaggcccugagccugggcgugaccaagcugguggagagguggaucagcgugagcggcguggccgacgaccccaacaacuaccuguucugcagggugaggaagaacggcguggccgcccccagcgccaccagccagcugagcaccagggcccuggagggcaucuucgaggccacccacaggcugaucuacggcgccaaggacgacagcggccagagguaccuggccuggagcggccacagcgccagggugggcgccgccagggacauggccagggccggcgugagcauccccgagaucaugcaggccggcggcuggaccaacgugaacaucgugaugaacuacaucaggaaccuggacagcgagaccggcgccauggugaggcugcuggaggacggcgac

SEQ ID NO: 9 - protein, 7 aa, Simian virus 40
PKKKRKV

SEQ ID NO: 10 - protein, 16 aa, Simian virus 40
KRPAATKKAGQAKKKK

SEQ ID NO: 11 - protein, 25 aa, Caenorhabditis elegans
MSRRRKANPTKLSENAKKLAKEVEN

SEQ ID NO: 12 - protein, 9 aa, Homo sapiens
PAAKRVKLD

SEQ ID NO: 13 - protein, 9 aa, Escherichia coli
KLKIKRPVK

SEQ ID NO: 14 - DNA, 13 bp, Bacteriophage P1
ataacttcgtata

SEQ ID NO: 15 - DNA, 13 bp, Bacteriophage P1
tatacgaagttat

SEQ ID NO: 16 - DNA, 8 bp, Bacteriophage P1
atgtatgc

SEQ ID NO: 17 - DNA, 8 bp, Artificial Sequence
  L3 spacer sequence
aagtctcc

SEQ ID NO: 18 - DNA, 8 bp, Artificial Sequence
  L2 inverted spacer sequence
gcatacat

SEQ ID NO: 19 - DNA, 8 bp, Artificial Sequence
  LoxFas spacer sequence
tacctttc

SEQ ID NO: 20 - DNA, 8 bp, Artificial Sequence
  Lox511 spacer sequence
atgtatac

SEQ ID NO: 21 - DNA, 8 bp, Artificial Sequence
  Lox5171 spacer sequence
atgtgtac

SEQ ID NO: 22 - DNA, 8 bp, Artificial Sequence
  Lox2272 spacer sequence
aagtatcc

SEQ ID NO: 23 - DNA, 8 bp, Artificial Sequence
  Loxm2 spacer sequence
agaaacca

SEQ ID NO: 24 - DNA, 8 bp, Artificial Sequence
  Loxm3 spacer sequence
taatacca

SEQ ID NO: 25 - DNA, 8 bp, Artificial Sequence
  Loxm7 spacer sequence
agatagaa

SEQ ID NO: 26 - RNA, 22 nt, Artificial Sequence
  crRNA repeat
  misc_feature (11)..(11): a or g
  misc_feature (12)..(12): n is a, c, g, or u
  misc_feature (14)..(14): c or u
  misc_feature (15)..(15): n is a, c, g, or u
guuuuagagcunugnuguuuug

SEQ ID NO: 27 - DNA, 5 bp, Artificial Sequence
  FokI motiv
ggatg

SEQ ID NO: 28 - DNA, 608 bp, Human cytomegalovirus
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccgtttagtgaacgtcagatc

SEQ ID NO: 29 - DNA, 696 bp, Human cytomegalovirus
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccgtttagtgaacgtcagatctagctctgggagaggagcccagcactagaagtcggcggtgtttccattcggtgatcagcactgaacacagaggaagcttgccgccacc

SEQ ID NO: 30 - DNA, 2125 bp, Human cytomegalovirus
ctgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacggtttaaacgccgccacc

SEQ ID NO: 31 - DNA, 218 bp, Bos taurus
ttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 32 - DNA, 73 bp, Homo sapiens
caggataatatatggtagggttcatagccagagtaacctttttttttaatttttattttattttatttttgag

SEQ ID NO: 33 - DNA, 288 bp, Simian virus 40
agtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcg

SEQ ID NO: 34 - DNA, 129 bp, Simian virus 40
aacttgtttattgcagcttataatggttacaaataaagcaatagcatcacaaatttcacaaataaagcatttttttcaccattctagttgtggtttgtccaaactcatcaatgtatcttatcatgtctg

SEQ ID NO: 35 - DNA, 798 bp, Artificial Sequence
  green fluorescent protein encoding nucleic acid
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatctagatga

SEQ ID NO: 36 - DNA, 34 bp, Saccharomyces cerevisiae
gaagttcctattctctagaaagtataggaacttc

SEQ ID NO: 37 - DNA, 13 bp, Saccharomyces cerevisiae
gaagttcctattc

SEQ ID NO: 38 - DNA, 13 bp, Saccharomyces cerevisiae
gaataggaacttc

SEQ ID NO: 39 - DNA, 8 bp, Saccharomyces cerevisiae
tctagaaa

SEQ ID NO: 40 - DNA, 8 bp, Artificial Sequence
  F3 site spacer sequence
ttcaaata

SEQ ID NO: 41 - DNA, 8 bp, Artificial Sequence
  F5 site spacer sequence
ttcaaaag

SEQ ID NO: 42 - DNA, 32 bp, Artificial Sequence
  roxP site
taactttaaataatgccaattatttaaagtta

SEQ ID NO: 43 - DNA, 34 bp, Artificial Sequence
  LoxLTR site
acaacatcctattacaccctatatgccaacatgg

SEQ ID NO: 44 - DNA, 34 bp, Artificial Sequence
  LoxBTR site
aacccactgcttaagcctcaataaagcttgcctt

SEQ ID NO: 45 - DNA, 34 bp, Artificial Sequence
  LoxV site
tcaatttctgagaactgtcattctcggaaattga

SEQ ID NO: 46 - DNA, 34 bp, Artificial Sequence
  LoxFas site
acaacttcgtatatacctttctatacgaagttgt

SEQ ID NO: 47 - DNA, 8 bp, Artificial Sequence
  Missirlis-1 spacer sequence
gtatagta

SEQ ID NO: 48 - DNA, 8 bp, Artificial Sequence
  Missirlis-2 spacer sequence
ggctatag

SEQ ID NO: 49 - DNA, 8 bp, Artificial Sequence
  Loxm11 spacer sequence
tggtatcg

SEQ ID NO: 50 - DNA, 5 bp, Artificial Sequence
  LE inverted repeat mutant sequence
taccg

SEQ ID NO: 51 - DNA, 5 bp, Artificial Sequence
  RE inverted repeat mutant sequence
cggta

SEQ ID NO: 52 - DNA, 34 bp, Artificial Sequence
  Lox71 site
taccgttcgtatagcatacattatacgaagttat

SEQ ID NO: 53 - DNA, 34 bp, Artificial Sequence
  Lox66 site
ataacttcgtatagcatacattatacgaacggta

SEQ ID NO: 54 - DNA, 34 bp, Artificial Sequence
  LoxJTZ17 site
ataacttcgtatagcatacattatagcaatttat

SEQ ID NO: 55 - DNA, 34 bp, Artificial Sequence
  LoxKR1 site
ataacttcgtatagcatacattataccaactgtt

SEQ ID NO: 56 - DNA, 34 bp, Artificial Sequence
  LoxKR2  site
ataacttcgtatagcatacattataccaacttaa

SEQ ID NO: 57 - DNA, 34 bp, Artificial Sequence
  LoxKR3 site
ataacttcgtatagcatacattataccttgttat

SEQ ID NO: 58 - DNA, 34 bp, Artificial Sequence
  LoxKR4 site
ataacttcgtatagcatacattattgcaagttat

SEQ ID NO: 59 - DNA, 34 bp, Artificial Sequence
  LoxJT15 site
aattattcgtatagcatacattatacgaagttat

SEQ ID NO: 60 - DNA, 34 bp, Artificial Sequence
  FRT LE mutant
gaagttcatattctctagaaagtataggaacttc

SEQ ID NO: 61 - DNA, 34 bp, Artificial Sequence
  FRT RE mutant
gaagttcctattctctagaaagtatatgaacttc

SEQ ID NO: 62 - DNA, 201 bp, adeno-associated virus 2
gggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagctttttt

SEQ ID NO: 63 - DNA, 201 bp, adeno-associated virus 2
gggcactcttccgtggtctggtggataaattcgcaagggtatcatggcggacgaccgttgttcgaacaccggatccggccgtccgccgtgatccatgcggttaccgcccgcgtgtcgaacccaggtgtgcgacgtcagacaacgggggagcgctccttttggcttccttccaggcgcggcggctgctgcgctagctttttt

SEQ ID NO: 64 - DNA, 6 bp, adeno-associated virus 2
gggcac

SEQ ID NO: 65 - DNA, 6 bp, Homo sapiens
tttttt

SEQ ID NO: 66 - DNA, 157 bp, adeno-associated virus 2
aggagcgctcccccgttgtctgacgtcgcacacctgggttcgacacgcgggcggtaaccgcatggatcacggcggacggccggatccggtgttcgaacaacggtcgtccgccatgatacccttgcgaatttatccaccagaccacggaagagtgccc

SEQ ID NO: 67 - DNA, 313 bp, Artificial Sequence
  VA RNA inverted sequence with Lx-LE and Lx-RE
taccgttcgtataagtttatatatacgaagttatggacgaaacaccgggcacttttttcagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggtgttcgaacaacggtcgtccgccatgatacccttgcgaatttatccaccagaccacggaagagtgcccggtgtttcgtcctaccgttcgtatatataaacttatacgaagttat

SEQ ID NO: 68 - DNA, 12 bp, Artificial Sequence
  stuffer sequence
ggacgaaacacc

SEQ ID NO: 69 - DNA, 46 bp, Artificial Sequence
  Lx-LE site with stuffer sequence
taccgttcgtataagtttatatatacgaagttatggacgaaacacc

SEQ ID NO: 70 - DNA, 34 bp, Artificial Sequence
  L3 element with mutated left inverted repeat (L3-LE)
taccgttcgtataaagtctcctatacgaagttat

SEQ ID NO: 71 - DNA, 34 bp, Artificial Sequence
  L3 element with mutated right inverted repeat (L3-RE)
ataacttcgtataaagtctcctatacgaacggta

SEQ ID NO: 72 - DNA, 34 bp, Artificial Sequence
  L3 element with mutated left inverted repeat (LoxFas-LE)
taccgttcgtatatacctttctatacgaagttat

SEQ ID NO: 73 - DNA, 34 bp, Artificial Sequence
  L3 element with mutated right inverted repeat (LoxFas-RE)
ataacttcgtatatacctttctatacgaacggta

SEQ ID NO: 74 - DNA, 9 bp, Homo sapiens
tttatatat

SEQ ID NO: 75 - protein, 738 aa, Artificial Sequence
  VP1 Capsid
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 76 - protein, 736 aa, Artificial Sequence
  VP1 Capsid
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 77 - RNA, 4 nt, Human adenovirus type 2
ccgg

SEQ ID NO: 78 - RNA, 5 nt, Human adenovirus type 2
yccgg

SEQ ID NO: 79 - RNA, 10 nt, Artificial Sequence
  VA RNA A-box consensus sequence
  misc_feature (4)..(5): n is a, c, g, or u
rrynnarygg

SEQ ID NO: 80 - RNA, 9 nt, Artificial Sequence
  VA RNA B-box consensus sequence
  misc_feature (7)..(8): n is a, c, g, or u
gwucrannc

SEQ ID NO: 81 - DNA, 225 bp, Human adenovirus type 2
cgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagctttttt

SEQ ID NO: 82 - DNA, 225 bp, Human adenovirus type 2
cgtgcaaaaggagagcctgtaagcgggcactcttccgtggtctggtggataaattcgcaagggtatcatggcggacgaccgttgttcgaacaccggatccggccgtccgccgtgatccatgcggttaccgcccgcgtgtcgaacccaggtgtgcgacgtcagacaacgggggagcgctccttttggcttccttccaggcgcggcggctgctgcgctagctttttt

SEQ ID NO: 83 - DNA, 242 bp, Human adenovirus type 5
tcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagctttttt

SEQ ID NO: 84 - DNA, 466 bp, Human adenovirus type 5
tcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgctttt

SEQ ID NO: 85 - DNA, 52 bp, Artificial Sequence
  52bp minimal CMV promoter
gtaggcgtgtacggtgggaggtctatataagcagagctccgtttagtgaacg

SEQ ID NO: 86 - DNA, 417 bp, Artificial Sequence
  417 bp fragment of the SV40 early promoter including the transcription start (TS) region
atttcaggccatggtgctgcaacctctgaaagaggaacttggttaggttccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggactaggcttttgcaaaaagcta

SEQ ID NO: 87 - protein, 236 aa, Artificial Sequence
  mCherry
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 88 - protein, 239 aa, Artificial Sequence
  EGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 89 - DNA, 34 bp, Artificial Sequence
  FRT-LE-RE
gaagttcatattctctagaaagtatatgaacttc

(19) United States
(12) Patent Application Publication
(10) Pub. No.: US 2022/0154223 A1
(43) Pub. Date: May 19, 2022

(54) NUCLEIC ACID CONSTRUCTS FOR SIMULTANEOUS GENE ACTIVATION

(71) Applicant: Hoffmann-La Roche Inc., Little Falls, NJ (US)

(72) Inventors: Simon Auslaender, Wolfratshausen (DE); Ulrich Goepfert, Penzberg (DE)

(73) Assignee: Hoffmann-La Roche Inc., Little Falls, NJ (US)

(21) Appl. No.: 17/500,774

(22) Filed: Oct. 13, 2021

(30) Foreign Application Priority Data
Oct. 15, 2020 (EP) 20202009.5

(51) Int. Cl. C12N 15/90; C12N 15/85

(52) U.S. Cl. CPC C12N 15/907; C12N 2800/30; C12N 15/85

(57) ABSTRACT

Herein are reported novel DNA constructs and methods using the same. The current invention uses a deliberate arrangement of non-productive/inactive promoters and genes on coding and template strands of DNA molecules, which are converted into their active form by the interaction with a site-specific recombinase. In more detail, the DNA element according to the current invention is non-functional with respect to the expression of the contained first and second genes. By being non-functional with respect to the expression of the first and second gene, the DNA element according to the invention can be integrated into genome of a cell without the risk that the comprised structural genes are expressed already directly after the integration. The genes are only expressed once a recombinase recognizing and functional with the recombination recognition sequences of the DNA element is activated or introduced into the cell. Thereby, a recombinase mediated cassette inversion (RMCI) between the first and second mutated recombinase recognition sequences in the genomically integrated DNA element of the invention is initiated. The RMCI results in an inversion of that part of the DNA element according to the invention that is located between the two mutant recombinase recognition sequences. Thereby the first promoter becomes operably linked to the first gene and the second promoter becomes operably linked to the second gene. Only thereafter, the first and second genes are transcribed and the respective encoded proteins are expressed. Thus, the DNA element according to the current invention is especially useful in the simultaneous activation of two genes within a cell.

### CROSS-REFERENCE TO RELATED APPLICATIONS

This application claims priority to European Patent Application No. 20202009.5 filed Oct. 15, 2020, all of which are incorporated by reference in its entirety.

### SEQUENCE LISTING

This application contains a Sequence Listing which has been submitted electronically in ASCII format and is hereby incorporated by reference in its entirety. Said ASCII copy, created on Sep. 30, 2021, is named P36312-US_Sequence_Listing.txt and is 46,400 bytes in size.

### FIELD OF INVENTION

Herein are reported novel DNA constructs and methods of using the same. With the novel DNA constructs according to the current invention the transcription of at least two genes can be activated simultaneously using site-specific recombinase technology. The current invention uses a deliberate inactive arrangement of promoters and gene elements on coding and template strands of DNA molecules, which are converted into their active form by the interaction with a site-specific recombinase. Also reported herein is a novel VA RNA element with exchanged promoter and incorporated LoxP site.

### BACKGROUND OF THE INVENTION

Gene therapy refers broadly to the therapeutic administration of genetic material to modify gene expression of living cells and thereby alter their biological properties. After decades of research, gene therapies have progressed to the market and are expected to become increasingly important. In general, gene therapy can be divided into either in vivo or ex vivo approaches.

Today, most in vivo therapies rely on DNA delivery with recombinant adeno-associated viral (rAAV) vectors. An AAV is a small, naturally occurring, non-pathogenic parvovirus, which is composed of a non-enveloped icosahedral capsid. It contains a linear, single stranded DNA genome of approximately 4.7 kb. The genome of wild-type AAV vectors carries two genes, rep and cap, which are flanked by inverted terminal repeats (ITRs). ITRs are necessary in cis for viral replication and packaging. The rep gene encodes for four different proteins, whose expression is driven by two alternative promoters, P5 and P19. Additionally different forms are generated by alternative splicing. The Rep proteins have multiple functions, such as, e.g., DNA binding, endonuclease and helicase activity. They play a role in gene regulation, site-specific integration, excision, replication and packaging. The cap gene codes for three capsid proteins and one assembly-activating protein. Differential expression of these proteins is accomplished by alternative splicing and alternative start codon usage and driven by a single promoter, P40, which is located in the coding region of the rep gene.

In engineered, therapeutic rAAV vectors, the viral genes are replaced with a transgene expression cassette, which remains flanked by the viral ITRs, but encodes a gene of interest under the control of a promoter of choice. Unlike the wild-type virus, the engineered rAAV vector does not undergo site-specific integration into the host genome, remaining predominantly episomal in the nucleus of transduced cells.

An AAV is not replication competent by itself but requires the function of helper genes. These are provided in nature by co-infected helper viruses, such as, e.g., adenovirus or herpes simplex virus. For instance, five adenoviral genes, i.e. E1A, E1B, E2A, E4 and VA, are known to be essential for AAV replication. In contrast to the other helper genes, which code for proteins, VA is a small RNA gene.

For the production of rAAV vectors, DNA carrying the transgene flanked by ITRs is introduced into a packaging host cell line, which also comprise rep and cap genes as well as the required helper genes. There are many ways of introducing these three groups of DNA elements into cells and ways of combining them on different DNA plasmids (see, e.g., Robert, M. A., et al. Biotechnol. J. 12 (2017) 1600193).

Two general production methods are widely used. In the triple transfection method, HEK293 cells, which already express adenovirus E1A and E1B, are transiently co-transfected with an adenovirus helper plasmid (pHELPER) carrying E2A, E4 and VA, a plasmid comprising rep/cap and a plasmid comprising the rAAV-transgene. Alternatively, rep/cap and viral helper genes can be combined on one larger plasmid (dual transfection method). The second method encompasses the infection of insect cells (Sf9) with two baculoviruses, one carrying the rAAV genome and the other carrying rep and cap. In this systems helper functions are provided by the baculovirus plasmid itself. In the same way, herpes simplex virus is used in combination with HEK293 cells or BHK cells. More recently Mietzsch et al. (Hum. Gene Ther. 25 (2014) 212-222; Hum. Gene Ther. Methods 28 (2017) 15-22) engineered Sf9 cells with rep and cap stably integrated into the genome. With these cells a single baculovirus carrying the rAAV transgene is sufficient to produce rAAV vectors. Clark et al. (Hum. Gene Ther. 6 (1995) 1329-1341) generated a HeLa cell line with rep/cap genes and a rAAV transgene integrated in its genome. By transfecting the cells with wild-type adenovirus, rAAV vector production is induced and mixed stocks of rAAV vectors and adenovirus are produced.

No mammalian cell line with helper genes stably integrated into its genome have been described so far. Expression of rep as well as viral helper genes is toxic to cells and needs to be tightly controlled (see, e.g., Qiao, C., et al., J. Virol. 76 (2002) 1904-1913).

For rep genes such a control has been accomplished by introducing an intron into the rep gene that contains a polyadenylation sites flanked by LoxP sites. After introducing Cre-recombinase with the help of a recombinant adenovirus, the polyadenylation sites are removed and the intron is spliced out (see, e.g., Yuan, Z., et al., Hum. Gene Ther. 22 (2011) 613-624; Qiao, C., et al., supra).

Podhajska, A. J., et al. (Gene 40 (1985) 163-168) reported a prototype of gene-expression plasmids with three novel properties: (i) its “OFF phase” is absolute in all common hosts because the expression promoter is facing away from the studied gene and is blocked by a strong terminator; (ii) the “ON phase” is attained by the rapid and efficient inversion of the promoter; (iii) only a short heat pulse or exposure to other inducing agents is required to initiate this two-stage process.

WO 97/9441 (EP 0 850 313 B1) reported a method for producing recombinant adeno-associated virus (AAV), said method comprising the steps of: (1) culturing a composition comprising cells which have been transiently transfected with: (a) an AAV helper plasmid comprising nucleic acids encoding AAV rep and cap proteins; (b) an adenoviral helper plasmid comprising essential adenovirus helper genes, said essential adenovirus helper genes present in said plasmid being selected from the group consisting of E1A, E1B, E2A, E4, E4ORF6, E4ORF6/7, VA RNA and combinations thereof; and (c) an AAV plasmid comprising first and second AAV inverted terminal repeats (ITRs), wherein said first and second AAV ITRs flank a DNA encoding a polypeptide of interest, said DNA being operably linked to a promoter DNA; in the absence of adenovirus particles; and (2) purifying recombinant AAV produced therefrom.

JP 10-33175 A reported a gene sequence in which a stuffer sequence flanked by two recombinase recognition sequences has been inserted into an adeno-associated virus genome sequence, wherein the gene sequence is characterized in that the insertion site of the recombinase recognition sequence is between a promoter P5 and a translation initiation codon of a rep78/68 gene, and the stuffer sequence contains at least one detectable gene marker and polyA signal in the same direction as the promoter P5 and the rep78/68 gene.

WO 98/24918 (EP 0 942 999 B1; U.S. Pat. No. 6,303,302 B1) reported a gene-trapping construct, containing a first reporter gene, which after activation can activate a second reporter gene, wherein the first reporter gene codes for a recombinase, the second reporter gene codes for a protein factor and the second reporter gene is activated thereby that the recombinase deletes a DNA fragment located before the second reporter gene and in that way places the second reporter gene downstream from a promoter under its control.

WO 98/27207 reported a polynucleotide comprising a recombinase-activatable adeno-associated virus (AAV) packaging cassette comprising the following components in the relative order listed from upstream to downstream: (i) a first site-specific recombination (ssr) site; (ii) an ssr-intervening sequence; and (iii) a second site-specific recombination (ssr) site; wherein the cassette comprises a promoter and an AAV packaging gene selected from the group consisting of an AAV rep gene and an AAV cap gene, wherein said promoter is located either within the ssr-intervening sequence or upstream of the first ssr site and said AAV packaging gene is located either downstream of the second ssr site or within the ssr-intervening sequence, and wherein said promoter is activatably linked to said AAV packaging gene.

WO 98/10086 (U.S. Pat. No. 6,274,354 B1) reported methods for efficient production of recombinant AAV. In one aspect, three plasmids are introduced into a host cell. A first plasmid directs expression of Cre-recombinase, a second plasmid contains a promoter, a spacer sequence flanked by LoxP sites and rep/cap, and a third plasmid contains a minigene containing a transgene and regulatory sequences flanked by AAV ITRs. In another aspect, the host cell stably or inducibly expresses Cre-recombinase and two plasmids carrying the other elements of the system are introduced into the host cell.

WO 98/27217 (EP 0 953 647 B1) reported a DNA construction for regulating the expression of a virus structural protein gene by using a recombinase and its recognition sequence, wherein a promoter, the recombinase recognition sequence, a drug resistance gene, a polyA addition signal, the recombinase recognition sequence, the virus structural protein gene and a polyA addition signal are arranged in this order.

WO 2001/36615 (EP 1 230 354 B1) reported a permanent amniocytic cell line comprising at least one nucleic acid which brings about expression of the gene products of the adenovirus E1A and E1B regions.

WO 2001/66774 reported a system to control the expression of a gene of interest comprising a first DNA sequence comprising a gene of interest linked in functional relation to a promoter, and a second DNA sequence comprising a second gene that encodes a polypeptide having a recombinant activity specific for target DNA sequences, and two of said target DNA sequences flanking one of the said two DNA sequences, characterized in that said second DNA sequence is located between said promoter and said gene of interest.

Silver, D. P. and Livingstone, D. M. reported that continuous expression of the Cre-recombinase in cultured cells lacking exogenous LoxP sites caused decreased growth, cytopathic effects, and chromosomal aberrations. A self-excising retroviral vector that incorporates a negative feedback loop to limit the duration and intensity of Cre-recombinase expression avoided measurable toxicity and retained the ability to excise a target sequence flanked by LoxP sites (Mol. Cell 8 (2001) 233-243).

Siegel, R. W., et al. outlined that given the growing importance of the Cre/LoxP system for the elucidation of gene function, more elaborate schemes to activate or deactivate genes, as well as allowing selectable markers to be recycled for subsequent re-use require the availability of sets of non-compatible LoxP sites. Integrating multiple non-compatible LoxP sites into a genome at defined locations allows the subsequent Cre-recombinase-mediated introduction of a transgene construct to different chromosomal locations by simply specifying the corresponding LoxP sites on the targeting vector (FEBS Lett. 499 (2001) 147-153).

WO 2002/8409 (EP 1 309 709 A2, U.S. Pat. No. 7,972,857) reported a method of obtaining site-specific replacement of a DNA of interest in a mammalian cell, comprising a) providing a mammalian cell that comprises a receptor construct, wherein the receptor construct comprises a receptor polynucleotide to be replaced, the receptor polynucleotide being flanked by two or more copies of an irreversible recombination site (IRS); b) introducing into the cell a donor construct that comprises a donor polynucleotide to replace the receptor polynucleotide, the donor polynucleotide being flanked by two or more of a complementary irreversible recombination site (CIRS); and c) contacting the receptor construct and the donor construct with an irreversible recombinase polypeptide; wherein the irreversible recombinase catalyzes recombination between the IRS and the CIRS and replacement of the receptor polynucleotide with the donor polynucleotide, thereby forming a replacement construct.

WO 2002/40685 (U.S. Pat. No. 7,449,179 B2) reported a method of preparing gene-trapping libraries, and gene targeted cells for conditional inactivation of genes. A plasmid having a mutational element cassette and a gene trap cassette, each cassette having site-specific recombination sequences were provided. The mutational element cassette comprised a first site-specific recombination sequence and a DNA comprising a mutational sequence comprising a splice acceptor sequence linked to a first marker gene linked to a polyadenylation sequence and a second site-specific recombination sequence. The gene trap cassette comprised a first site specific recombination sequence and a DNA comprising a first gene trap element comprising a promoter operably linked to a second marker gene operably linked to a splice donor sequence and a second gene trap cassette comprising a promoter linked to a unique sequence not present in the genome of a selected host cell.

WO 2002/88353 (EP 1 383 891 B1) reported an isolated DNA molecule comprising at least a sequence A flanked by at least site specific recombinase targeting sequences (SSRTS) L1, and at least a sequence B flanked by at least site specific recombinase targeting sequences (SSRTS) L2, said sequences A and B being transcribed and translated sequences in an opposite orientation, said SSRTS L1 and SSRTS L2 being unable to recombine with one another, and wherein sequences L1 are in an opposite orientation, sequences L2 are in an opposite orientation, the order of SSRTS sequences in said DNA molecule is 5′-L1-L2-L1-L2-3′, and the recombinase specific of said SSRTS L1 and the recombinase specific of said SSRTS L2 are the same.

Mlynarova, L., et al. reported that in Escherichia coli both Lox511 and Lox2272 sites become highly promiscuous with respect to LoxP when in the presence of Cre-recombinase one of the recombination partners is present in a larger stretch of an inverted repeat of non-lox DNA (Gene 296 (2002) 129-137).

Langer, S. J., et al. reported that use of LoxP sites with complementary mutant arms (Lox66 and Lox71) allowed efficient recombination in trans generating a wild-type LoxP site and a defective site with doubly mutant arms (Nucl. Acids Res. 30 (2002) 3067-3077). Because the doubly mutant LoxP site is no longer an efficient substrate for the recombinase, insertion is favored and the reaction is driven in one direction.

Tronche, F., et al. reported the use of site-specific recombinases in mice (FEBS Lett 529 (2002) 116-121). They outlined that in mice, the Cre-LoxP system was initially used to switch on gene expression in a given cell population. Two distinct transgenic mouse lines were generated. The first carries a silent transgene, which is spaced from a promoter by a ‘stop cassette’. The stop cassette prevents transcription of the transgene because it contains either a strong polyadenylation signal and/or a splice donor sequence or it disrupts the ORF of the silent gene. The second carries a transgene that drives the expression of Cre-recombinase in a cell type-specific, i.e. tissue-specific, way. In every Cre-recombinase expressing cell, the stop cassette will be excised enabling the expression of the desired transgene exclusively in those cells. According to Tronche et al., it is essential that the insertion of the LoxP sites does not interfere with the normal expression of the gene. Ideally, they should be placed in introns or non-transcribed regions, avoiding the disruption of regulatory regions. However, in several cases a LoxP site was inserted in transcribed but untranslated regions without negative effects. Tronche et al. further outline that a decrease in cell proliferation as well as an increase in apoptosis in cells expressing high levels of Cre-recombinase has been observed. This is associated with the accumulation of Cre-recombinase expressing cells in the G2/M phase of the cell cycle, chromosomal rearrangement and the appearance of micronuclei. These aberrations could be due to the action of Cre-recombinase on cryptic target sites that exist in the genome.

WO 2003/84977 reported a gene expression control method that employs a transcription termination sequence positioned within an intron. The transcription termination sequence is disruptable by the addition of a trans-acting factor. For example, in a “dual splicing switch”, the transcription termination sequence is flanked by recombination sites and can be excised by a recombinase. The Cre/LoxP recombination system may be used for this purpose.

Thomson, J. G., et al. reported that the insertion reaction in the Cre/LoxP system is more difficult to control since the excision event is kinetically favored. Comparison of 50 mutant LoxP sites combinations to the native LoxP site revealed that mutations to the inner 6 bp of the Cre-recombinase binding domain severely inhibited recombination, while those in the outer 8 bps were more tolerated (Genesis 36 (2003) 162-167).

WO 2004/29219 reported vectors and methods for controlling the temporal and spatial expression of a shRNA construct in cells and organisms. Such vectors may be retroviral vectors, such as lentiviral vectors. In preferred embodiments, expression of a shRNA is regulated by an RNA polymerase III promoter; such promoters are known to produce efficient silencing. While essentially any polIII promoter may be used, desirable examples include the human U6 snRNA promoter, the mouse U6 snRNA promoter, the human and mouse Hl RNA promoter and the human tRNA-val promoter.

Mizukami, H., et al., reported the separate control of rep and cap expression using mutant and wild-type LoxP sequences and improved packaging system for adeno-associated virus vector production. They have developed an inducible expression system for both Rep and Cap proteins by using two separate plasmids, one with mutant and the other with wild-type LoxP sequences, the expression of two different proteins can be induced simultaneously by Cre-recombinase (Mol. Biotechnol. 27 (2004) 1-14). To effect recombination a Cre-recombinase-expressing adenovirus plasmid was applied to the culture. To control rep and cap expression, a stuffer sequence is flanked by two LoxP (wild-type or mutant) sequences. In the presence of Cre-recombinase, the stuffer sequences are removed and the cap and rep genes are expressed.

Chatterjee, P. K., et al. reported that the differences between the results obtained in vivo and those reported earlier might be related to the transient versus constitutively expressed Cre-recombinase protein available for the recombination. LoxP site promiscuity does appear to increase with the level and persistence of Cre-recombinase protein (Nucl. Acids Res. 32 (2004) 5668-5676).

Ventura, A., et al. reported Cre-lox-regulated conditional RNA interference from transgenes (Proc. Natl. Acad. Sci. USA 101 (2004) 10380-10385). The authors have generated two lentiviral vectors for conditional, Cre-lox-regulated RNA interference. One vector allows for conditional activation, whereas the other permits conditional inactivation of short hairpin RNA (shRNA) expression. The former is based on a strategy in which the mouse U6 promoter has been modified by including a hybrid between a LoxP site and a TATA box.

US 2006/110390 reported adenovirus expression vectors AdCMV-Ku70 and AdCMV-Ku80, which are based on the Cre-recombinase-dependent luciferase expression plasmid, AdCUL consisting of oppositely oriented mutant LoxP sites, Lox71 and Lox66, flanking an anti-sense firefly luciferase reporter gene downstream of the cytomegalovirus immediate early promoter (CMV). Cre-recombinase-mediated recombination between Lox71 and Lox66 inverts the floxed cassette into the sense orientation, resulting in luciferase gene expression.

US 2006/143737 (U.S. Pat. No. 7,267,979 B2) reported a construct for recombinase inversion or excision yielding double-stranded target sequence RNA, which thereby functions to trigger endogenous gene silencing mechanisms.

WO 2006/99615 reported the application of Cre-recombinase and half-mutant LoxP sites with incompatible spacers to uni-directionally exchange modified targeting genes into the fiber region of adenoviral vectors.

Missirlis, P. I., et al. (BMC Genomics 7 (2006) A13) reported a high-throughput screen identifying sequence and promiscuity characteristics of the LoxP spacer region in Cre-recombinase-mediated recombination. They outlined that given that spacer and inverted repeat mutants have been used together successfully, it may be possible to introduce numerous DNA segments into a given target molecule, chromosome or genome if a sufficient number of non-promiscuous LE/RE-spacer mutants can be identified. However, serializing RMCE or insertional recombination via inverted repeats has been limited by the small number of stable, non-promiscuous LoxP sites identified to date.

WO 2015/068411 reported an AAV-LoxP-plasmid comprising a nucleotide sequence encoding the target protein that is located between Lox71 and LoxJTZ17 in opposite direction to the orientation of the promoter, which usually does not express the protein of interest.

WO 2011/100250 reported a targeting plasmid for in vivo gene regulation in a eukaryotic cell, wherein the targeting plasmid introduces the LoxP-FRT-Neo STOP-FRT-tetO-LoxP cassette at a particular locus in the genome.

Kawabe, Y., et al. reported a gene integration system for antibody production using recombinant Chinese hamster ovary (CHO) cells (Cytotechnol. 64 (2012) 267-279). An exchange cassette flanked by wild-type and mutated LoxP sites was integrated into the chromosome of CHO cells for the establishment of recipient founder cells. Then, a donor plasmid including a marker-antibody-expression cassette flanked by a compatible pair of LoxP sites and also comprising an internal not-paired LoxP site between the expression cassette for the selection marker and the expression cassette of the antibody was prepared. The donor plasmid and a Cre-recombinase expression plasmid were co-transfected into the founder CHO cells to give rise to RMCE in the CHO genome, resulting in site-specific integration of the antibody gene restoring the original wild-type LoxP site and generating an inactive double-mutated LoxP site that no longer participates in RMCE. The RMCE procedure was repeated to increase the copy numbers of the integrated gene whereby in each step the expression cassette for the selection marker present in the cell was excised and removed.

Niesner, B. and Maheshri, N. reported that by inserting promoters flanked by inverted LoxP sites in front of a gene of interest the expression can randomly be altered by Cre-recombinase mediated flipping of the promoter. This is like a merry-go-round process constantly flipping the orientation of the promoter. Termination of the process is effected by termination of Cre-recombinase expression. However, while Cre-recombinase is highly efficient, multiple inversion events may result in irreversible loss of the floxed promoter or recombination with other genomic regions leading to a large-scale rearrangement (Biotechnol. Bioeng. 110 (2013) 2677-2686).

WO 2013/014294 reported the replacement of a first gene with a selection marker, for example the chloramphenicol acetyl transferase antibiotic marker, by homologous recombination, whereby the marker can be removed due to the presence of LoxP sites at both ends of the marker. In the setup used, two modified LoxP sites are used (Lox66 and Lox71), each with a different mutation. After recombination by Cre-recombinase, a Lox72 site is left (Lambert, J. M., et al., Appl. Environ. Microbiol. 73 (2007) 1126-1135), which has now two mutations instead of one, and can no longer be recognized by the Cre-recombinase.

US 2013/58871 reported the generation of a Cre-recombinase-mediated switchable inversion plasmid by using two mutant LoxP sites (Lox66 and Lox71) oriented in a head-to-head position. When Cre-recombinase is present, the gene flanked by the two mutant LoxP sites is inverted, forming one LoxP and one double-mutated LoxP site. Because the double-mutated LoxP site shows very low affinity for Cre-recombinase, the favorable one-step inversion is nearly irreversible, allowing the gene to be stably switched ‘on’ and ‘off’ as desired. Leakiness of expression in the absence of Cre-recombinase was minimized by eliminating sequences containing false TATA boxes and start codons at the sides of the floxed gene.

WO 2015/38958 reported a cap-in-cis rAAV genome, wherein a ubiquitin C promoter fragment is used to drive expression of an mCherry reporter followed by a synthetic polyA sequence; an AAV capsid gene, controlled by rep regulatory sequences, is followed by a Lox71- and Lox66-flanked SV40 late polyA signal; the Lox66 site is inverted relative to Lox71 site; in this configuration, Cre-recombinase mediates the inversion of the sequence flanked by the mutant LoxP sites; after the inversion, incompatible, double mutant Lox72 and a LoxP site are generated, reducing the efficiency of inversion back to the original state.

WO 2015/68411 reported a virus AAV-LoxP-WGA, a nucleotide sequence encoding the target protein, which is in the opposite direction to the orientation of the promoter. This construct usually does not express the protein of interest. When the nucleotide sequence encoding the protein of interest between said site-specific recombinase recognition sequences is inverted in direction the target protein is expressed.

Arguello, T. and Moraes, C. T. reported that Cre-recombinase activity is inhibited in vivo but not ex vivo by a mutation in the asymmetric spacer region of the distal LoxP site.

WO 2016/57800 reported a TGG or DRG promoter operably linked to a Cre-recombinase and a LOX-stop-LOX inducible RNA polymerase III promoter operably linked to an inhibitory RNA. In vivo, the authors have found that a single T to C mutation at position 4 of the central spacer region in the distal (3′) LoxP site completely inhibited the recombination reaction in two conditional mouse models.

WO 2017/100671 reported Cre-recombinase-dependent recovery of AAV capsid sequences from transduced target cells. In the rAAV-Cap-in-cis-lox rAAV genome, the polyadenylation (pA) sequence flanked by the Lox71 and Lox66 sites is inverted by Cre-recombinase.

WO 2017/189683 reported genetic constructs comprising genetic perturbation cassettes and methods of using such to assess the timing and order of gene expression.

WO 2018/96356 reported a method for generating an allele for conditional gene knock-out in a cell comprising a target gene, the method comprising: introducing an artificial intron sequence into an exon of the target gene, the artificial intron sequence comprising: a splice donor sequence; a first nuclease or recombinase site; a branch point sequence; a second nuclease or recombinase site; a splice acceptor sequence; and a stop codon positioned 5′ to or within the first nuclease or recombinase site, wherein for inactivation of the introduced intron, the method includes the step of introducing or activating a recombinase or nuclease in the cell thereby excising or disrupting the branch point and abrogating splicing of the artificial intron sequence.

WO 2018/229276 reported a conditional knock-in cassette which is a double stranded DNA molecule comprising a sequence A, a sequence B, a first pair RTS1 and RTS1′ and a second pair RTS2 and RTS2′ of recombinase target sites (RTS), wherein (i) RTS of the first pair and RTS of the second pair are unable to recombine together, and (ii) RTS1 and RTS1′ are in an opposite orientation, and (iii) RTS2 and RTS2′ are in an opposite orientation, and (iv) sequences A and B and RTS are in the following order from 5′ to 3′: RTS1, sequence A, RTS2, sequence B, RTS1′ and RTS2′, and (v) sequences A and B each comprises at least one coding sequence and said coding sequences are on different DNA strands, and (vi) the amino acid sequence encoded by sequence A has at least 90% sequence identity to the amino acid sequence encoded by sequence B, and (vii) the coding strand of sequence A and the non-coding strand of sequence B are unable to hybridize.

WO 2019/46069 reported selective recovery of the AAV cap gene by flanking the cap gene with a pair of LoxP sites and development of cell-type-specificity of Cre-recombinase expression. AAV infection of a Cre-recombinase expressing cell followed by second strand AAV genome synthesis led to the inversion of the floxed cap. Mutant LoxP sites Lox66 and Lox71 were utilized to drive the equilibrium of Cre-recombinase-mediated recombination towards unidirectional inversion. The LoxP sites were initially inserted in the 3′ UTR of cap, where they flanked short stuffer sequences containing the target sequence for Cre-recombinase-dependent recovery.

Fischer, K. B., et al. reported sources of off-target expression from recombinase-dependent AAV vectors and mitigation with cross-over insensitive ATG-out vectors (Proc. Natl. Acad. Sci. USA 116 (2019) 27001-27010). Recombinase-dependent adeno-associated viruses (AAVs) allow for targeting of specific regions and expression of different transgenes without the comparatively cumbersome process of transgenic mouse line production. While recombinase-dependent AAV designs using the lox-STOP-lox and FRT-STOP-FRT system have been used, double-inverted open reading frame (ORF) (DIO) and flip/excision (FLEX) constructs, effectively identical in their design, have gained the most widespread use for their limited size and purported less leaky nature when using strong promoters. Briefly, the DIO and FLEX designs use two pairs of orthogonal recognition sites in an overlapping antiparallel orientation around the desired transgene that is, with respect to the rest of the expression cassette, inverted and, thus, transcriptionally repressed. When exposed to the appropriate recombinase, the transgene ORF is reverted and locked in-sense with the promoter and 3′-untranslated region (UTR), driving expression. In the inverted ORF, sometimes called “ATG-out” or “split-transgene”, the Kozak sequence and the initiating codon of the transgene are placed outside the first set of recombinase recognition sites, leaving the transgene ORF to be reconstituted only following recombination. By independently disrupting spontaneous inversion and the transgene ORF, the authors show that both must be disrupted to fully abrogate leak. Further, while leak expression from an intact ORF is only detectable in highly sensitive systems, spontaneous inversions can drive low but detectable levels of expression of fluorescent proteins. Finally, the authors show that the use of mutant recombinase recognition sites with reduced homology in AAVs utilizing an ATG-out transgene design, which the authors dub CIAO (crossover insensitive ATG-out), greatly reduces leak expression in the mouse brain of a recombinase reporter mouse.

Transient transfection methods require large quantities of plasmid DNA that need to be produced by large-scale fermentation and DNA purification. More importantly, the scalability of DNA complexation with transfection reagents is limited. Scalability of electroporation is limited, too. In addition, transient transfection of cells is poorly reproducible.

Systems that rely on herpes simplex or adenovirus transduction have the intrinsic risk that rAAV preparations are contaminated with replication competent helper virus.

Baculovirus based systems have three major disadvantages: firstly, due to the large size of the baculovirus genome, which is in the range of 100 kb, tedious techniques need to be applied to generate and prepare recombinant virus DNA. Secondly, highly concentrated recombinant virus stocks need to be prepared prior to the actual production campaign. Finally, rAAV derived from baculovirus-based systems can easily suffer from altered capsid composition and lower potency. Therefore, additional effort are necessary to adjust the expression ratio of the different capsid proteins (Kondratov, O., et al., Mol. Ther. 25 (2017) 2661-2675).

Ojala, D. S., et al. reported that the in vivo selection of a computationally designed SCHEMA AAV library yields a novel variant for infection of adult neural stem cells in the SVZ (Mol. Thera. 26 (2018) 304-319.

WO 2020/78953 reported an adeno-associated virus (AAV) vector producer cell comprising nucleic acid sequences encoding AAV rep and cap genes, helper virus genes, and a DNA genome of the AAV vector; the AAV rep gene comprising an intron, the intron comprising a transcription termination sequence with a first recombination site located upstream and a second recombination site located downstream of the transcription termination sequence; and the nucleic acid sequences all integrated together at a single locus within the AAV vector producer cell genome. The invention also relates to methods for producing the AAV vector producer cell lines.

WO 2018/150271 reported a mammalian cell comprising at least four distinct recombination target sites (RTS), an adenoviral (Ad) gene comprising E1A, E1B or a combination thereof, and a promoter operatively linked to the Ad gene, wherein the RTS, the Ad gene, and the promoter are chromosomally-integrated; methods for using the cell for generating a recombinant adeno-associated vims (rAAV) producer host cell; and methods for using the AAV producer host cell to produce, package and purify rAAV.

Mingqi, X., et al. reported about mammalian designer cells—engineering principles and biomedical applications (Biotechnol. J. 10 (2015) 1005-1018.

Thus, there is a need for functional genomics tools, with which the number of transgenic DNA segments that can be selectively addressed in a genomic sequence is increased.

### SUMMARY OF THE INVENTION

Herein are reported novel deoxyribonucleic acids and methods using the same. The novel deoxyribonucleic acids according to the current invention are useful in the simultaneous activation of the expression of at least two open reading frames/genes by site-specific recombinase technology. The current invention uses a deliberate inactive arrangement of promoters and open reading frames/gene elements on the coding strand (the (+) strand, the positively oriented strand) and on the template strand (the (−) strand, the negatively oriented strand) of deoxyribonucleic (DNA) molecules, which require for transcriptional activation, i.e. operable linkage of promoter and coding sequence allowing transcription of said coding sequence, inversion by the interaction with a site-specific recombinase.

Also an aspect of the current invention is a recombinase-activatable packaging cell line for rAAV particle production, wherein rep/cap genes as well as adenoviral helper genes are (stably) integrated into the genome and wherein at least one of them, in one preferred embodiment at least two of them, is comprised in a deoxyribonucleic acid according to the current invention and can thereby be transcriptionally activated by the interaction with a site-specific recombinase. In certain embodiments, the transcriptional activation of one or more adenoviral helper genes is accomplished by recombinase-mediated open reading frame/gene inversion (RMCI). For example, after its activation the adenoviral helper protein E1A activates the transcription of the rep gene from the autologous P5 promoter, which in turn activates transcription of the cap gene. In certain embodiments, rep/cap gene transcription is activated using recombinase-mediated open reading frame/gene inversion in a deoxyribonucleic acid according to the current invention, in cells in which the adenoviral E1A protein is constitutively expressed, as for instance in HEK cells, or a heterologous promoter is used to drive rep and/or cap gene transcription. In a certain embodiment, the recombinase is Cre-recombinase form bacteriophage P1.

Cre-recombinase expression is, in certain embodiments, induced by transient transfection of small amounts of a Cre-recombinase encoding nucleic acid. It has been found that efficient recombination can be accomplished with as little as 10% of the amount of plasmid DNA that is usually used for transient virus production. Even lower amounts of nucleic acid are sufficient if Cre-recombinase encoding mRNA is used. In certain embodiments, a Cre-recombinase encoding nucleic acid is integrated into the packaging cell line's genome and operably linked to an inducible promoter, such as, e.g., a Tet-inducible promoter. In one preferred embodiment, the rAAV genome, comprising the ITRs and the transgene, is also integrated in the packaging cell line's genome. Thereby a packaging cell line is turned into a rAAV vector and particle producing cell line. Likewise, in certain embodiments, the rAAV genome is introduced transiently.

After recombination, the cells of the producing cell line are genetically uniform and express all genes that are required for rAAV replication and packaging in the correct stoichiometry (in contrast thereto, in triple or dual transfection methods some cells may receive suboptimal doses of one or the other plasmids/genes). Thus, without being bound by this theory, a stable rAAV vector/particle packaging or producing cell line may result in higher product quality compared to transient packaging or producing cells. In addition, induction of rAAV vector or particle production by transfection with a Cre-recombinase encoding nucleic acid instead of a helper virus provides for improved safety of the produced rAAV vector/particle.

A further aspect of the invention is a novel adenoviral VA RNA gene. The adenoviral VA RNA gene according to the current invention enables Cre-recombinase mediated gene activation by inversion. In the adenoviral VA RNA according to the current invention, the adenoviral VA RNA gene can be driven by any promoter with a precise transcription start site together with a LoxP site introduced into the non-coding, i.e. regulatory, elements of the adenoviral VA RNA.

A further aspect of the current invention is the novel LoxP site (spacer sequence) AGTTTATA (SEQ ID NO: 01 (forward orientation); SEQ ID NO: 02 (reverse orientation)). This spacer sequence is termed Lx herein. It can be combined with any known left and right repeat sequences.

In certain embodiments, the Lx spacer sequence is combined with a mutated left inverted repeat and a wild-type right inverted repeat. This Cre-recombinase recognition sequence is denoted as Lx-LE and has in forward orientation the sequence of SEQ ID NO: 03 and in reverse orientation the sequence of SEQ ID NO: 04.

In certain embodiments, the Lx spacer sequence is combined with a mutated right inverted repeat and a wild-type left inverted repeat. This Cre-recombinase recognition sequence is denoted as Lx-RE and has in forward orientation the sequence of SEQ ID NO: 05 and in reverse orientation the sequence of SEQ ID NO: 06.

The technical principle underlying the current invention is transcriptional activation of open reading frames or genes by combining DNA-inversion with concomitant operable-linking to a regulatory element, such as, e.g., a promoter.

One independent aspect of the current invention is a double stranded DNA element comprising a (positively oriented) coding strand and a (negatively oriented) template strand,
characterized in that
the coding strand comprises in 5′- to 3′-orientation, i.e. in the following order
   a first promoter,
   a first recombinase recognition sequence comprising a mutation in one of the inverted repeats, i.e. either in the left inverted repeat or in the right inverted repeat, and the other inverted repeat is a not-mutated/wild-type inverted repeat,
   a second promoter that is inverted (in sequence) with respect to the coding strand (direction),
   a first polyadenylation signal and/or transcription termination element, which is inverted (in sequence) with respect to the coding strand (direction),
   a first open reading frame that is inverted (in sequence) with respect to the coding strand (direction) and that is operably linked to the first polyadenylation signal and/or transcription termination element,
   a second recombinase recognition sequence, which comprises a mutation in the respective other inverted repeat as the first recombinase recognition sequence, and which is in inverted/reciprocal orientation with respect to the first recombinase recognition sequence,
   a second open reading frame,
   a second polyadenylation signal and/or transcription termination element, which is operably linked to the second open reading frame.

One independent aspect of the current invention is a double stranded DNA element comprising in 5′- to 3′-direction, i.e. in the following order
a first promoter in 5′- to 3′-orientation/positive orientation,
a first recombinase recognition sequence comprising a mutation in one of the inverted repeats, i.e. either in the left inverted repeat or in the right inverted repeat,
a second promoter in 3′- to 5′-orientation/negative orientation,
a first polyadenylation signal and/or transcription termination element in 3′- to 5′-orientation/negative orientation,
a first open reading frame in 3′- to 5′-orientation/negative orientation and operably operably linked to the first polyadenylation signal and/or transcription termination element,
a second recombinase recognition sequence, which comprises a mutation in the respective other inverted repeat as the first recombinase recognition sequence, and which is in reciprocal/inverted orientation with respect to the first recombinase recognition sequence,
a second open reading frame in 5′- to 3′-orientation/positive orientation,
a second polyadenylation signal and/or transcription termination element, which is operably linked to the second open reading frame.

In certain dependent embodiments, incubation of the double stranded DNA element with a recombinase functional with said first and second recombinase recognition sequence results
in the inversion of the sequence located between the first and the second recombinase recognition sequence, whereafter the first promoter is operably linked to the first open reading frame and the second promoter is operably linked to the second open reading frame, and
in the generation of a (third) recombinase recognition sequence between the first promoter and the first open reading frame or between the second promoter and the second open reading frame following recombinase-mediated inversion of the DNA sequence between said first and second recombinase recognition sequence, which ((third) recombinase recognition sequence) is no-longer functional with said recombinase.

One independent aspect of the current invention is a double stranded adenoviral VA RNA element comprising in 5′- to 3′-direction, i.e. in the following order
a promoter in 5′- to 3′-orientation/positive orientation,
a first recombinase recognition sequence comprising a mutation in one of the inverted repeats, i.e. either in the left inverted repeat or in the right inverted repeat,
an adenoviral VA RNA gene in 3′- to 5′-orientation/negative orientation,
a second recombinase recognition sequence, which comprises a mutation in the respective other inverted repeat as the first recombinase recognition sequence, and which is in reciprocal/inverted orientation with respect to the first recombinase recognition sequence.

In certain dependent embodiments, incubation of the double stranded VA RNA element with a recombinase functional with said first and second recombinase recognition sequence results
in the inversion of the sequence between the first and the second recombinase recognition sequence, whereafter the promoter is operably linked to the VA RNA gene, and
in the generation of a (third) recombinase recognition sequence between the promoter and the VA RNA gene or downstream of the VA RNA gene following recombinase-mediated inversion of the DNA sequence between said first and second recombinase recognition sequence, which ((third) recombinase recognition sequence) is no-longer functional with said recombinase.

One independent aspect of the invention is a (double stranded) DNA (molecule) comprising
a first double stranded DNA element according to the invention,
a second double stranded DNA element according to the invention,
optionally a third double stranded DNA element according to the invention or an adenoviral VA RNA element according to the invention, and
a rep or/and cap open reading frame (element).

In certain dependent embodiments,
1)
   in the first double stranded DNA element the first open reading frame is the E1A open reading frame and the second open reading frame is the E1B open reading frame, or vice versa; and
   in the second double stranded DNA element the first open reading frame is the E2A open reading frame and the second open reading frame is the E4 open reading frame or the E4orf6 (open reading frame), or vice versa,
or
2)
   in the first double stranded DNA element the first open reading frame is the E2A open reading frame and the second open reading frame is the E4 open reading frame or the E4orf6 (open reading frame), or vice versa; and
   in the second double stranded DNA element the first open reading frame is the E1A open reading frame and the second open reading frame is the E1B open reading frame, or vice versa.

One independent aspect of the current invention is a mammalian or insect cell comprising at least one double stranded DNA element or molecule according to the current invention or a (sequence) inverted form thereof.

One independent aspect according to the current invention is a method for producing a recombinant adeno-associated virus (rAAV) vector or particle comprising the following steps:
cultivating/propagating a cell according to the current invention (under conditions suitable for cell division),
activating rAAV vector or particle production by recombinase mediated open reading frame inversion according to the invention (by introducing a recombinase as protein or as mRNA or as DNA in the cell according to the invention, whereby the recombinase is functional with the recombinase recognition sequences in the DNA element or molecule according to the invention),
optionally cultivating the rAAV vector or particle production activated cell obtained in the previous step (under conditions suitable for rAAV vector or particle production),
recovering the rAAV vector or particle from the cells or/and the cultivation medium.

Thus, one independent aspect of the current invention is a (double stranded) DNA (molecule) (for the production of recombinant adeno-associated virus vectors or particles) comprising
a) an E1A open reading frame and an E1B open reading frame; and
b) an E2A open reading frame and an E4 or E4orf6 open reading frame;
characterized in that the first and second open reading frames of a) or b) are comprised/contained in a double stranded DNA element comprising a (positively oriented) coding strand and a (negatively oriented) template strand,
wherein the coding strand comprises in 5′- to 3′-orientation, i.e. in the following order
   a first promoter (in positive orientation),
   a first recombinase recognition sequence comprising a mutation in one of the inverted repeats,
   a second promoter that is inverted (in sequence) with respect to the coding strand (direction) (i.e. is in inverted/negative orientation),
   optionally a first polyadenylation signal and/or transcription termination element that is inverted (in sequence) with respect to the coding strand (direction) (i.e. is in inverted/negative orientation) and that is operably linked to the first open reading frame,
   the first open reading frame (of a) or b)) that is inverted (in sequence) with respect to the coding strand direction (i.e. is in inverted/negative orientation),
   a second recombinase recognition sequence comprising a mutation in the respective other inverted repeat and being in reciprocal/inverted orientation with respect to the first recombinase recognition sequence,
   the second open reading frame of a) if the first open reading frame is of a) or the second open reading frame of b) if the first open reading frame is of b) (in positive orientation),
   optionally a second polyadenylation signal and/or transcription termination element (in positive orientation and operably linked to the second open reading frame).

Thus, one independent aspect of the current invention is a (double stranded) DNA (molecule) (for the production of recombinant adeno-associated virus vectors or particles) comprising
a) an E1A open reading frame and an E1B open reading frame; and
b) an E2A open reading frame and an E4 or E4orf6 open reading frame;
characterized in that the first and the second open reading frames of a) and the first and the second open reading frames b) are each contained in a double stranded DNA element (i.e. the DNA molecule comprises two of said DNA elements) each comprising a (positively oriented) coding strand and a (negatively oriented) template strand,
wherein the coding strand comprises in 5′- to 3′-orientation, i.e. in the following order
   a first promoter (in positive orientation),
   a first recombinase recognition sequence comprising a mutation in one of the inverted repeats,
   a second promoter that is inverted (in sequence) with respect to the coding strand (direction) (i.e. is in inverted/negative orientation),
   optionally a first polyadenylation signal and/or transcription termination element that is inverted (in sequence) with respect to the coding strand (direction) (i.e. is in inverted/negative orientation) and that is operably linked to the first open reading frame,
   the first open reading frame (of a) or b)) that is inverted (in sequence) with respect to the coding strand direction (i.e. is in inverted/negative orientation),
   a second recombinase recognition sequence comprising a mutation in the respective other inverted repeat and being in reciprocal/inverted orientation with respect to the first recombinase recognition sequence,
   the second open reading frame of a) if the first open reading frame is of a) or the second open reading frame of b) if the first open reading frame is of b) (in positive orientation),
   optionally a second polyadenylation signal and/or transcription termination element (in positive orientation and operably linked to the second open reading frame).

Thus, one aspect of the current invention is a (double stranded) DNA (molecule) (for the production of recombinant adeno-associated virus vectors or particles) comprising (at least one) a double stranded DNA element comprising a (positively oriented) coding strand and a (negatively oriented) template strand,
wherein the coding strand comprises in 5′- to 3′-orientation, i.e. in the following order
   a first promoter, in one preferred embodiment the adeno-associated viral promoter P5 or a functional fragment thereof or a variant thereof,
   a first recombinase recognition sequence comprising a mutation in one of the inverted repeats,
   the rep and cap open reading frames including further promoters for the expression of the Rep and Cap proteins, which are inverted (in sequence) with respect to the coding strand (direction) (i.e. in inverted orientation),
   a second recombinase recognition sequence comprising a mutation in the respective other inverted repeat and being in reciprocal/inverted orientation to the first recombinase recognition sequence,
   a polyadenylation signal, in one preferred embodiment the autologous polyadenylation signal of the rep and cap open reading frames.

In certain dependent embodiments, incubation of the (double stranded) DNA (molecule) with a recombinase functional with said first and second recombinase recognition sequence results
in the inversion of the sequence between the first and the second recombinase recognition sequence, whereafter the first promoter is operably linked to the rep and cap open reading frames, and
in the generation of a (third) recombinase recognition sequence between the first promoter and the rep and cap open reading frames or between the rep and cap open reading frames and the polyadenylation signal following recombinase-mediated inversion of the DNA sequence between said first and second recombinase recognition sequence, which the first and the second open reading frames is no-longer functional with said recombinase.

Another independent aspect of the current invention is a (double stranded) DNA (molecule) (for the production of recombinant adeno-associated virus vectors or particles) comprising a double stranded DNA element comprising a (positively oriented) coding strand and a (negatively oriented) template strand,
wherein the coding strand comprises in 5′- to 3′-orientation, i.e. in the following order
   a first promoter, in one preferred embodiment the adeno-associated viral promoter P5 or a functional fragment thereof or a variant thereof,
   a first recombinase recognition sequence comprising a mutation in one of the inverted repeats,
   a second promoter that is inverted with respect to the coding strand (in inverted orientation), in one preferred embodiment the adeno-associated viral promoter P19 or a functional fragment thereof or a variant thereof,
   optionally a first polyadenylation signal and/or transcription termination element that is inverted (in sequence) with respect to the coding strand (direction) (i.e. is in inverted/negative orientation) and that is operably linked to the Rep78 or Rep68 coding sequence,
   a coding sequence, which encodes either exclusively the Rep78 protein or exclusively the Rep68 protein, but not both,
      (i) optionally the internal P40 promoter is inactivated, and/or
      (ii) the start codon of Rep52/40 is mutated into a non-start codon, and/or
      (iii) splice donor and acceptor sites are removed, and which is inverted with respect to the coding strand (in inverted orientation),
   a second recombinase recognition sequence, which comprises a mutation in the respective other inverted repeat as the first recombinase recognition sequence, and which is in reciprocal/inverted orientation with respect to the first recombinase recognition sequence,
   the Rep52/Rep40 and Cap open reading frames including a common polyadenylation signal sequence, i.e. a polyadenylation signal operably linked to said open reading frames.

Another independent aspect of the current invention is a (double stranded) DNA (molecule) (for the production of recombinant adeno-associated virus vectors or particles) comprising a double stranded DNA element comprising a (positively oriented) coding strand and a (negatively oriented) template strand,
wherein the coding strand comprises in 5′- to 3′-orientation, i.e. in the following order
   a first promoter, in one preferred embodiment the adeno-associated viral promoter P5 or a functional fragment thereof or a variant thereof,
   a first recombinase recognition sequence comprising a mutation in one of the inverted repeats,
   a second promoter that is inverted with respect to the coding strand (in inverted orientation), in one preferred embodiment the adeno-associated viral promoter P19 or a functional fragment thereof or a variant thereof,
   optionally a first polyadenylation signal and/or transcription termination element that is inverted (in sequence) with respect to the coding strand (direction) (i.e. is in inverted/negative orientation) and that is operably linked to the Rep78 or Rep68 coding sequence,
   a coding sequence, which encodes either exclusively the Rep78 protein or exclusively the Rep68 protein, but not both,
      (i) optionally the internal P40 promoter is inactivated, and/or
      (ii) the start codon of the Rep52/40 open reading frame is mutated into a non-start codon, and
      (iii) splice donor and acceptor sites are removed, and which is inverted with respect to the coding strand (in inverted orientation),
   a second recombinase recognition sequence, which comprises a mutation in the respective other inverted repeat as the first recombinase recognition sequence, and which is in reciprocal/inverted orientation with respect to the first recombinase recognition sequence,
   the Rep52 open reading frame, optionally with splice donor and acceptor sites removed, or the Rep 40 open reading frame including a polyadenylation signal sequence, i.e. a polyadenylation signal operably linked to said open reading frame,
   optionally a third promoter, a cap open reading frame and a polyadenylation and/or terminator sequence, wherein all are operably linked.

One independent aspect of the current invention is an adenoviral VA RNA gene operably linked to a functional promoter, wherein a precise transcription start site has been added and a Cre-recombinase recognition sequence has been engineered into/within the adenoviral VA RNA gene.

One aspect of the invention is an isolated (mammalian or insect) cell comprising at least one of the DNA element or the DNA (molecule) or the adenoviral VA RNA of the current invention in original or (recombinase) inverted form.

One aspect of the invention is a method of generating/for producing a recombinant adeno-associated virus (rAAV) vector or particle, the method comprising:
providing a mammalian, in suspension growing cell, which comprises
   a transgene expression cassette interspaced between two AAV ITRs;
   open reading frames encoding adenoviral E1A, E1B, E2A, E4 or E4orf6 proteins and adenoviral VA RNA;
   open reading frames encoding adeno-associated Rep/Cap proteins;
   one or more different pairs of non-compatible recombinase recognition sequences;
      wherein individually or in combination one or more from the group consisting of the E1A open reading frame, the E1B open reading frame, the E2A open reading frame, the E4 open reading frame, the E4 open reading frame 6, the Rep78 open reading frame, the Rep68 open reading frame, the Rep52 open reading frame, the Rep40 open reading frame, the Rep/Cap open reading frames and the adenoviral VA RNA gene, is/are each placed without operably linked promoter but including operably linked polyadenylation and/or transcription termination signal between a pair of said non-compatible recombinase recognition sequences, wherein one recombinase recognition sequence comprises a mutation in the left inverted repeat and one recombinase recognition sequence comprises a mutation in the right inverted repeat, with a promoter located upstream of the first recombinase recognition sequence, and the open reading frame being in reverse orientation with respect to the promoter located upstream therefrom;
      wherein the recombinase recognition sequences are organized to allow generation of a recombinase-dependent change that is detectable (e.g. by rAAV vector or particle production), in certain embodiments the one or more recombinase recognition sequences are Cre-recombinase recognition sites (i.e. the recombinase recognition sequences are in reciprocal/inverted orientation with respect to each other and action of the recombinase results in the inversion of the sequence(s) between the recombinase recognition sequences and the concomitant operable linking to the upstream located promoter to the inverted sequence), in certain embodiments the one or more recombinase recognition sequences are Flp-recognition sites (i.e. the recombinase recognition sequences are in reciprocal/inverted orientation with respect to each other and action of the recombinase results in the inversion of the sequence(s) between the recombinase recognition sequences and the concomitant operable linking to the upstream located promoter to the inverted sequence);
inducing expression of the recombinase in said mammalian cell either by transfecting said cell with a recombinase expression plasmid or recombinase mRNA or by activating a conditional recombinase expression within said mammalian cell, whereby the expression of the recombinase results in a recombinase-mediated cassette inversion resulting in rAAV vector or particle production, and wherein the recombinase-mediated cassette inversion is the inversion of the sequence that is flanked by the recombinase recognition sequences;
isolating the rAAV vector or particle from the cell or/and the cultivation medium and thereby producing the rAAV vector or particle.

One aspect of the invention is a method of obtaining site-specific replacement of a DNA of interest in a mammalian cell, comprising:
a) providing a mammalian cell comprising a DNA element according to the current invention;
b) introducing into the cell or activating in the cell a recombinase functional with the recombinase recognition sequences of said DNA element of a);
wherein the recombinase catalyzes the inversion of the sequence between the recombinase recognition sequences and thereby a site-specific replacement of a DNA of interest in a mammalian cell is obtained.

In certain embodiments of all aspects and embodiments, the first recombinase recognition sequence comprises a mutation in the left inverted repeat and the second recombinase recognition sequence comprises a mutation in the right inverted repeat. This arrangement results after recombinase-mediated inversion that the upstream, i.e. 5′-located, recombinase recognition sequence comprises a mutation in both inverted repeats and is thereby non-functional, i.e. cannot be recognized by the respective recombinase. The downstream, i.e. 3′-located, recombinase recognition sequence is wild-type with respect to both inverted repeats and is thereby functional, i.e. can be recognized by the respective recombinase.

In certain embodiments of all aspects and embodiments, the first recombinase recognition sequence comprises a mutation in the right inverted repeat and the second recombinase recognition sequence comprises a mutation in the left inverted repeat. This arrangement results after recombinase-mediated inversion that the downstream, i.e. 3′-located, recombinase recognition sequence comprises a mutation in both inverted repeats and is thereby non-functional, i.e. cannot be recognized by the respective recombinase. The upstream, i.e. 5′-located, recombinase recognition sequence is wild-type with respect to both inverted repeats and is thereby functional, i.e. can be recognized by the respective recombinase.

In certain embodiments of all aspects and embodiments, the first promoter is in positive orientation and/or the second open reading frame is in positive orientation.

### DETAILED DESCRIPTION OF EMBODIMENTS OF THE INVENTION

Herein are reported novel DNA constructs and methods using the same. The novel DNA constructs according to the current invention are useful in the simultaneous transcriptional activation of at least two open reading frames or genes using site-specific, recombinase-mediated cassette inversion (RMCI). The current invention uses a deliberate non-productive arrangement of promoters and open reading frames on coding and template strands of double-stranded DNA molecules, which are converted into their productive, i.e. operably linked, form by the interaction (i.e. inversion) with a site-specific recombinase.

### Definitions

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R.I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987).

The use of recombinant DNA technology enables the generation of derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

Deoxyribonucleic acids comprise a coding and a non-coding strand. The terms “5′” and “3′” when used herein refer to the position on the coding strand.

The term “3′ flanking sequence” denotes a sequence located at the 3′-end (downstream of; below) a nucleotide sequence.

The term “5′ flanking sequence” denotes a sequence located at the 5′-end (upstream of, above) a nucleotide sequence.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The term “AAV helper functions” denotes AAV-derived coding sequences (proteins) which can be expressed to provide AAV gene products and AAV particles that, in turn, function in trans for productive AAV replication and packaging. Thus, AAV helper functions include AAV open reading frames (ORFs), including rep and cap and others such as AAP for certain AAV serotypes. The rep gene expression products have been shown to possess many functions, including, among others: recognition, binding and nicking of the AAV origin of DNA replication; DNA helicase activity; and modulation of transcription from AAV (or other heterologous) promoters. The cap gene expression products (capsids) supply necessary packaging functions. AAV helper functions are used to complement AAV functions in trans that are missing from AAV vector genomes.

The term “about” denotes a range of +/−20% of the thereafter following numerical value. In certain embodiments, the term about denotes a range of +/−10% of the thereafter following numerical value. In certain embodiments, the term about denotes a range of +/−5% of the thereafter following numerical value.

The term “comprising” also encompasses the term “consisting of”.

The term “CAS protein” denotes a CRISPR-associated-protein, which has ribonuclease activity and can bind specific RNA sequences.

The term “CAS9” denotes the endonuclease Cas9. This enzyme binds the RNA sequence GUUUUAGAGCU(A/G)UG(C/U)UGUUUUG (crRNA repeat) (SEQ ID NO: 26) and cuts associated DNA there.

The term “Cre-recombinase” denotes a tyrosine recombinase that catalyzes site-specific recombination using a topoisomerase I-like mechanism between LoxP sites. The molecular weight of the enzyme is about 38 kDa and it consists of 343 amino acid residues. It is a member of the integrase family. An exemplary Cre-recombinase has the amino acid sequence of:

(SEQ ID NO: 07)
MSNLLTVHQN LPALPVDATS DEVRKNLMDM FRDRQAFSEH
TWKMLLSVCR SWAAWCKLNN RKWFPAEPED VRDYLLYLQA
RGLAVKTIQQ HLGQLNMLHR RSGLPRPSDS NAVSLVMRRI
RKENVDAGER AKQALAFERT DFDQVRSLME NSDRCQDIRN
LAFLGIAYNT LLRIAEIARI RVKDISRTDG GRMLIHIGRT
KTLVSTAGVE KALSLGVTKL VERWISVSGV ADDPNNYLFC
RVRKNGVAAP SATSQLSTRA LEGIFEATHR LIYGAKDDSG
QRYLAWSGHS ARVGAARDMA RAGVSIPEIM QAGGWTNVNI
VMNYIRNLDS ETGAMVRLLE DGD;

and one corresponding Cre mRNA has the sequence of:

(SEQ ID NO: 08)
AUGAGCAACC UGCUGACCGU GCACCAGAAC CUGCCCGCCC
UGCCCGUGGA CGCCACCAGC GACGAGGUGA GGAAGAACCU
GAUGGACAUG UUCAGGGACA GGCAGGCCUU CAGCGAGCAC
ACCUGGAAGA UGCUGCUGAG CGUGUGCAGG AGCUGGGCCG
CCUGGUGCAA GCUGAACAAC AGGAAGUGGU UCCCCGCCGA
GCCCGAGGAC GUGAGGGACU ACCUGCUGUA CCUGCAGGCC
AGGGGCCUGG CCGUGAAGAC CAUCCAGCAG CACCUGGGCC
AGCUGAACAU GCUGCACAGG AGGAGCGGCC UGCCCAGGCC
CAGCGACAGC AACGCCGUGA GCCUGGUGAU GAGGAGGAUC
AGGAAGGAGA ACGUGGACGC CGGCGAGAGG GCCAAGCAGG
CCCUGGCCUU CGAGAGGACC GACUUCGACC AGGUGAGGAG
CCUGAUGGAG AACAGCGACA GGUGCCAGGA CAUCAGGAAC
CUGGCCUUCC UGGGCAUCGC CUACAACACC CUGCUGAGGA
UCGCCGAGAU CGCCAGGAUC AGGGUGAAGG ACAUCAGCAG
GACCGACGGC GGCAGGAUGC UGAUCCACAU CGGCAGGACC
AAGACCCUGG UGAGCACCGC CGGCGUGGAG AAGGCCCUGA
GCCUGGGCGU GACCAAGCUG GUGGAGAGGU GGAUCAGCGU
GAGCGGCGUG GCCGACGACC CCAACAACUA CCUGUUCUGC
AGGGUGAGGA AGAACGGCGU GGCCGCCCCC AGCGCCACCA
GCCAGCUGAG CACCAGGGCC CUGGAGGGCA UCUUCGAGGC
CACCCACAGG CUGAUCUACG GCGCCAAGGA CGACAGCGGC
CAGAGGUACC UGGCCUGGAG CGGCCACAGC GCCAGGGUGG
GCGCCGCCAG GGACAUGGCC AGGGCCGGCG UGAGCAUCCC
CGAGAUCAUG CAGGCCGGCG GCUGGACCAA CGUGAACAUC
GUGAUGAACU ACAUCAGGAA CCUGGACAGC GAGACCGGCG
CCAUGGUGAG GCUGCUGGAG GACGGCGAC

or likewise a variant thereof with different codon usage.

The term “CRISPR” is the abbreviation for Clustered Regularly Interspaced Short Palindromic Repeats; grouped short palindromic repeats at regular intervals.

The term “CRISPR/CAS” denotes a CRISPR associated systems. Clustered regulatory interspaced short palindromic repeats are loci that contain multiple short direct repeats, and provide acquired immunity to bacteria and archaea. CRISPR systems rely on crRNA and tracrRNA for sequence-specific silencing of invading foreign DNA. Three types of CRISPR/CAS systems exist: in type II systems, Cas9 serves as an RNA-guided DNA endonuclease that cleaves DNA upon crRNA-tracrRNA target recognition.

The term “crRNA” denotes an RNA consisting of crRNA repeat sequence and crRNA spacer sequence; has a specific secondary structure; crRNA is bound by Cas9, thereby inducing conformational changes in Cas9 whereby target DNA can be bound by the crRNA spacer (complementary to target DNA); by exchanging the crRNA spacer sequence, target DNA can be altered (to target DNA complementary RNA sequence); crRNA repeat consists of 20 nucleotides; the 12 nucleotides adjacent to the PAM motif are crucial for binding specificity.

The term “donor plasmid” denotes a plasmid containing the donor sequence.

The term “donor sequence” denotes a sequence comprising 5′ flanking sequence—target sequence—3′ flanking sequence.

The term “DSB” denotes a double strand break: the product of ZFN, TALEN, and CRISPR/Cas9 action, double-strand breaks are a form of DNA damage that occurs when both DNA strands are cleaved.

The terms “empty capsid” and “empty particle”, refer to an AAV particle that has an AAV protein shell but that lacks in whole or part a nucleic acid that encodes a protein or is transcribed into a transcript of interest flanked by AAV ITRs, i.e. a vector. Accordingly, the empty capsid does not function to transfer a nucleic acid that encodes a protein or is transcribed into a transcript of interest into the host cell.

The term “endogenous” denotes that something is naturally occurring within a cell; naturally produced by a cell; likewise an “endogenous gene locus/cell-endogenous gene locus” is a naturally occurring locus in a cell.

As used herein, the term “exogenous” indicates that a nucleotide sequence does not originate from a specific cell and is introduced into said cell by DNA delivery methods, e.g., by transfection, electroporation, or transduction by viral vectors. Thus, an exogenous nucleotide sequence is an artificial sequence wherein the artificiality can originate, e.g., from the combination of subsequences of different origin (e.g. a combination of a recombinase recognition sequence with an SV40 promoter and a coding sequence of green fluorescent protein is an artificial nucleic acid) or from the deletion of parts of a sequence (e.g. a sequence coding only the extracellular domain of a membrane-bound receptor or a cDNA) or the mutation of nucleobases. The term “endogenous” refers to a nucleotide sequence originating from a cell. An “exogenous” nucleotide sequence can have an “endogenous” counterpart that is identical in base compositions, but where the sequence is becoming an “exogenous” sequence by its introduction into the cell, e.g., via recombinant DNA technology.

As used herein, the term “flanking” denotes that a first nucleotide sequence is located at either a 5′- or 3′-end, or both ends of a second nucleotide sequence. The flanking nucleotide sequence can be adjacent to or at a defined distance from the second nucleotide sequence. There is no specific limit of the length of a flanking nucleotide sequence beside practical requirements. For example, a flanking sequence can be a few base pairs or a few thousand base pairs. The term “flanking nucleotide sequence” denotes a sequence segment of a nucleic acid that precedes or follows the sequence to be inserted (=target sequence).

The term “gene locus” denotes the location of a gene on a chromosome, i.e. the position of a gene in the genome, i.e. the gene location.

The term “HR” denotes homologous recombination: homology-directed repair is a template-dependent pathway for DSB repair. By supplying a homology-containing donor template along with a site-specific nuclease, HDR faithfully inserts the donor molecule at the targeted locus. This approach enables the insertion of single or multiple transgenes, as well as single nucleotide substitutions.

An “isolated” composition is one, which has been separated from one or more component(s) of its natural environment. In some embodiments, a composition is purified to greater than 95% or 99% purity as determined by, for example, electrophoretic (e.g., SDS-PAGE, isoelectric focusing (IEF), capillary electrophoresis, CE-SDS) or chromatographic (e.g., size exclusion chromatography or ion exchange or reverse phase HPLC). For review of methods for assessment of e.g. antibody purity, see, e.g., Flatman, S. et al., J. Chrom. B 848 (2007) 79-87.

An “isolated” nucleic acid refers to a nucleic acid molecule that has been separated from one or more component(s) of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated” polypeptide or antibody refers to a polypeptide molecule or antibody molecule that has been separated from one or more component(s) of its natural environment.

The term “integration site” denotes a nucleic acid sequence within a cell's genome into which an exogenous nucleotide sequence is/has been inserted. In certain embodiments, an integration site is between two adjacent nucleotides in the cell's genome. In certain embodiments, an integration site includes a stretch of nucleotides. In certain embodiments, the integration site is located within a specific locus of the genome of a mammalian cell. In certain embodiments, the integration site is within an endogenous gene of a mammalian cell.

The term “LoxP site” denotes a nucleotide sequence of 34 bp in length consisting of two palindromic 13 bp sequences (inverted repeats) at the termini (ATAACTTCGTATA (SEQ ID NO: 14) and TATACGAAGTTAT (SEQ ID NO: 15), respectively) and a central 8 bp core (not symmetric) spacer sequence. The spacer sequences determine the orientation of the LoxP site. Depending on the relative orientation and location of two LoxP sites with respect to each other, the intervening DNA is either excised (LoxP sites oriented in the same direction) or inverted (LoxP sites orientated in opposite directions). The term “floxed” denotes a DNA sequence located between two LoxP sites. If there are two floxed sequences, i.e. a target floxed sequence in the genome and a floxed sequence in a donor nucleic acid, both sequences can be exchanged with each other. This is called “recombinase-mediated cassette exchange”.

Exemplary LoxP sites are shown in the following Table:

| name | core sequence | SEQ ID NO: |
|---|---|---|
| LoxP | ATGTATGC | 16 |
| L3 | AAGTCTCC | 17 |
| L2 (inverted) | GCATACAT | 18 |
| LoxFas | TACCTTTC | 19 |
| Lox511 | ATGTATAC | 20 |
| Lox5171 | ATGTGTAC | 21 |
| Lox2272 | AAGTATCC | 22 |
| Loxm2 | AGAAACCA | 23 |
| Loxm3 | TAATACCA | 24 |
| Loxm7 | AGATAGAA | 25 |

The term “mammalian cell comprising an exogenous nucleotide sequence” encompasses cells into which one or more exogenous nucleic acid(s) have been introduced, including the progeny of such cells. These can be the starting point for further genetic modification. Thus, the term “a mammalian cell comprising an exogenous nucleotide sequence” encompasses a cell comprising an exogenous nucleotide sequence integrated at a single site within a locus of the genome of said mammalian cell, wherein the exogenous nucleotide sequence comprises at least a first and a second recombination recognition site (these recombination recognition sites are different) flanking at least one first selection marker. In certain embodiments, the mammalian cell comprising an exogenous nucleotide sequence is a cell comprising an exogenous nucleotide sequence integrated at a single site within a locus of the genome of said cell, wherein the exogenous nucleotide sequence comprises a first and a second recombination recognition sequence flanking at least one first selection marker, and a third recombination recognition sequence located between the first and the second recombination recognition sequence, and all the recombination recognition sequences are different.

A “mammalian cell comprising an exogenous nucleotide sequence” and a “recombinant cell” are both “transfected cells”. This term includes the primary transfected cell as well as progeny derived therefrom without regard to the number of passages. Progeny may, e.g., not be completely identical in nucleic acid content to a parent cell, but may contain mutations. Mutant progeny that has the same function or biological activity as in the originally transfected cell are encompassed.

The term “NHEJ” denotes non-homologous end joining. This is a DSB repair pathway that ligates or joins two broken ends together. NHEJ does not use a homologous template for repair and thus typically leads to the introduction of small insertions and deletions at the site of the break, often inducing frame-shifts that knockout gene function.

The term “non-compatible” as used herein denotes a recombinase recognition site, such as, e.g., a first LoxP site, that does not recombine with another recombinase recognition site, such as, e.g., a second LoxP site with which it does not share spacer region homology. In certain embodiments, the non-compatible LoxP site recombines with another LoxP site with which it does not share spacer region homology to less than 1%, in one preferred embodiment to 0.5% or less. That means that two non-compatible LoxP sites linked in cis are stable in the presence of Cre-recombinase, i.e. at most 1% of the sites exchange, in a preferred embodiment 0.5% or less of the sites exchange.

The term “nuclear localization sequence” as used herein denotes an amino acid sequence comprising multiple copies of the positively charged amino acid residue arginine or/and lysine. A polypeptide comprising said sequence is identified by the cell for import into the cell nucleus. Exemplary nuclear localization sequences are PKKKRKV (SEQ ID NO: 09; SV40 large T-antigen), KR[PAATKKAGQA]KKKK (SEQ ID NO: 10, SV40 nucleoplasmin), MSRRRKANPTKLSENAKKLAKEVEN (SEQ ID NO: 11; Caenorhabditis elegans EGL-13), PAAKRVKLD (SEQ ID NO: 12, human c-myc), KLKIKRPVK (SEQ ID NO: 13, E. coli terminus utilization substance protein). Other nuclear localization sequences can be identified easily by a person skilled in the art.

The “nucleic acids encoding AAV packaging proteins” refer generally to one or more nucleic acid molecule(s) that includes nucleotide sequences providing AAV functions deleted from an AAV vector, which is(are) to be used to produce a transduction competent recombinant AAV particle. The nucleic acids encoding AAV packaging proteins are commonly used to provide expression of AAV rep and/or cap genes to complement missing AAV functions that are necessary for AAV replication; however, the nucleic acid constructs lack AAV ITRs and can neither replicate nor package themselves. Nucleic acids encoding AAV packaging proteins can be in the form of a plasmid, phage, transposon, cosmid, virus, or particle. A number of nucleic acid constructs have been described, such as the commonly used plasmids pAAV/Ad and pIM29+45, which encode both rep and cap gene expression products. See, e.g., Samulski et al. (1989) J. Virol. 63:3822-3828; and McCarty et al. (1991) J. Virol. 65:2936-2945. A number of plasmids have been described which encode rep and/or cap gene expression products (e.g., U.S. Pat. Nos. 5,139,941 and 6,376,237). Any one of these nucleic acids encoding AAV packaging proteins can comprise the DNA element or nucleic acid according to the invention.

The term “nucleic acids encoding helper proteins” refers generally to one or more nucleic acid molecule(s) that include nucleotide sequences encoding proteins and/or RNA molecules that provide adenoviral helper function(s). A plasmid with nucleic acid(s) encoding helper protein(s) can be transfected into a suitable cell, wherein the plasmid is then capable of supporting AAV particle production in said cell. Any one of these nucleic acids encoding helper proteins can comprise the DNA element or nucleic acid according to the invention. Expressly excluded from the term are infectious viral particles, as they exist in nature, such as adenovirus, herpesvirus or vaccinia virus particles.

As used herein, the term “operably linked” refers to a juxtaposition of two or more components, wherein the components are in a relationship permitting them to function in their intended manner. For example, a promoter and/or an enhancer is operably linked to a coding sequence/open reading frame/gene if the promoter and/or enhancer acts to modulate the transcription of the coding sequence/open reading frame/gene. In certain embodiments, DNA sequences that are “operably linked” are contiguous. In certain embodiments, e.g., when it is necessary to join two protein encoding regions, such as a secretory leader and a polypeptide, the sequences are contiguous and in the same reading frame. In certain embodiments, an operably linked promoter is located upstream of the coding sequence/open reading frame/gene and can be adjacent to it. In certain embodiments, e.g., with respect to enhancer sequences modulating the expression of a coding sequence/open reading frame/gene, the two components can be operably linked although not adjacent. An enhancer is operably linked to a coding sequence/open reading frame/gene if the enhancer increases transcription of the coding sequence/open reading frame/gene. Operably linked enhancers can be located upstream, within, or downstream of coding sequences/open reading frames/genes and can be located at a considerable distance from the promoter of the coding sequence/open reading frame/gene.

The term “packaging proteins” refers to non-AAV derived viral and/or cellular functions upon which AAV is dependent for its replication. Thus, the term captures proteins and RNAs that are required in AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-I) and vaccinia virus.

As used herein, “AAV packaging proteins” refer to AAV-derived sequences, which function in trans for productive AAV replication. Thus, AAV packaging proteins are encoded by the major AAV open reading frames (ORFs), rep and cap. The rep proteins have been shown to possess many functions, including, among others: recognition, binding and nicking of the AAV origin of DNA replication; DNA helicase activity; and modulation of transcription from AAV (or other heterologous) promoters. The cap (capsid) proteins supply necessary packaging functions. AAV packaging proteins are used herein to complement AAV functions in trans that are missing from AAV vectors.

The term “PAM motif” denotes a protospacer adjacent motif; motif adjacent to the protospacer; Sequence NGG; in the target DNA; cutting of the target DNA takes place three nucleotides before the PAM.

A “plasmid” is a form of nucleic acid or polynucleotide that typically has additional elements for expression (e.g., transcription, replication, etc.) or propagation (replication) of the plasmid. A plasmid as used herein also can be used to reference such nucleic acid or polynucleotide sequences. Accordingly, in all aspects the inventive compositions and methods are applicable to nucleic acids, polynucleotides, as well as plasmids, e.g., for producing cells that produce viral (e.g., AAV) vectors, to produce viral (e.g., AAV) particles, to produce cell culture medium that comprises viral (e.g., AAV) particles, etc.

The term “proteinaceous compound” as used herein denotes a heteromultimeric molecule comprising at least one polypeptide, which has been produced in functional form in a mammalian cell. Exemplary proteinaceous compounds are adeno-associated virus particles (AAV particles) comprising a capsid formed of capsid polypeptides and a single stranded DNA molecule, which is a non-polypeptide component.

The term “recombinant cell” as used herein denotes a cell after final genetic modification, such as, e.g., a cell expressing a polypeptide of interest or producing a rAAV particle of interest and that can be used for the production of said polypeptide of interest or rAAV particle of interest at any scale. For example, “a mammalian cell comprising an exogenous nucleotide sequence” that has been subjected to recombinase mediated cassette exchange (RMCE) whereby the coding sequences for a polypeptide of interest have been introduced into the genome of the host cell is a “recombinant cell”. Although the cell is still capable of performing further RMCE reactions, it is not intended to do so.

A “recombinant AAV vector” is derived from the wild-type genome of a virus, such as AAV by using molecular biological methods to remove the wild type genome from the virus (e.g., AAV), and replacing it with a non-native nucleic acid, such as a nucleic acid transcribed into a transcript or that encodes a protein. Typically, for AAV one or both inverted terminal repeat (ITR) sequences of the wild-type AAV genome are retained in the recombinant AAV vector. A “recombinant” AAV vector is distinguished from a wild-type viral AAV genome, since all or a part of the viral genome has been replaced with a non-native (i.e., heterologous) sequence with respect to the viral genomic nucleic acid. Incorporation of a non-native sequence therefore defines the viral vector (e.g., AAV) as a “recombinant” vector, which in the case of AAV can be referred to as a “rAAV vector.”

A recombinant vector (e.g., AAV) sequence can be packaged—referred to herein as a “particle”—for subsequent infection (transduction) of a cell, ex vivo, in vitro or in vivo. Where a recombinant vector sequence is encapsulated or packaged into an AAV particle, the particle can also be referred to as a “rAAV”. Such particles include proteins that encapsulate or package the vector genome. Particular examples include viral envelope proteins, and in the case of AAV, capsid proteins, such as AAV VP1, VP2 and VP3.

A “recombination recognition site” (RRS) is a nucleotide sequence recognized by a recombinase and is necessary and sufficient for recombinase-mediated recombination events. A RRS can be used to define the position where a recombination event will occur in a nucleotide sequence.

As used herein, the term “selection marker” denotes a gene that allows cells carrying the gene to be specifically selected for or against, in the presence of a corresponding selection agent. For example, but not by way of limitation, a selection marker can allow the host cell transformed with the selection marker gene to be positively selected for in the presence of the respective selection agent (selective cultivation conditions); a non-transformed host cell would not be capable of growing or surviving under the selective cultivation conditions. Selection markers can be positive, negative or bi-functional. Positive selection markers can allow selection for cells carrying the marker, whereas negative selection markers can allow cells carrying the marker to be selectively eliminated. A selection marker can confer resistance to a drug or compensate for a metabolic or catabolic defect in the host cell. In prokaryotic cells, amongst others, genes conferring resistance against ampicillin, tetracycline, kanamycin or chloramphenicol can be used. Resistance genes useful as selection markers in eukaryotic cells include, but are not limited to, genes for aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid. Further marker genes are described in WO 92/08796 and WO 94/28143.

Beyond facilitating a selection in the presence of a corresponding selection agent, a selection marker can alternatively be a molecule normally not present in the cell, e.g., green fluorescent protein (GFP), enhanced GFP (eGFP), synthetic GFP, yellow fluorescent protein (YFP), enhanced YFP (eYFP), cyan fluorescent protein (CFP), mPlum, mCherry, tdTomato, mStrawberry, J-red, DsRed-monomer, mOrange, mKO, mCitrine, Venus, YPet, Emerald, CyPet, mCFPm, Cerulean, and T-Sapphire. Cells expressing such a molecule can be distinguished from cells not harboring this gene, e.g., by the detection or absence, respectively, of the fluorescence emitted by the encoded polypeptide.

As used herein, the term “serotype” is a distinction based on AAV capsids being serologically distinct. Serologic distinctiveness is determined on the basis of the lack of cross-reactivity between antibodies to one AAV as compared to another AAV.

Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

Under the traditional definition, a serotype means that the virus of interest has been tested against serum specific for all existing and characterized serotypes for neutralizing activity and no antibodies have been found that neutralize the virus of interest. As more naturally occurring virus isolates are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

The term “sgRNA” denotes a single guide RNA; single RNA strand containing the crRNA and tracerRNA.

The term “TALENs” denotes a transcription activator-like effector nuclease. These are fusions of the Fokl cleavage domain and DNA-binding domains derived from TALE proteins. TALEs contain multiple 33-35-amino-acid repeat domains that each recognizes a single base pair. Like ZFNs, TALENs induce targeted DSBs that activate DNA damage response pathways and enable custom alterations.

The term “tracrRNA” denotes a trans-acting CRISPR RNA; non-coding RNA; partially complementary to the crRNA; forms an RNA double helix; promotes crRNA processing; activation by RNase III; binds target DNA; endonuclease function cuts near the binding site; required for activating RNA-guided cleavage by CAS9.

The terms “transduce” and “transfect” refer to introduction of a molecule such as a nucleic acid (viral vector, plasmid) into a cell. A cell has been “transduced” or “transfected” when exogenous nucleic acid has been introduced inside the cell membrane. Accordingly, a “transduced cell” is a cell into which a “nucleic acid” or “polynucleotide” has been introduced, or a progeny thereof in which an exogenous nucleic acid has been introduced. In particular embodiments, a “transduced” cell (e.g., in a mammal, such as a cell or tissue or organ cell) has a genetic change following incorporation of an exogenous molecule, for example, a nucleic acid (e.g., a transgene). A “transduced” cell(s) can be propagated and the introduced nucleic acid transcribed and/or protein expressed.

In a “transduced” or “transfected” cell, the nucleic acid (viral vector, plasmid) may or may not be integrated into genomic nucleic acid. If an introduced nucleic acid becomes integrated into the nucleic acid (genomic DNA) of the recipient cell or organism, it can be stably maintained in that cell or organism and further passed on to or inherited by progeny cells or organisms of the recipient cell or organism. Finally, the introduced nucleic acid may exist in the recipient cell or host organism extrachromosomally, or only transiently. A number of techniques are known, see, e.g., Graham et al. (1973) Virology, 52:456; Sambrook et al. (1989) Molecular Cloning, a laboratory manual, Cold Spring Harbor Laboratories, New York; Davis et al. (1986) Basic Methods in Molecular Biology, Elsevier; and Chu et al. (1981) Gene 13:197. Such techniques can be used to introduce one or more exogenous DNA moieties into suitable host cells.

The term “transgene” is used herein to conveniently refer to a nucleic acid that is intended or has been introduced into a cell or organism. Transgenes include any nucleic acid, such as a gene that is transcribed into a transcript or that encodes a polypeptide or protein.

A “vector” refers to the portion of the recombinant plasmid sequence ultimately packaged or encapsulated, either directly or in form of a single strand or RNA, to form a viral (e.g., AAV) particle. In cases recombinant plasmids are used to construct or manufacture recombinant viral particles, the viral particle does not include the portion of the “plasmid” that does not correspond to the vector sequence of the recombinant plasmid. This non-vector portion of the recombinant plasmid is referred to as the “plasmid backbone”, which is important for cloning and amplification of the plasmid, a process that is needed for propagation and recombinant virus production, but is not itself packaged or encapsulated into virus (e.g., AAV) particles. Thus, a “vector” refers to the nucleic acid that is packaged or encapsulated by a virus particle (e.g., AAV).

The term “ZFN” denotes a zinc-finger nuclease. These are fusions of the nonspecific DNA cleavage domain from the Fokl restriction endonuclease with zinc-finger proteins. ZFN dimers induce targeted DNA DSBs that stimulate DNA damage response pathways. The binding specificity of the designed zinc-finger domain directs the ZFN to a specific genomic site.

The term “ZFNickases” denotes a zinc-finger nickases. These ZFNs contain inactivating mutations in one of the two Fokl cleavage domains. ZFNickases make only single-strand DNA breaks and induce HDR without activating the mutagenic NHEJ pathway.

Gene Editing Methods

Approaches enabling the manipulation of virtually any gene in a diverse range of cell types and organisms have evolved during the past decades. Such a technology is commonly referred to as “genome editing”.

Nucleases

One way of performing genome editing is based on the use of engineered nucleases. These are composed of sequence-specific DNA-binding domains fused to a non-specific DNA cleavage module. Such chimeric nucleases enable efficient and precise genetic modifications by inducing targeted DNA double-strand breaks (DSBs) that stimulate the cellular DNA repair mechanisms, including error-prone non-homologous end joining (NHEJ) and homology-directed repair (HR). The versatility of these methods arises from the ability to customize the DNA-binding domain to recognize virtually any sequence.

Thus, the ability to execute genetic alterations depends largely on the DNA-binding specificity and affinity of the designed proteins (Gaj, T., et al., Trends Biotechnol. 31 (2013) 397-405).

Targeted nucleic acid replacement introduces by homologous recombination between a chromosomal nucleic acid sequence and an exogenous donor nucleic acid sequence site-specific nucleic acid exchanges. Making directed genetic changes is often called “gene targeting” (see, e.g., Carroll, D., Genetics, 188 (2011) 773-782).

Zinc-finger nucleases (ZFNs) and transcription activator-like effector nucleases (TALENs) as well as CRISPR/CAS represent tools for targeted nucleic acid replacement. Clustered regulatory interspaced short palindromic repeat (CRISPR)/CAS-based RNA-guided DNA endonucleases rely on crRNA and tracrRNA for sequence-specific modification of DNA. Three types of CRISPR/CAS systems exist. In type II systems, for example, CAS9 serves as an RNA-guided DNA endonuclease that cleaves DNA upon crRNA-tracrRNA target recognition.

By co-delivering a site-specific nuclease with a donor plasmid bearing locus-specific homology arms, single or multiple transgenes, i.e. exogenous nucleic acids comprising expression cassettes, can be efficiently integrated into a chromosomal target locus. Large transgenes (up to 14 kbps) have been introduced into various endogenous loci via NHEJ-mediated ligation by synchronizing nuclease-mediated cleavage of donor DNA with the chromosomal target (Gaj, T., et al., Trends Biotechnol. 31 (2013) 397-405).

If a double-stranded DNA “donor template” is supplied, HR of a nuclease-induced DSB can be used to introduce precise nucleic acid substitutions or insertions of up to 7.6 kbps at or near the site of the break. Oligonucleotides can be used with ZFNs to introduce precise alterations, small insertions, and large deletions. ZFNs have been used to introduce NHEJ- or HR-mediated gene alterations (Joung, J. K. and Sander, J. D., Nat. Rev. Mol. Cell Biol. 14 (2013) 49-55).

Typically, nuclease-encoded genes are delivered into cells by plasmid DNA, viral vectors, or in vitro transcribed mRNA. Transfection of plasmid DNA or mRNA can be done by electroporation or cationic lipid-based reagents. Integrase-deficient lentiviral vectors (IDLVs) can be used for delivering nucleases into transfection-resistant cell types. AAV can also be used for nuclease delivery.

Zink-Finger-Nucleases (ZFN)

Zink-finger-nucleases, which combine the non-specific cleavage domain (N) of Fokl endo-nuclease with zinc finger proteins (ZFPs), offer a general way to introduce a site-specific double-strand break (DSB) in the genome.

The modular structure of zinc finger (ZF) motifs and modular recognition by ZF domains make them the versatile DNA recognition motifs for designing artificial DNA-binding proteins. Each ZF motif consists of approx. 30 amino acids and folds into BBa structure, which is stabilized by chelation of a zinc ion by the conserved Cys2His2 residues. The ZF motifs bind DNA by inserting the a-helix into the major groove of the DNA double helix. Each finger primarily binds to a triplet within the DNA substrate. Key amino acid residues at positions −1, +1, +2, +3, +4, +5 and +6 relative to the start of the a-helix of each ZF motifs contribute to most of the sequence-specific interactions with the DNA site. These amino acids can be changed while maintaining the remaining amino acids as a consensus backbone to generate ZF motifs with different triplet sequence-specificities. Binding to longer DNA sequences is achieved by linking several of these ZF motifs in tandem to form ZFPs. The designed ZFPs provide a powerful technology since other functionalities like non-specific Fokl cleavage domain (N), transcription activator domains (A), transcription repressor domains (R) and methylases (M) can be fused to a ZFPs to form ZFNs respectively, zinc finger transcription activators (ZFA), zinc finger transcription repressors (ZFR) and zinc finger methylases (ZFM).

Fokl restriction enzyme, a bacterial type IIS restriction endonuclease, recognizes the non-palindromic penta deoxy-ribonucleotide, 5′-GGATG-3′:5′-CATCC-3′ (SEQ ID NO: 27), in duplex DNA and cleaves 9/13 nt downstream of the recognition site. Durai et al. suggested that it is possible to swap the Fokl recognition domain with other naturally occurring DNA-binding proteins that recognize longer DNA sequences or other designed DNA-binding motifs to create chimeric nucleases (Durai, S., et al., Nucl. Acids Res. 33 (2005) 5978-5990).

The Fokl nuclease functions as a dimer and therefore two zinc finger arrays must be designed for each target site. The use of obligate heterodimeric Fokl domains reduce the formation of unwanted homodimeric species and therefore have improved specificities (Joung, J. K. and Sander, J. D., Nat. Rev. Mol. Cell Biol. 14 (2013) 49-55). Thus, a ZFN target sites consist of two zinc-finger binding sites separated by a 5 to 7 bp spacer sequence recognized by the Fokl cleavage domain (Gaj, T., et al., Trends Biotechnol. 31 (2013) 397-405).

Transcription Activator-Like Effector Nucleases (TALENs)

Fusions of transcription activator-like (TAL) effectors of plant pathogenic Xanthomonas spp. to the Fokl nuclease resulted in TALENs. These bind and cleave DNA in pairs. Binding specificity is determined by customizable arrays of polymorphic amino acid repeats in the TAL effectors.

TAL effectors (TALE) enter the nucleus, bind to effector-specific sequences in host gene promoters and activate transcription. Their targeting specificity is determined by a central domain of tandem, 33-35 amino acid repeats, followed by a single truncated repeat of 20 amino acids. Naturally occurring recognition sites are uniformly preceded by a T that is required for TAL effector activity (Cermak, T., et al., Nucl. Acids Res. 39 (2011) e82).

TALE specificity is determined by two hypervariable amino acids that are known as the repeat-variable di-residues (RVDs). Like zinc fingers, modular TALE repeats are linked together to recognize contiguous DNA sequences (Gaj, T., et al., Trends Biotechnol. 31 (2013) 397-405).

TAL effectors can be fused to the catalytic domain of the Fokl nuclease to create targeted DNA double-strand breaks (DSBs) in vivo for genome editing. Since Fokl cleaves as a dimer, these TAL effector nucleases (TALENs) function in pairs, binding opposing targets across a spacer over which the Fokl domains come together to create the break. DSBs are repaired in nearly all cells by one of two highly conserved processes, non-homologous end joining (NHEJ) and homologous recombination (HR), which can be used for gene insertion or replacement.

Assembly of a TALEN or TAL effector construct involves two steps: (i) assembly of repeat modules into intermediary arrays of 1-10 repeats and (ii) joining of the intermediary arrays into a backbone to make the final construct (Cermak, T., et al., Nucl. Acids Res. 39 (2011) e82).

TALEN target sites consist of two TALE binding sites separated by a spacer sequence of varying length (12-20 bp) (Gaj, T., et al., Trends Biotechnol. 31 (2013) 397-405).

For typical heterodimeric target sites (i.e. such as would typically occur in a native DNA sequence), paired TALEN constructs are transformed together into the target cell.

One of the pairs of TALENs directed to the target nucleic acid is subcloned into a mammalian expression plasmid using suitable restriction endonucleases. The resulting plasmids are introduced into target cells by transfection using LipofectAmine 2000 (Invitrogen) following the manufacturer's protocol. Cells are collected 72 hours after transfection (Cermak, T., et al., Nucl. Acids Res. 39 (2011) e82).

Clustered Regularly Interspaced Short Palindromic Repeats (CRISPR)/CRISPR-Associated Protein 9 (CRISPR/CAS9)

The naturally occurring CRISPR/CAS Type II system has been developed into powerful genetic editing tool for eukaryotic cells. Particularly the demonstration that crRNA and tracrRNA can be combined into a single guide RNA (sgRNA) paved the way for this development. Cas9 produces a single double-stranded break in the DNA. The method makes use of DNA repair pathways in eukaryotic cells to provide two ways to make genetic alterations. The first relies on non-homologous end joining (NHEJ) that joins the cut ends. In the second, homology directed repair (HDR) is used to repair the damaged allele using another piece of DNA with homology to the target. By providing a DNA element that can be inserted by recombination, any type of insertion, deletion or change in sequence can be achieved (Rath, D., et al., Biochim. 117 (2015) 119-128).

In the type II CRISPR/CAS system, short segments of foreign DNA, termed ‘spacers’ are integrated within the CRISPR genomic loci and transcribed and processed into short CRISPR RNA (crRNA). These crRNAs anneal to trans-activating crRNAs (tracrRNAs) and direct sequence-specific cleavage and silencing of pathogenic DNA by CAS proteins. It has been shown that target recognition by the Cas9 protein requires a ‘seed’ sequence within the crRNA and a conserved dinucleotide-containing protospacer adjacent motif (PAM) sequence upstream of the crRNA-binding region. The CRISPR/CAS system has been shown to be directly portable to human cells by co-delivery of plasmids expressing the Cas9 endo-nuclease and the necessary crRNA components (Gaj, T., et al., Trends Biotechnol. 31 (2013) 397-405).

Recombinant Cell Line Generation

Generally, for efficient as well as large-scale production of a proteinaceous compound of interest, such as e.g. a rAAV particle or a therapeutic polypeptide, a cell stably expressing and, if possible, also secreting said proteinaceous compound is required. Such a cell is termed “recombinant cell” or “recombinant production cell”. The process for generating such a recombinant cell is termed “cell line development” (CLD).

In a first step, a suitable host cell is transfected with the required nucleic acid sequences encoding said proteinaceous compound of interest. Transfection of additional helper polypeptides may be necessary. In a second step, a cell stably expressing the proteinaceous compound of interest is selected. This can be done, e.g., based on the co-expression of a selection marker, which had been co-transfected with the nucleic acid sequences encoding the proteinaceous compound of interest, or be the expression of the proteinaceous compound itself.

For expression of a coding sequence, i.e. of an open reading frame, additional regulatory elements, such as a promoter and polyadenylation signal (sequence), are necessary. Thus, an open reading frame is operably linked to said additional regulatory elements for transcription. This can be achieved by integrating it into a so-called expression cassette. The minimal regulatory elements required for an expression cassette to be functional in a mammalian cell are a promoter functional in said mammalian cell, which is located upstream, i.e. 5′, to the open reading frame, and a polyadenylation signal (sequence) functional in said mammalian cell, which is located downstream, i.e. 3′, to the open reading frame. Additionally a terminator sequence may be present 3′ to the polyadenylation signal (sequence). For expression, the promoter, the open reading frame/coding region and the polyadenylation signal sequence have to be arranged in an operably linked form.

Likewise, a nucleic acid that is transcribed into a non-protein coding RNA is called “RNA gene”. Also for expression of an RNA gene, additional regulatory elements, such as a promoter and a transcription termination signal or polyadenylation signal (sequence), are necessary. The nature and localization of such elements depends on the RNA polymerase that is intended to drive the expression of the RNA gene. Thus, an RNA gene is normally also integrated into an expression cassette.

In case the proteinaceous compound of interest is a heteromultimeric polypeptide, which is composed of different (monomeric) polypeptides, not only a single expression cassette is required but one for each of the different polypeptides, i.e. open reading frames/coding sequences, as well as RNA genes, if present. These expression cassettes differ at least in the contained open reading frame/coding sequences but can also differ in the promoter and/or polyadenylation signal sequence.

For example, in case the proteinaceous compound of interest is a full length antibody, which is a heteromultimeric polypeptide comprising two copies of a light chain as well as two copies of a heavy chain, two different expression cassettes are required, one for the light chain and one for the heavy chain. If, for example, the full-length antibody is a bispecific antibody, i.e. the antibody comprises two different binding sites specifically binding to two different antigens, each of the light chains as well as each of the heavy chains are also different from each other. Thus, a bispecific full-length antibody is composed of four different polypeptides and, therefore, four expression cassettes containing the four different open reading frames encoding the four different polypeptides are required.

In case the proteinaceous compound of interest is an AAV particle, which is composed of different (monomeric) polypeptides and a single stranded DNA molecule and which in addition requires other co-factors for production and encapsulation, a multitude of expression cassettes differing in the contained open reading frames/coding sequences are required. In this case, at least an expression cassette for each of the transgene, the different polypeptides forming the capsid of the AAV vector, for the required helper functions as well as the VA RNA are required. Thus, individual expression cassettes for each of the helper E1A, E1B, E2A, E4orf6, the VA RNA, the rep and cap genes are required.

As outlined in the previous paragraphs, the more complex the proteinaceous compound of interest or the higher the number of additional required helper polypeptides and/or RNAs, respectively, the higher is the number of required, different expression cassettes. Inherently with the number of expression cassettes, also the size of the nucleic acid to be integrated into the genome of the host cell increases. However, there is a practical upper limit to the size of a nucleic acid that can be transferred, which is in the range of about 15 kbps (kilo-base-pairs). Above this limit handling and processing efficiency profoundly drops. This issue can be addressed by using two or more separate nucleic acids. Thereby the different expression cassettes are allocated to different nucleic acids, whereby each nucleic acid comprises only some of the expression cassettes.

For cell line development random integration (RI) of the nucleic acid(s) carrying the expression cassettes for the proteinaceous compound of interest can be used. In general, by using RI the nucleic acids or fragments thereof integrate into the host cell's genome at random.

Alternatively, to RI, targeted integration (TI) can be used for CLD. In TI CLD, one or more nucleic acid(s) comprising the different expression cassettes is/are introduced at a predetermined locus in the host cell's genome.

In TI either homologous recombination or a recombinase mediated cassette exchange reaction (RMCE) can be employed for the integration of the nucleic acid(a) comprising the respective expression cassettes into the specific locus in the genome of the TI host cell.

In certain embodiments, a method for targeted integration of a single deoxyribonucleic acid into the genome of a (host) mammalian cell (i.e. a method for producing a recombinant mammalian cell), which thereafter comprises a nucleic acid encoding a proteinaceous compound and which thereafter produces said proteinaceous compound, comprising the following steps is provided:
a) providing a mammalian cell comprising an exogenous nucleotide sequence integrated at a defined (optionally single) site within a locus of the genome of the mammalian cell, wherein the exogenous nucleotide sequence comprises a first and a second recombination sequence flanking at least one first selection marker, whereby all recombination sequences are different or/and non-compatible (i.e. these do not result in cross-exchange reactions);
b) introducing into the mammalian cell provided in a) a deoxyribonucleic acid comprising two different recombination sequences and one to eight expression cassettes, wherein
   said deoxyribonucleic acid comprises in 5′- to 3′-direction (in the following order),
      a first recombination sequence,
      one to eight expression cassette(s), whereof one expression cassette encodes one second selection marker, and
      a second recombination sequence,
   wherein the first and the second recombination sequence of the deoxyribonucleic acid are matching the first and the second recombination sequence on the integrated exogenous nucleotide sequence;
c) optionally introducing into or activating in said mammalian cell obtained in step b) a recombinase functional with said first and second recombination sequence (resulting in the exchange of the part of said exogenous nucleotide sequence between the first and second recombination sequence with the part of said deoxyribonucleic acid between the first and second recombination sequence and thereby integration of the latter into the genome said mammalian cell);
d) optionally selecting for cells expressing said second selection marker and producing the proteinaceous compound encoded by the introduced deoxyribonucleic acid,
thereby producing a recombinant mammalian cell comprising a nucleic acid encoding a proteinaceous compound and producing said proteinaceous compound.

In certain embodiments, a method for simultaneous targeted integration of two deoxyribonucleic acids into the genome of a (host) mammalian cell (i.e. a method for producing a recombinant mammalian cell), which comprise nucleic acids encoding a proteinaceous compound and which optionally expresses said proteinaceous compound, comprising the following steps is provided:
a) providing a mammalian cell comprising an exogenous nucleotide sequence integrated at a defined (optionally single) site within a locus of the genome of the mammalian cell, wherein the exogenous nucleotide sequence comprises a first and a second recombination sequence flanking at least one first selection marker, and a third recombination sequence located between the first and the second recombination sequence, and all the recombination sequences are different or/and non-compatible (i.e. these do not result in cross-exchange reactions);
b) introducing into the cell provided in a) a composition of two deoxyribonucleic acids comprising three different recombination sequences and one to eight expression cassettes, wherein
   the first deoxyribonucleic acid comprises in 5′- to 3′-direction (in the following order),
      a first recombination sequence,
      one or more (in one preferred embodiment up to four) expression cassette(s),
      a 5′-terminal part of an expression cassette encoding one second selection marker, and
      a first copy of a third recombination sequence,
   and
   the second deoxyribonucleic acid comprises in 5′- to 3′-direction (in the following order)
      a second copy of the third recombination sequence,
      a 3′-terminal part of an expression cassette encoding the one second selection marker,
      one or more (in one preferred embodiment up to four) expression cassette(s), and
      a second recombination sequence,
   wherein the first to third recombination sequences of the first and second deoxyribonucleic acids are matching the first to third recombination sequence on the integrated exogenous nucleotide sequence,
   wherein the 5′-terminal part and the 3′-terminal part of the expression cassette encoding the one second selection marker when taken together form a functional expression cassette of the one second selection marker;
c) optionally introducing into or activating in said mammalian cell obtained in step b) a recombinase functional with said first, second and third recombination sequence (resulting in the exchange of the part of said exogenous nucleotide sequence between the first and third as well as the part between the third and second recombination sequence with the part of said deoxyribonucleic acids between the first and third as well as the third and second recombination sequence and thereby integration of the latter into the genome said mammalian cell);
d) optionally selecting for cells expressing the second selection marker and optionally producing the proteinaceous product encoded by the introduced deoxyribonucleic acids,
thereby producing a recombinant mammalian cell comprising a nucleic acid encoding said proteinaceous compound.

In order to increase the selection pressure the first selection marker is a negative selection marker, such as, e.g., in certain embodiments, a thymidine kinase from herpes simplex virus (rendering cells sensitive to thymidine analogues, such as 5-iodo-2′-fluoro-2′-deoxy-1-β-D-arabino-furonosyl uracil (FIAU) or ganciclovir) or the diphtheria toxin fragment A from Corynebacterium diphtheria (causing toxicity by inhibiting protein synthesis; for example by phosphoglycerate kinase promoter (PGK)-driven expression of diphtheria toxin A fragment gene). During exchange with the introduced deoxyribonucleic acid, the negative selection marker is removed. This allows the discrimination between correct targeted integration and non-correct random integration.

In certain embodiments of all aspects and embodiments, each of the expression cassettes comprise in 5′-to-3′ direction a promoter, an open reading frame/coding sequence or an RNA gene and a polyadenylation signal sequence, and/or a terminator sequence. In certain embodiments, the open reading frame encodes a polypeptide and the expression cassette comprises a polyadenylation signal sequence with or without additional terminator sequence. In certain embodiments, the expression cassette comprises a RNA gene, the promoter is a type 2 Pol III promoter and a polyadenylation signal sequence or a polyU terminator is present. See, e.g., Song et al. Biochemical and Biophysical Research Communications 323 (2004) 573-578. In certain embodiments, the expression cassette comprises a RNA gene, the promoter is a type 2 Pol III promoter and a polyU terminator sequence.

In certain embodiments of all aspects and embodiments, the open reading frame encodes a polypeptide, the promoter is the human CMV promoter with or without intron A, the polyadenylation signal sequence is the bGH (bovine growth hormone) polyA signal sequence and the terminator is the hGT (human gastrin terminator).

In certain embodiments of all aspects and embodiments the promoter is the human CMV promoter with intron A, the polyadenylation signal sequence is the bGH polyadenylation signal sequence and the terminator is the hGT, except for the expression cassette of the RNA gene and the expression cassette of the selection marker, wherein for the selection marker the promoter is the SV40 promoter and the polyadenylation signal sequence is the SV40 polyadenylation signal sequence and a terminator is absent, and wherein for the RNA gene the promoter is a wild-type type 2 polymerase III promoter and the terminator is a polymerase II or III terminator.

In certain embodiments of all previous aspects and embodiments, the human CMV promoter has the sequence of SEQ ID NO: 28. In certain embodiments, the human CMV promoter has the sequence of SEQ ID NO: 29. In certain embodiments, the human CMV promoter has the sequence of SEQ ID NO: 30.

In certain embodiments of all previous aspects and embodiments, the bGH polyadenylation signal sequence is SEQ ID NO: 31.

In certain embodiments of all previous aspects and embodiments, the hGT has the sequence of SEQ ID NO: 32.

In certain embodiments of all previous aspects and embodiments, the SV40 promoter has the sequence of SEQ ID NO: 33.

In certain embodiments of all previous aspects and embodiments, the SV40 polyadenylation signal sequence is SEQ ID NO: 34.

It has to be pointed out that the current invention does not encompass permanent human cell lines comprising a nucleic acid sequence for the adenoviral gene functions E1A and E1B and concomitantly the nucleic acid sequence for the SV40 large T-antigen or the Epstein-Barr virus (EBV) nuclear antigen 1 (EBNA-1).

Homologous Recombination

In certain embodiments, the targeted integration is mediated by homologous recombination.

Targeted integration by homologous recombination is an established technology in the art. For example, for more than 30 years homologous recombination has been used to introduce specific genetic modifications in a site-specific manner in murine embryonic stem cells (Doetschman, T., et al., Nature 330 (1987) 576-578; Thomas, K. R. and Capecchi, M. R., Cell 51 (1987) 503-512; Thompson, S., et al., Cell 56 (1989) 313-321; Zijlstra, M., et al., Nature 342 (1989) 435-438; Bouabe, H. and Okkenhaug, K., Meth. Mol. Biol. 1064 (2013) 315-336).

In case of the use of homologous recombination for targeted integration, the recombination sequences are sequences homologous to the exogenous nucleic acid sequence and are termed “homology arms”. In this case, the deoxyribonucleic acid introduced into the host cell comprises as first recombination sequence a sequence that is homologous to the sequence 5′ (upstream) to the exogenous nucleic acid sequence (i.e. the landing site) and as second recombination sequence a sequence that is homologous to the sequence 3′ (downstream) to the exogenous nucleic acid sequence. Generally, the targeted integration frequency increases with the length as well as with the isogenicity of the homology arms. Ideally, the homology arms are derived from genomic DNA prepared from the respective host cell.

Nucleases

In certain embodiments, the targeted integration is by homologous recombination mediated by a site-specific nuclease.

In certain embodiments, the site-specific nuclease is selected from Zink finger nuclease (ZFN), transcription activator-like effector nucleases (TALENs) and the clustered regularly interspaced short palindromic repeats (CRISPR)/CRISPRassociated protein-9 nuclease (Cas9) system.

Nuclease-encoding genes can be delivered into cells by plasmid DNA, viral vectors, or in vitro transcribed mRNA. Transfection of plasmid DNA or mRNA can be done by electroporation or cationic lipid-based reagents. Integrase-deficient lentiviral vectors can be used for delivering nucleases into transfection-resistant cell types. AAV vectors can also be used for nuclease delivery.

Recombinases

Recombination systems, such as Cre/LoxP or Flp/FRT, can be used for the exchange of partial nucleic acid sequences between different nucleic acid molecules, the excision of nucleic acid fragments from nucleic acid molecules, or the inversion of parts within a nucleic acid molecule. The result of the action of the recombinase can be permanent using a single on/off-event, it can be for a defined, but limited, period of time, and it can be adjusted to a defined, and thereby, specific cell type or tissue.

Flp-Recombinase

The Flp/FRT site-specific recombination system involves recombination of sequences between the flippase recognition target (FRT) sites by the recombinase flippase (Flp). Flippase originates from Saccharomyces cerevisiae. The sequence of Flp is available, e.g., from UniProt P03870. The 34 bp FRT site has the sequence of GAAGTTCCTATTCtctagaaaGAATAGGAACTTC (SEQ ID NO: 36; central spacer sequence in lower case letters), wherein the Flp-recombinase binds to the inverted 13 bp repeats of GAAGTTCCTATTC (forward SEQ ID NO: 37; inverse SEQ ID NO: 38) flanking the 8 bp central spacer sequence.

Exemplary FRT sites are shown in the following Table (see Branda and Dymecki, Dev. Cell 6 (2004) 7-28):

| name | spacer sequence | SEQ ID NO: |
|---|---|---|
| wild-type | TCTAGAAA | 39 |
| F3 | TTCAAATA | 40 |
| F5 | TTCAAAAG | 41 |

Cre-Recombinase

The Cre/LoxP site-specific recombination system has been widely used in many biological experimental systems. Cre-recombinase is a 38-kDa site-specific DNA recombinase that recognizes 34 bp LoxP sequences. Cre-recombinase is derived from bacteriophage P1 and belongs to the tyrosine family site-specific recombinase. Cre-recombinase can mediate both intra- and intermolecular recombination between LoxP sequences. The canonical LoxP sequence is composed of an 8 bp non-palindromic spacer sequence flanked by two 13 bp inverted repeats. Cre-recombinase binds to the 13 bp repeat thereby mediating recombination within the 8 bp spacer sequence. Cre/LoxP-mediated recombination occurs at a high efficiency and does not require other host factors. If two LoxP sequences are placed in the same orientation on the same nucleotide sequence, Cre-recombinase-mediated recombination will excise the DNA sequence located between the two LoxP sequences as a covalently closed circle. If two LoxP sequences are placed in an inverted/reciprocal orientation with respect to each other on the same nucleotide sequence, Cre-recombinase-mediated recombination will invert the orientation of the DNA sequences located between the two LoxP sequences. If two LoxP sequences are on two different DNA molecules and if one DNA molecule is circular, Cre-recombinase-mediated recombination will result in integration of the circular DNA sequence.

Cre-recombinase can be introduced into or activated inside cells with any known method. For example, using liposome-based gene delivery (WO 93/24640; Mannino and Gould-Fogerite, BioTechniques 6 (1988) 682-691; U.S. Pat. No. 5,279,833; WO 91/06309; Feigner et al., Proc. Natl. Acad. Sci. USA 84 (9871) 7413-7414), or viral vectors such as papilloma viral, retro viral and adeno-associated viral vectors (e.g., Berns et al., Ann. NY Acad. Sci. 772 (1995) 95-104; Ali et al., Gene Ther. 1 (1994) 367-384; Haddada et al., Curr. Top. Microbiol. Immunol. 199 (1995) 297-306; Buchscher et al., J. Virol. 66 (1992) 2731-2739; Johann et al., J. Virol. 66 (1992) 1635-1640; Sommerfelt et al., Virol. 176 (1990) 58-59; Wilson et al., J. Virol. 63 (1989) 2374-2378; Miller et al., J. Virol. 65 (1991) 2220-2224; WO 94/26877; Rosenburg and Fauci in Fundamental Immunology, Third Edition Paul (ed.) Raven Press, Ltd., New York (1993) and the references therein; West et al., Virology 160 (1987) 38-47; U.S. Pat. No. 4,797,368; WO 93/24641; Kotin, Human Gene Therapy 5 (1994) 793-801; Muzyczka, J. Clin. Invest. 94 (1994) 1351; U.S. Pat. No. 5,173,414; Tratschin et al., Mol. Cell. Biol. 5 (1985) 3251-3260; Tratschin et al., Mol. Cell. Biol. 4 (1984) 2072-2081; Hermonat and Muzyczka, Proc. Natl. Acad. Sci. USA 81 (1984) 6466-6470; Samulski et al., J. Virol. 63 (1989) 3822-3828).

For example, a recombinant AAV vector of serotype 2 expressing Cre-recombinase has been described by Li, X., et al. (PLOS ONE 7 (2012) e50063) and Scammell, E., et al. (J. Neurosci. 23 (2003) 5762-5770). Using this rAAV-Cre a very complete recombination of the target LoxP sites could be induced. For rAAV vector-based delivery, see also, Muzyczka, Curr. Top. Microbiol. Immunol. 158 (1992) 97-129; U.S. Pat. No. 4,797,368; WO 91/18088; Samulski, Current Opinion in Genetic and Development 3 (1993) 74-80.

For example, a Cre-recombinase expression plasmid can be used.

For example, Cre-recombinase encoding mRNA can be used.

A large number of functional LoxP sites are known, such as, e.g., Lox511, Lox66, Lox11, Lox76, Lox75, Lox43, Lox44 (see, e.g., Hoess, R., et al., Nucl. Acids Res. 14 (1986) 2287-2300; Albert, H., et al., Plant J. 7 (1995) 649-659).

For example, if Cre-recombinase is used the sequence to be exchanged is defined by the position of the two LoxP sites in the genome as well as in the donor nucleic acid. These LoxP sites are recognized by the Cre-recombinase. Nothing more is required, i.e. no ATP etc.

The Cre/LoxP-system operates in different cell types, like mammals, plants, bacteria and yeast.

Targeted Integration Using Recombinases

In certain embodiments, the targeted integration is by a recombinase mediated cassette exchange reaction (RMCE).

RMCE is an enzymatic process wherein a sequence at the site of integration in the genome is exchanged for a donor nucleic acid. Any recombinase can be used for this process, such as Cre-recombinase, Flp-recombinase, Bxb1-integrase, pSR1-recombinase, or φC31-integrase.

One specific TI method is double recombinase mediated cassette exchange (double RMCE).

Double RMCE is a method for producing a recombinant mammalian cell comprising a deoxyribonucleic acid encoding a proteinaceous compound of interest by recombinase-mediated introduction of two nucleic acid sequences into the host cell's genome at a single locus. After integration, the two nucleic acid sequences are operably linked to each other.

For example, but not by way of limitation, an integrated exogenous nucleotide sequence, i.e. the TI landing site, could comprise two recombination recognition sites (RRSs), while the (donor) nucleic acid sequence comprises two RRSs matching the RRSs on the integrated exogenous nucleotide sequence. Such single-plasmid RMCE strategies allow for the introduction of multiple open reading frames by incorporating the appropriate number of expression cassettes in the respective sequence between the pair of RRSs.

For example, but not by way of limitation, an integrated exogenous nucleotide sequence, i.e. the TI landing site, could comprise three recombination recognition sites (RRSs), e.g., an arrangement where the third RRS (“RRS3”) is present between the first RRS (“RRS1”) and the second RRS (“RRS2”), while a first (donor) nucleic acid comprises two RRSs matching the first and the third RRS on the integrated exogenous nucleotide sequence, and a second (donor) nucleic acid comprises two RRSs matching the third and the second RRS on the integrated exogenous nucleotide sequence. Such double RMCE strategy allows for the introduction of multiple genes by incorporation of the appropriate number of expression cassettes in the respective sequence between each pair of RRSs.

In addition, two selection markers are needed in the two-plasmid RMCE. One selection marker expression cassette is split into two parts. The first (front) nucleic acid could contain the promoter followed by the translation start codon and the RRS3 sequence. The second (back) nucleic acid correspondingly comprises the RRS3 sequence fused to the N-terminus of the selection marker coding sequence, minus the translation start codon (e.g. ATG). Additional nucleotides may need to be inserted between the RRS3 site and the selection marker coding sequence to ensure in frame translation from the fused gene, i.e. operable linkage. Only when both nucleic acids (front and back) are correctly inserted, the full expression cassette of the selection marker will be assembled and, thus, rendering cells resistance to the respective selection agent.

Both single and double RMCE allow for integration of one or more donor DNA molecule(s) into a pre-determined site of a mammalian cell's genome by precise exchange of a DNA sequence present on the donor DNA with a DNA sequence in the mammalian cell's genome where the integration site resides. These DNA sequences are characterized by two heterospecific RRSs flanking i) at least one selection marker or as in certain two-plasmid RMCEs a “split selection marker”; and/or ii) at least one exogenous gene of interest.

RMCE involves a recombinase-catalyzed, double recombination crossover event between the two heterospecific RRSs within the target genomic locus and the donor DNA molecule. Double RMCE is designed to introduce a copy of the DNA sequences from the front- and back-nucleic acid in combination into the pre-determined locus of a mammalian cell's genome. The RMCE procedure can be repeated with multiple DNA sequences.

In certain embodiments, targeted integration is achieved by double RMCE, wherein two different DNA sequences, each comprising at least one expression cassette encoding a part of a proteinaceous compound of interest and/or at least one selection marker or part thereof flanked by two heterospecific RRSs, are both integrated into a pre-determined site of the genome of a mammalian cell suitable for TI. In certain embodiments, targeted integration is achieved by multiple RMCEs, wherein DNA sequences from multiple nucleic acids, each comprising at least one expression cassette encoding a part of a proteinaceous compound of interest and/or at least one selection marker or part thereof flanked by two heterospecific RRSs, are all integrated into a predetermined site of the genome of a mammalian cell suitable for TI. In certain embodiments, the selection marker can be partially encoded on the first nucleic acid (front) and partially encoded on the second nucleic acid (back) such that only the correct integration of both nucleic acids by double RMCE allows for the expression of the selection marker.

For single RMCE and double RMCE the method for the targeted integration of a donor nucleic acid into the genome of a recipient/target cell as well as the method for the simultaneous targeted integration of two donor nucleic acids into the genome of a recipient/target cell as outlined above comprises the additional step of introducing/activating the recombinase.

Thus, in certain embodiments, the recombination sequences are recombination recognition sequences and the method further comprises the following step:
c) introducing or activating
   i) either simultaneously with the introduction of the deoxyribonucleic acid of b); or
   ii) sequentially thereafter
   a recombinase,
   wherein the recombinases recognize the recombination recognition sequences of the first and the second deoxyribonucleic acid; (and optionally wherein the one or more recombinases perform a recombinase mediated cassette exchange).

In certain embodiments, a RRS is selected from the group consisting of a LoxP sequence, a L3 sequence, a 2 L sequence, a LoxFas sequence, a Lox511 sequence, a Lox2272 sequence, a Lox2372 sequence, a Lox5171 sequence, a Loxm2 sequence, a Lox71 sequence, a Lox66 sequence, a FRT sequence, a F3 sequence, a F5 sequence, a Bxb1 attP sequence, a Bxb1 attB sequence, a φC31 attP sequence, and a φC31 attB sequence. If multiple RRSs have to be present, the selection of each of the sequences is dependent on the other insofar as non-identical RRSs are chosen.

In certain embodiments, a RRS can be recognized by a Cre-recombinase. In certain embodiments, a RRS can be recognized by an Flp-recombinase. In certain embodiments, a RRS can be recognized by a Bxb1-integrase. In certain embodiments, a RRS can be recognized by a φC31-integrase. In certain embodiments, a RRS can be recognized by a pSR1-recombinase.

In certain embodiments when the RRS is a LoxP site, the cell requires the Cre-recombinase to perform the recombination.

In certain embodiments when the RRS is a FRT site, the cell requires the Flp-recombinase to perform the recombination.

In certain embodiments when the RRS is a Bxb1 attP or a Bxb1 attB site, the cell requires the Bxb1-integrase to perform the recombination.

In certain embodiments when the RRS is a φC31 attP or a φC31 attB site, the cell requires the φC31-integrase to perform the recombination.

In certain embodiments when the RRS is a recognition site for the pSR1-recombinase of Zygosaccharomyces rouxii, the cell requires the pSR1-recombinase to perform the recombination.

Recombinase-encoding genes can be delivered into cells as DNA, by viral vectors, or as mRNA. Transfection of DNA or mRNA can be done by electroporation or cationic lipid-based reagents. Integrase-deficient lentiviral vectors can be used for delivering recombinases into transfection-resistant cell types. AAV vectors can also be used for recombinase delivery. Recombinase protein can also be introduced by means of nonovesicle.

In certain embodiments of all aspects and embodiments, the recombinase is introduced as mRNA into the cell.

In certain embodiments of all aspects and embodiments, the recombinase is introduced as DNA into the host cell. In certain embodiments, the DNA is a recombinase encoding sequence comprised in an expression cassette.

In certain embodiments of all aspects and embodiments, the recombinase is Cre-recombinase and the Cre-recombinase is introduced as Cre-recombinase encoding mRNA, which encodes a polypeptide that has the amino acid sequence of SEQ ID NO: 07, into the cell.

In certain embodiments of all aspects and embodiments, the Cre-recombinase mRNA encodes a polypeptide comprising the amino acid sequence of SEQ ID NO: 07 and that further comprises at its N- or C-terminus or at both a nuclear localization sequence. In certain embodiments, the Cre-recombinase mRNA encodes a polypeptide that has the amino acid sequence of SEQ ID NO: 07 and further comprises at its N- or C-terminus or at both independently of each other one to five nuclear localization sequences.

In certain embodiments of all aspects and embodiments, the Cre-recombinase encoding mRNA comprises the nucleotide sequence of SEQ ID NO: 08 or a variant thereof with different codon usage. In certain embodiments of all aspects and embodiments, the Cre-recombinase encoding mRNA comprises the nucleotide sequence of SEQ ID NO: 08 or a variant thereof with different codon usage and further comprises at its 5′- or 3′-end or at both a further nucleic acid encoding a nuclear localization sequence. In certain embodiments of all aspects and embodiments, the Cre-recombinase encoding mRNA comprises the nucleotide sequence of SEQ ID NO: 08 or a variant thereof with different codon usage and further comprises at its 5′- or 3′-end or at both independently of each other one to five nucleic acids encoding nuclear localization sequences.

In certain embodiments, a LoxP sequence is a wild-type LoxP sequence. In certain embodiments, a LoxP sequence is a mutant LoxP sequence. Mutant LoxP sequences have been developed to increase the efficiency of Cre-recombinase-mediated integration or replacement. In certain embodiments, a mutant LoxP sequence is selected from the group consisting of a L3 sequence, a 2 L sequence, a LoxFas sequence, a Lox511 sequence, a Lox2272 sequence, a Lox2372 sequence, a Lox5171 sequence, a Loxm2 sequence, a Lox71 sequence, and a Lox66 sequence. For example, the Lox71 sequence has 5 bp mutated in the left 13 bp repeat. The Lox66 sequence has 5 bp mutated in the right 13 bp repeat. Both the wild-type and the mutant LoxP sequences can mediate Cre-recombinase-dependent recombination.

The term “matching RRSs” indicates that a recombination occurs between the two matching RRSs. In certain embodiments, the two matching RRSs are the same. In certain embodiments, both RRSs are wild-type LoxP sequences. In certain embodiments, both RRSs are mutant LoxP sequences. In certain embodiments, both RRSs are wild-type FRT sequences. In certain embodiments, both RRSs are mutant FRT sequences. In certain embodiments, the two matching RRSs are different sequences but can be recognized by the same recombinase. In certain embodiments, the first matching RRS is a Lox71 sequence and the second matching RRS is a Lox66 sequence. In certain embodiments, the first matching RRS is a Bxb1 attP sequence and the second matching RRS is a Bxb1 attB sequence. In certain embodiments, the first matching RRS is a φC31 attB sequence and the second matching RRS is a φC31 attB sequence.

In certain embodiments of all aspects and embodiments, the recombination recognition sites in the double RMCE are L3, 2 L and LoxFas. In certain embodiments, L3 comprises as spacer sequence the sequence of SEQ ID NO: 17, 2 L comprises as spacer sequence the sequence of SEQ ID NO: 18 and LoxFas comprises as spacer sequence has the sequence of SEQ ID NO: 19. In certain embodiments, the first recombination recognition site is L3, the second recombination recognition site is 2 L and the third recombination recognition site is LoxFas.

In certain embodiments of all aspects and embodiments, the expression cassette encoding for a selection marker is located partly 5′ and partly 3′ to the third recombination recognition site, wherein the 5′-located part of said expression cassette comprises the promoter and a translation start-codon and the 3′-located part of said expression cassette comprises the coding sequence without a translation start-codon and a polyA signal sequence.

In certain embodiments of all aspects and embodiments, the 5′-located part of the expression cassette encoding the selection marker comprises a promoter sequence operably linked to a translation start-codon, whereby the promoter sequence is flanked upstream by (i.e. is positioned downstream to) the second, third or fourth, respectively, expression cassette and the start-codon is flanked downstream by (i.e. is positioned upstream of) the third recombination recognition sequence; and the 3′-located part of the expression cassette encoding the selection marker comprises a nucleic acid encoding the selection marker lacking a translation start-codon and is flanked upstream by the third recombination recognition sequence and downstream by a polyA signal sequence and thereafter by the third, fourth, or fifth, respectively, expression cassette.

Any known or future mammalian cell suitable for targeted integration comprising an exogenous nucleic acid (“landing site”) as described herein can be used in the current invention.

In one preferred embodiment of all aspects and embodiments, the mammalian cell comprising an exogenous nucleotide sequence integrated at a single site within a locus of the genome of the mammalian cell is a hamster cell or a human cell, in certain embodiments, a CHO cell.

An exemplary mammalian cell comprising an exogenous nucleotide sequence integrated at a single site within a locus of its genome that is suitable for use in the current invention is a CHO cell or a HEK293 cell or a Per. C6 cell harboring a landing site (=exogenous nucleotide sequence integrated at a single site within a locus of the genome of the mammalian cell) comprising three heterospecific LoxP sites for Cre-recombinase mediated cassette exchange. These heterospecific LoxP sites are, in certain embodiments, L3, LoxFas and 2 L (see e.g. Lanza et al., Biotechnol. J. 7 (2012) 898-908; Wong et al., Nucleic Acids Res. 33 (2005) e147), whereby L3 and 2 L flank the landing site at the 5′-end and 3′-end, respectively, or vice versa, and

LoxFas is located between the L3 and 2 L sites. In certain embodiments of all aspects and embodiments, the landing site further contains a bicistronic unit linking the expression of a selection marker via an IRES to the expression of green fluorescent protein (GFP) allowing to stabilize the landing site by positive selection as well as to select for the absence of the site after transfection and Cre-recombinase-mediated recombination (negative selection). An exemplary GFP has the sequence of SEQ ID NO: 35.

Such a configuration of the landing site as outlined in the previous paragraphs allows for the simultaneous integration of two nucleic acids comprised in different plasmids, a so called front nucleic acid with an L3 and a LoxFas site and a back nucleic acid harboring a LoxFas and an 2 L site. The functional elements of a selection marker gene different from that present in the landing site are distributed between both nucleic acids: promoter and translation start codon are located on the front nucleic acid whereas coding region and poly A signal are located on the back nucleic acid. Only correct Cre-recombinase-mediated integration of both said nucleic acids induces resistance against the respective selection agent.

Generally, a mammalian cell suitable for TI is a mammalian cell comprising an exogenous nucleotide sequence integrated within a locus of its genome, wherein the exogenous nucleotide sequence comprises a first and a second recombination recognition site flanking at least one first selection marker, and a third recombination recognition site located between the first and the second recombination recognition site, and all the recombination recognition sites are different. Said exogenous nucleotide sequence is called a “landing site”.

The presently disclosed subject matter uses a mammalian cell suitable for TI of exogenous nucleotide sequences. In certain embodiments, the mammalian cell suitable for TI comprises an exogenous nucleotide sequence integrated at an integration site in the genome of the mammalian cell. Such a mammalian cell suitable for TI can be denoted also as a “TI host cell”.

In certain embodiments of all aspects and embodiments, the mammalian cell suitable for TI is a hamster cell, a human cell, a rat cell, or a mouse cell comprising a landing site. In certain embodiments, the mammalian cell suitable for TI is a Chinese hamster ovary (CHO) cell, a CHO K1 cell, a CHO K1SV cell, a CHO DG44 cell, a CHO DUKXB-11 cell, a CHO K1S cell, a CHO K1M cell, a human cell, a HEK293 cell, or a Per.C6 cell comprising a respective landing site.

In certain embodiments of all aspects and embodiments, a mammalian cell suitable for TI comprises an integrated exogenous nucleotide sequence, wherein the exogenous nucleotide sequence comprises one or more recombination recognition sites (RRS). In certain embodiments, the exogenous nucleotide sequence comprises at least two RRSs. The RRS can be recognized by a recombinase, for example, a Cre-recombinase, an Flp-recombinase, a Bxb1-integrase, or a φC31-integrase. The RRS can be selected from the group consisting of a LoxP site, a L3 site, a 2 L site, a LoxFas site, a Lox511 site, a Lox2272 site, a Lox2372 site, a Lox5171 site, a Loxm2 site, a Lox71 site, a Lox66 site, a FRT site, a F3 site, a F5 site, a Bxb1 attP site, a Bxb1 attB site, a φC31 attP site, and a φC31 attB site.

In certain embodiments of all aspects and embodiments, the selection marker is independently of each other selected from the group consisting of an aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid. The selection marker(s) can also be a fluorescent protein selected from the group consisting of green fluorescent protein (GFP), enhanced GFP (eGFP), a synthetic GFP, yellow fluorescent protein (YFP), enhanced YFP (eYFP), cyan fluorescent protein (CFP), mPlum, mCherry, tdTomato, mStrawberry, J-red, DsRed-monomer, mOrange, mKO, mCitrine, Venus, YPet, Emerald6, CyPet, mCFPm, Cerulean, and T-Sapphire.

An exogenous nucleotide sequence is a nucleotide sequence that does not originate from a specific cell but can be introduced into said cell by DNA delivery methods, such as, e.g., by transfection, transduction, electroporation, or transformation methods. In certain embodiments of all aspects and embodiments, a mammalian cell suitable for TI comprises at least one exogenous nucleotide sequence integrated at a more integration site in the mammalian cell's genome. In certain embodiments, the exogenous nucleotide sequence is integrated at an integration sites within a specific a locus of the genome of the mammalian cell.

In certain embodiments of all aspects and embodiments, an integrated exogenous nucleotide sequence comprises one or more recombination recognition sites (RRS), wherein the RRS can be recognized by a recombinase. In certain embodiments, the integrated exogenous nucleotide sequence comprises at least two RRSs. In certain embodiments, an integrated exogenous nucleotide sequence comprises three RRSs, wherein the third RRS is located between the first and the second RRS. In certain embodiments, the first and the second RRS are the same and the third RRS is different from the first or the second RRS. In certain embodiments, all three RRSs are different. In certain embodiments, the RRSs are selected independently of each other from the group consisting of a LoxP site, a L3 site, a 2 L site, a LoxFas site, a Lox511 site, a Lox2272 site, a Lox2372 site, a Lox5171 site, a Loxm2 site, a Lox71 site, a Lox66 site, a FRT site, a F3 site, a F5 site, a Bxb1 attP site, a Bxb1 attB site, a φC31 attP site, and a φC31 attB site.

In certain embodiments of all aspects and embodiments, the integrated exogenous nucleotide sequence comprises at least one selection marker. In certain embodiments, the integrated exogenous nucleotide sequence comprises a first, a second and a third RRS, and at least one selection marker. In certain embodiments, a selection marker is located between the first and the second RRS. In certain embodiments, two RRSs flank at least one selection marker, i.e., a first RRS is located 5′ (upstream) and a second RRS is located 3′ (downstream) of the selection marker. In certain embodiments, a first RRS is adjacent to the 5′-end of the selection marker and a second RRS is adjacent to the 3′-end of the selection marker.

In certain embodiments of all aspects and embodiments, a selection marker is located between a first and a second RRS and the two flanking RRSs are different. In certain embodiments, the first flanking RRS is a L3 sequence and the second flanking RRS is a 2 L sequence. In certain embodiments, a L3 sequenced is located 5′ of the selection marker and a 2 L sequence is located 3′ of the selection marker.

In certain embodiments of all aspects and embodiments, the first flanking RRS is a LoxP sequence with wild-type inverted repeats and the second flanking RRS is a LoxP sequence with one mutated inverted repeat. In certain embodiments, the first flanking RRS is a LoxP sequence with a first mutated inverted repeat and the second flanking RRS is a LoxP sequence with a second mutated inverted repeat that is the same or different from the first mutated inverted repeat. In certain embodiments, the first flanking RRS is a LoxP sequence with wild-type inverted repeats and the third RRS is a LoxP sequence with one mutated inverted repeat. In certain embodiments, the second flanking RRS is a LoxP sequence with wild-type inverted repeats and the third RRS is a LoxP sequence with one mutated inverted repeat. In certain embodiments, the first flanking RRS is a LoxP sequence with a first mutated inverted repeat and the third RRS is a LoxP sequence with a second mutated inverted repeat.

In certain embodiments of all aspects and embodiments, the second flanking RRS is a LoxP sequence with a first mutated inverted repeat and the third RRS is a LoxP sequence with a second mutated inverted repeat.

In certain embodiments of all aspects and embodiments, the first flanking RRS is a wild-type FRT sequence and the second flanking RRS is a mutant FRT sequence. In certain embodiments, the first flanking RRS is a first mutant FRT sequence and the second flanking RRS is a second mutant FRT sequence.

In certain embodiments of all aspects and embodiments, the first flanking RRS is a Bxb1 attP sequence and the second flanking RRS is a Bxb1 attB sequence.

In certain embodiments of all aspects and embodiments, the first flanking RRS is a φC31 attP sequence and the second flanking RRS is a φC31 attB sequence.

In certain embodiments of all aspects and embodiments, the integrated exogenous nucleotide sequence comprises a first and a second selection marker, which are flanked by two RRSs, wherein the first selection marker is different from the second selection marker. In certain embodiments, the two selection markers are both independently of each other selected from the group consisting of a glutamine synthetase selection marker, a thymidine kinase selection marker, a HYG selection marker, and a puromycin resistance selection marker. In certain embodiments, the integrated exogenous nucleotide sequence comprises a thymidine kinase selection marker and a HYG selection marker. In certain embodiments, the first selection maker is selected from the group consisting of an aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid, and the second selection maker is selected from the group consisting of a GFP, an eGFP, a synthetic GFP, a YFP, an eYFP, a CFP, an mPlum, an mCherry, a tdTomato, an mStrawberry, a J-red, a DsRed-monomer, an mOrange, an mKO, an mCitrine, a Venus, a YPet, an Emerald, a CyPet, an mCFPm, a Cerulean, and a T-Sapphire fluorescent protein. In certain embodiments, the first selection marker is a glutamine synthetase selection marker and the second selection marker is a GFP fluorescent protein. In certain embodiments, the two RRSs flanking both selection markers are different.

In certain embodiments of all aspects and embodiments, the selection marker is operably linked to a promoter sequence. In certain embodiments, the selection marker is operably linked to an SV40 promoter. In certain embodiments, the selection marker is operably linked to a human Cytomegalovirus (CMV) promoter.

Independent of the method used for the introduction of the donor deoxyribonucleic acid, successfully transfected cells can be selected based on the introduced second selection marker.

It has to be pointed out that when the DNA element, the DNA molecule, or the VA RNA gene according to the current invention is used in combination with recombinase-mediated cassette exchange reactions, different recombinases are used for the RMCE and the RMCI.

For example, the Cre/LoxP-system is used for the recombinase-mediated cassette exchange reaction (RMCE) and the Flp/FRT-system is used for the recombinase-mediated cassette inversion (RMCI) in the DNA element, the DNA molecule, or the

VA RNA according to the current invention. Likewise, the Flp/FRT-system is used for the recombinase-mediated cassette exchange reaction (RMCE) and the Cre/LoxP-system is used for the recombinase-mediated cassette inversion (RMCI) in the DNA element, the DNA molecule, or the VA RNA according to the current invention.

Adeno-Associated Viral Vectors

For a general review of AAVs and of the adenovirus or herpes helper functions see, Berns and Bohensky, Advances in Virus Research, Academic Press., 32 (1987) 243-306. The genome of AAV is described in Srivastava et al., J. Virol., 45 (1983) 555-564. In U.S. Pat. No. 4,797,368 design considerations for constructing recombinant AAV vectors are described (see also WO 93/24641). Additional references describing AAV vectors are West et al., Virol. 160 (1987) 38-47; Kotin, Hum. Gene Ther. 5 (1994) 793-801; and Muzyczka J. Clin. Invest. 94 (1994) 1351. Construction of recombinant AAV vectors described in U.S. Pat. No. 5,173,414; Lebkowski et al., Mol. Cell. Biol. 8 (1988) 3988-3996; Tratschin et al., Mol. Cell. Biol. 5 (1985) 3251-3260; Tratschin et al., Mol. Cell. Biol., 4 (1994) 2072-2081; Hermonat and Muzyczka Proc. Natl. Acad. Sci. USA 81 (1984) 6466-6470; Samulski et al. J. Virol. 63 (1989) 3822-3828.

An adeno-associated virus (AAV) is a replication-deficient parvovirus. It can replicate only in cells, in which certain viral functions are provided by a co-infecting helper virus, such as adenoviruses, herpesviruses and, in some cases, poxviruses such as vaccinia. Nevertheless, an AAV can replicate in virtually any cell line of human, simian or rodent origin provided that the appropriate helper viral functions are present.

Without helper viral genes being present, an AAV establishes latency in its host cell. Its genome integrates into a specific site in chromosome 19 [(Chr) 19 (q13.4)], which is termed the adeno-associated virus integration site 1 (AAVS1). For specific serotypes, such as AAV-2 other integration sites have been found, such as, e.g., on chromosome 5 [(Chr) 5 (p13.3)], termed AAVS2, and on chromosome 3 [(Chr) 3 (p24.3)], termed AAVS3.

AAVs are categorized into different serotypes. These have been allocated based on parameters, such as hemagglutination, tumorigenicity and DNA sequence homology. Up to now, more than 10 different serotypes and more than a hundred sequences corresponding to different clades of AAV have been identified.

The capsid protein type and symmetry determines the tissue tropism of the respective AAV. For example, AAV-2, AAV-4 and AAV-5 are specific to retina, AAV-2, AAV-5, AAV-8, AAV-9 and AAVrh-10 are specific for brain, AAV-1, AAV-2, AAV-6, AAV-8 and AAV-9 are specific for cardiac tissue, AAV-1, AAV-2, AAV-5, AAV-6, AAV-7, AAV-8, AAV-9 and AAV-10 are specific for liver, AAV-1, AAV-2, AAV-5 and AAV-9 are specific for lung.

Pseudotyping denotes a process comprising the cross packaging of the AAV genome between various serotypes, i.e. the genome is packaged with differently originating capsid proteins.

The wild-type AAV genome has a size of about 4.7 kb. The AAV genome further comprises two overlapping genes named rep and cap, which comprise multiple open reading frames (see, e.g., Srivastava et al., J. Viral., 45 (1983) 555-564; Hermonat et al., J. Viral. 51 (1984) 329-339; Tratschin et al., J. Virol., 51 (1984) 611-619). The Rep protein encoding open reading frame provides for four proteins of different size, which are termed Rep78, Rep68, Rep52 and Rep40. These are involved in replication, rescue and integration of the AAV. The Cap protein encoding open reading frame provides four proteins, which are termed VP1, VP2, VP3, and AAP. VP1, VP2 and VP3 are part of the proteinaceous capsid of the AAV particles. The combined rep and cap open reading frames are flanked at their 5′- and 3′-ends by so-called inverted terminal repeats (ITRs). For replication, an AAV requires in addition to the Rep and Cap proteins the products of the genes E1A, E1B, E4orf6, E2A and VA of an adenovirus or corresponding factors of another helper virus.

In the case of an AAV of the serotype 2 (AAV-2), for example, the ITRs each have a length of 145 nucleotides and flank a coding sequence region of about 4470 nucleotides. Of the ITR's 145 nucleotides 125 nucleotides have a palindromic sequence and can form a T-shaped hairpin structure. This structure has the function of a primer during viral replication. The remaining 20, non-paired, nucleotides are denoted as D-sequence.

The AAV genome, harbors three transcription promoters P5, P19, and P40 (Laughlin et al., Proc. Natl. Acad. Sci. USA 76 (1979) 5567-5571) for the expression of the rep and cap genes.

The ITR sequences have to be present in cis to the coding region. The ITRs provide a functional origin of replication (ori), signals required for integration into the target cell's genome, and efficient excision and rescue from host cell chromosomes or recombinant plasmids. The ITRs further comprise origin of replication like-elements, such as a Rep-protein binding site (RBS) and a terminal resolution site (TRS). It has been found that the ITRs themselves can have the function of a transcription promoter in an AAV vector (Flotte et al., J. Biol. Chem. 268 (1993) 3781-3790; Flotte et al., Proc. Natl. Acad. Sci. USA 93 (1993) 10163-10167).

For replication and encapsidation, respectively, of the viral single-stranded DNA genome an in trans organization of the rep and cap gene products are required.

The rep gene locus comprises two internal promoters, termed P5 and P19. It comprises open reading frames for four proteins. Promoter P5 is operably linked to a nucleic acid sequence providing for non-spliced 4.2 kb mRNA encoding the Rep protein Rep78 (chromatin nickase to arrest cell cycle), and a spliced 3.9 kb mRNA encoding the Rep protein Rep68 (site-specific endonuclease). Promoter P19 is operably linked to a nucleic acid sequence providing for a non-spliced mRNA encoding the Rep protein Rep52 and a spliced 3.3 kb mRNA encoding the Rep protein Rep40 (DNA helicases for accumulation and packaging).

The two larger Rep proteins, Rep78 and Rep68, are essential for AAV duplex DNA replication, whereas the smaller Rep proteins, Rep52 and Rep40, seem to be essential for progeny, single-strand DNA accumulation (Chejanovsky & Carter, Virology 173 (1989) 120-128).

The larger Rep proteins, Rep68 and Rep78, can specifically bind to the hairpin conformation of the AAV ITR. They exhibit defined enzyme activities, which are required for resolving replication at the AAV termini. Expression of Rep78 or Rep68 could be sufficient for infectious particle formation (Holscher, C., et al. J. Virol. 68 (1994) 7169-7177 and 69 (1995) 6880-6885).

It is deemed that all Rep proteins, primarily Rep78 and Rep68, exhibit regulatory activities, such as induction and suppression of AAV genes as well as inhibitory effects on cell growth (Tratschin et al., Mol. Cell. Biol. 6 (1986) 2884-2894; Labow et al., Mol. Cell. Biol., 7 (1987) 1320-1325; Khleif et al., Virology, 181 (1991) 738-741).

Recombinant overexpression of Rep78 results in phenotype with reduced cell growth due to the induction of DNA damage. Thereby the host cell is arrested in the S phase, whereby latent infection by the virus is facilitated (Berthet, C., et al., Proc. Natl. Acad. Sci. USA 102 (2005) 13634-13639).

Tratschin et al. reported that the P5 promoter is negatively auto-regulated by Rep78 or Rep68 (Tratschin et al., Mol. Cell. Biol. 6 (1986) 2884-2894). Due to the toxic effects of expression of the Rep protein, only very low expression has been reported for certain cell lines after stable integration of AAV (see, e.g., Mendelson et al., Virol. 166 (1988) 154-165).

The cap gene locus comprises one promoter, termed P40. Promoter P40 is operably linked to a nucleic acid sequence providing for 2.6 kb mRNA, which, by alternative splicing and use of alternative start codons, encodes the Cap proteins VP1 (87 kDa, non-spliced mRNA transcript), VP2 (72 kDa, from the spliced mRNA transcript), and VP3 (61 kDa, from alternative start codon). VP1 to VP3 constitute the building blocks of the viral capsid. The capsid has the function to bind to a cell surface receptor and allow for intracellular trafficking of the virus. VP3 accounts for about 90% of total viral particle protein. Nevertheless, all three proteins are essential for effective capsid production.

It has been reported that inactivation of all three capsid proteins VP1 to VP3 prevents accumulation of single-strand progeny AAV DNA. Mutations in the VP1 amino-terminus (“Lip-negative” or “Inf-negative”) still allows for assembly of single-stranded DNA into viral particles whereby the infectious titer is greatly reduced.

The AAP open reading frame is encoding the assembly activating protein (AAP). It has a size of about 22 kDa and transports the native VP proteins into the nucleolar region for capsid assembly. This open reading frame is located upstream of the VP3 protein encoding sequence.

In individual AAV particles, only one single-stranded DNA molecule is contained. This may be either the “plus” or “minus” strand. AAV viral particles containing a DNA molecule are infectious. Inside the infected cell, the parental infecting single strand is converted into a double strand, which is subsequently amplified. The amplification results in a large pool of double stranded DNA molecules from which single strands are displaced and packaged into capsids.

Adeno-associated viral (AAV) vectors can transduce dividing cells as well as resting cells. It can be assumed that a transgene introduced using an AAV vector into a target cell will be expressed for a long period. One drawback of using an AAV vector is the limitation of the size of the transgene that can be introduced into cells.

Carter et al. have shown that the entire rep and cap open reading frames can be deleted and replaced with a transgene (Carter, B. J., in “Handbook of Parvoviruses”, ed. by P. Tijssen, CRC Press, pp. 155-168 (1990)). Further, it has been reported that the ITRs have to be maintained to retain the function of replication, rescue, packaging, and integration of the transgene into the genome of the target cell.

When cells comprising the respective viral helper genes are transduced by an AAV vector, or, vice versa, when cells comprising an integrated AAV provirus are transduced by a suitable helper virus, then the AAV provirus is activated and enters a lytic infection cycle again (Clark, K. R., et al., Hum. Gene Ther. 6 (1995) 1329-1341; Samulski, R. J., Curr. Opin. Genet. Dev. 3 (1993) 74-80).

E1A is the first viral helper gene that is expressed after adenoviral DNA enters the cell nucleus. The E1A gene encodes the 12S and 13S proteins, which are based on the same E1A mRNA by alternative splicing. Expression of the 12S and 13S proteins results in the activation of the other viral functions E1B, E2, E3 and E4. Additionally, expression of the 12S and 13S proteins force the cell into the S phase of the cell cycle. If only the E1A-derived proteins are expressed, the cell will dye (apoptosis).

E1B is the second viral helper gene that is expressed. It is activated by the E1A-derived proteins 12S and 13S. The E1B gene derived mRNA can be spliced in two different ways resulting in a first 55 kDa transcript and a second 19 kDa transcript. The E1B 55 kDa protein is involved in the modulation of the cell cycle, the prevention of the transport of cellular mRNA in the late phase of the infection, and the prevention of E1A-induced apoptosis. The E1B 19 kDa protein is involved in the prevention of E1A-induced apoptosis of cells.

The E2 gene encodes different proteins. The E2A transcript codes for the single strand-binding protein (SSBP), which is essential for AAV replication

Also the E4 gene encodes several proteins. The E4 gene derived 34 kDa protein (E4orf6) prevents the accumulation of cellular mRNAs in the cytoplasm together with the E1B 55 kDa protein, but also promotes the transport of viral RNAs from the cell nucleus into the cytoplasm.

Generally, to produce recombinant AAV particles, different, complementing plasmids are co-transfected into a host cell. One of the plasmids comprises the transgene sandwiched between the two cis acting AAV ITRs. The missing AAV elements required for replication and subsequent packaging of progeny recombinant genomes, i.e. the open reading frames for the Rep and Cap proteins, are contained in trans on a second plasmid. The overexpression of the Rep proteins results in inhibitory effects on cell growth (Li, J., et al., J. Virol. 71 (1997) 5236-5243).

Additionally, a third plasmid comprising the genes of a helper virus, i.e. E1, E4orf6, E2A and VA from adenovirus, is required for AAV replication.

To reduce the number of required plasmids, Rep, Cap and the adenovirus helper genes may be combined on a single plasmid.

Alternatively, the host cell may already stably express the E1 gene products. Such a cell is a HEK293 cell. The human embryonic kidney clone denoted as 293 was generated back in 1977 by integrating adenoviral DNA into human embryonic kidney cells (HEK cells) (Graham, F. L., et al., J. Gen. Virol. 36 (1977) 59-74). The HEK293 cell line comprises base pair 1 to 4344 of the adenovirus serotype 5 genome. This encompasses the E1A and E1B genes as well as the adenoviral packaging signals (Louis, N., et al., Virology 233 (1997) 423-429).

When using HEK293 cells the missing E2A, E4orf6 and VA genes can be introduced either by co-infection with an adenovirus or by co-transfection with an E2A-, E4orf6- and VA-expressing plasmid (see, e.g., Samulski, R. J., et al., J. Virol. 63 (1989) 3822-3828; Allen, J. M., et al., J. Virol. 71 (1997) 6816-6822; Tamayose, K., et al., Hum. Gene Ther. 7 (1996) 507-513; Flotte, T. R., et al., Gene Ther. 2 (1995) 29-37; Conway, J. E., et al., J. Virol. 71 (1997) 8780-8789; Chiorini, J. A., et al., Hum. Gene Ther. 6 (1995) 1531-1541; Ferrari, F. K., et al., J. Virol. 70 (1996) 3227-3234; Salvetti, A., et al., Hum. Gene Ther. 9 (1998) 695-706; Xiao, X., et al., J. Virol. 72 (1998) 2224-2232; Grimm, D., et al., Hum. Gene Ther. 9 (1998) 2745-2760; Zhang, X., et al., Hum. Gene Ther. 10 (1999) 2527-2537). Alternatively, adenovirus/AAV or herpes simplex virus/AAV hybrid vectors can be used (see, e.g., Conway, J. E., et al., J. Virol. 71 (1997) 8780-8789; Johnston, K. M., et al., Hum. Gene Ther. 8 (1997) 359-370; Thrasher, A. J., et al., Gene Ther. 2 (1995) 481-485; Fisher, J. K., et al., Hum. Gene Ther. 7 (1996) 2079-2087; Johnston, K. M., et al., Hum. Gene Ther. 8 (1997) 359-370).

Thus, cell lines in which the rep gene is integrated and expressed tend to grow slowly or express Rep proteins at very low levels.

A big safety issue is the contamination of the rAAV particle preparation by replication-competent adenoviruses (RCA). RCAs are produced when the vector genome and the adenoviral DNA integrated into the host cell recombine during viral replication by homologous recombination (Lochmueller, H., et al., Hum. Gene Ther. 5 (1994) 1485-1491; Hehir K. M., et al., J. Virol. 70 (1996) 8459-8467). Therefore, HEK 293 cells are not suitable for producing adenoviral vectors for pharmaceutical application.

In order to limit the transgene activity to specific tissues, i.e. to limit the site of integration the transgene can be operably linked to an inducible or tissue specific promoter (see, e.g., Yang, Y., et al. Hum. Gene. Ther. 6 (1995) 1203-1213).

Until today, the main difficulty in the production of rAAV particles is the inefficient packaging of the rAAV vector, resulting in low titers. Packaging has been difficult for several reasons including
preferred encapsidation of wild-type AAV genomes if they are present;
difficulty in generating sufficient complementing functions such as those provided by the wild-type rep and cap genes due to the inhibitory effect associated with the rep gene products;
the limited efficiency of the co-transfection of the plasmid constructs.

All these problems are based on the biological properties of the Rep proteins. Especially the inhibitory (cytostatic and cytotoxic) properties of the Rep proteins as well as the ability to reverse the immortalized phenotype of cultured cells is problematic. Additionally, Rep proteins down-regulate their own expression when the widely used AAV P5 promoter is employed (see, e.g., Tratschin et al., Mol. Cell. Biol. 6 (1986) 2884-2894).

Exemplary Compounds and Compositions According to the Current Invention

Herein are reported novel DNA constructs and methods of using the same. The novel DNA constructs according to the current invention are useful in the simultaneous transcriptional activation of at least two open reading frames using site-specific recombinase technology. The current invention uses a deliberate non-productive arrangement of promoters and open reading frames on coding and template strands of double stranded DNA molecules, which are converted into their productive form by the inversion with a site-specific recombinase.

The principle underlying the technical concept of the current invention is gene expression activation by combined DNA-inversion and operable-linking to a promoter.

One independent aspect of the current invention is a double stranded DNA element comprising a (positively oriented) coding strand and a (negatively oriented) template strand,

characterized in that
the coding strand comprises in positive orientation (i.e. in 5′- to 3′-orientation) in the following order
   a first promoter in positive orientation,
   a first recombinase recognition sequence comprising a mutation in one of the inverted repeats in positive orientation,
   a second promoter in negative orientation (i.e. negative orientation with respect to the coding strand),
   a first polyadenylation signal sequence and/or transcription terminator element in negative orientation (i.e. that is/are inverted with respect to the 5′- to 3′-orientation of the coding strand),
   a first open reading frame in negative orientation and operably linked to the first polyadenylation signal sequence and/or transcription terminator element (i.e. that is inverted with respect to the 5′- to 3′-orientation of the coding strand),
   a second recombinase recognition sequence comprising a mutation in the respective other inverted repeat than the first recombinase recognition sequence and being in negative orientation (i.e. in reciprocal orientation to the first recombinase recognition sequence and inverted with respect to the 5′- to 3′-orientation of the coding strand),
   a second open reading frame in positive orientation, and
   a second polyadenylation signal sequence and/or transcription terminator element in positive orientation and operably linked to the second open reading frame.

One independent aspect of the current invention is a double stranded DNA element comprising in 5′- to 3′-direction in the following order
a first promoter in 5′- to 3′-orientation (i.e. positive orientation),
a first recombinase recognition sequence in 5′- to 3′-orientation comprising a mutation in one of the inverted repeats,
a second promoter in 3′- to 5′-orientation (i.e. negative orientation),
a first polyadenylation signal sequence and/or transcription terminator element in 3′- to 5′-orientation (i.e. that is/are inverted with respect to the 5′- to 3′-orientation of the coding strand),
a first open reading frame in 3′- to 5′-orientation and operably linked to the first polyadenylation signal sequence and/or transcription terminator element,
a second recombinase recognition sequence comprising a mutation in the respective other inverted repeat than the first recombinase recognition sequence and in in 3′- to 5′-orientation (i.e. being in reciprocal orientation to the first recombinase recognition sequence),
a second open reading frame in 5′- to 3′-orientation, and
a second polyadenylation signal sequence and/or transcription terminator element in 5′- to 3′-orientation and operably linked to the second open reading frame.

In certain embodiments of all aspects and embodiments, the incubation of the double stranded DNA element with a recombinase functional with said first and second recombinase recognition sequence results
in the inversion of the sequence between the first and the second recombinase recognition sequence, whereafter the first promoter is operably linked to the first open reading frame and the second promoter is operably linked to the second open reading frame, and
in the generation of a (third) recombinase recognition sequence between the first promoter and the first open reading frame or the second promoter and the second open reading frame following recombination that is no-longer functional with said recombinase.

Thus, the DNA element according to the current invention is non-functional with respect to the transcription of the contained first and second open reading frames. By being non-functional with respect to the transcription of the first and second open reading frame, the DNA element according to the invention can be integrated into genome of a cell without the risk that the comprised open reading frames are expressed already directly after the integration. After introduction into the cell, the open reading frames are only transcribed once a recombinase functional with the recombination recognition sequences of the DNA element, i.e. recognizing the recognition sequences, is activated within or introduced into the cell. Thereby, a recombinase-mediated cassette inversion (RMCI) between the first and second recombinase recognition sequences in the genomically integrated DNA element of the invention is initiated. The RMCI results in an inversion of that part of the DNA element according to the current invention that is located between the two inverted recombinase recognition sequences. Thereby the first promoter becomes operably linked to the first open reading frame and the second promoter becomes operably linked to the second open reading frame. Only thereafter, the first and second open reading frames are transcribed and the respective encoded proteins are expressed. Thus, the DNA element according to the current invention is especially useful for the simultaneous activation of the transcription of two open reading frames within a cell.

The DNA element according to the current invention with transcriptionally inactive open reading frames is depicted schematically in the left part of FIG. 1. The inverted DNA element resulting from RMCI with operably linked promoters and open reading frames, i.e. with transcriptionally active open reading frames, is depicted in the right part of FIG. 1.

Thus, one independent aspect of the current invention is a double stranded DNA element comprising in 5′- to 3′-direction in the following order
a first promoter in 5′- to 3′-orientation (i.e. positive orientation),
a first recombinase recognition sequence in 5′- to 3′-orientation comprising either mutations in both inverted repeats or in none of the inverted repeats,
a first open reading frame in 5′- to 3′-orientation operably linked to the first promoter,
a first polyadenylation signal sequence and/or transcription terminator element in 5′- to 3′-orientation and operably linked to the first open reading frame,
a second promoter in 5′- to 3′-orientation,
a second recombinase recognition sequence comprising either mutations in both inverted repeats if the first recombinase recognition sequence has no mutations in the inverted repeats or no mutations in the inverted repeats if the first recombinase recognition sequence has mutations in both inverted repeats,
a second open reading frame in 5′- to 3′-orientation operably linked to the second promoter, and
a second polyadenylation signal sequence and/or transcription terminator element in 5′- to 3′-orientation and operably linked to the second open reading frame.

The recombinase recognition sequences are maintained in the inverted and thereby activated construct. As the exchange reaction is an enzymatic reaction, a second, i.e. reverse, inversion reaction is possible in case the enzyme is still present/active or reintroduced, as the recombinase recognition sequence, e.g. the LoxP sites, retain their functionality after any exchange. A reverse inversion reaction would result in the transcriptional inactivation of the previously activated open reading frames. The reversibility of the recombinase-mediate cassette inversion depends on the employed recombinase recognition sequences as well as on the used recombinase.

For example, a RMCI reaction catalyzed by Cre-recombinase is a reversible reaction. Thus, cells comprising active Cre-recombinase and LoxP sites in their genome are prone to the intended but also to non-intended inversion events to occur as the recombinase recognition sequences remain functional after each exchange reaction.

Thus, there is a need to control the activity or/and the site of action and/or the reversibility of the recombinase system to prevent secondary, non-intended inversion reactions after the primary, intended inversion reaction has taken place.

Therefore, the DNA element according to the current invention comprises one-sided, mutated recombinase recognition sequences. Thus, each of the recombinase recognition sequences has one wild-type and one mutated inverted repeat. For example, the first recombinase recognition sequence has a mutated left inverted repeat (and a right wild-type repeat) and the second recombinase recognition sequence has a mutated right inverted repeat (and a left wild-type repeat). After RMCI, the activated and productive DNA comprises one recombinase recognition sequence with two wild-type inverted repeats and one recombinase recognition sequence with two mutated inverted repeats. The double mutated recombinase recognition sequence is no longer recognized by the recombinase and thereby the potential back-reaction is prevented. Based on this deliberate design, only a single, i.e. one, RMCI can take place and the transcription is stably activated.

In one preferred embodiment of all aspects and embodiments, the recombinase is Cre-recombinase and the recombinase recognitions sequences are RE- and LE-LoxP sites.

In one preferred embodiment of all aspects and embodiments, the recombinase is Flp-recombinase and the recombinase recognitions sequences are RE- and LE-FRT sites.

Alternatively, phiC31-mediated RMCI can be employed. During such an inversion reaction, the recombination sites are not preserved. In more detail, in contrast to Cre- or FLP-systems, attP and attB sites recombine to create incompatible attL and attR sites, thus preventing successive exchange reactions. Thus, they can be used for one-time, unidirectional RMCI by flanking the sequence to be inverted with inverse attP and attB sites, respectively (see, e.g., Haecker, I., et al., Nat. Sci. Rep. 7 (2017) 43883).

In one preferred embodiment of all aspects and embodiments, the recombinase is phiC31-integrase and the recombinase recognitions sequences are attP and attB. AttP and attB are deemed recombinase recognition sequences with a mutation in one of the repeats according to the current invention as the use of these sequences results in recombinase recognition sequences that are no longer functional after RMCI.

To further increase the advantageous effects of the DNA element according to the current invention the employed promoters can be chosen to be inducible/activatable too. Thus, the transcription of the open reading frames can be turned on after the recombinase mediated inversion only by further specific promoter activation. This results on the one hand in an improved control of the transcription of the open reading frames and on the other hand in the possibility to turn the transcription off again. By the combination of the DNA element according to the current invention and an inducible promoter, potential leakiness of the inducible promoter when used in isolation can be tightened. Inducible systems are known in the art, such as the Tet-on/off-system.

The presently disclosed subject matter not only provides methods for genetic constructs suitable for producing recombinant mammalian cells with inducible transcription of multiple open reading frames but also for stable large-scale production of the respective proteinaceous compound as well. Likewise, recombinant stable producing mammalian cells that have high productivity of the proteinaceous compound of interest can be obtained.

The method according to the current invention can be used with any site-specific recombinases such as Cre-recombinase, Flp-recombinase (recognizing FRT-sites such as GAAGTTCCTATTC-TCTAGAAA-GTATAGGAACTTC (SEQ ID NO: 36)), phiC31-integrase, and Dre-recombinase (recognizing roxP-sites, such as TAACTTTAAATA-ATGCCAAT-TATTTAAAGTTA (SEQ ID NO: 42); Bessem, J. L., et al., Nat. Commun. 10 (2019) 1937) or engineered variants thereof as Tre, Brec 1 and VCre (recognizing LoxP variants such as LoxLTR (ACAACATCCTATT-ACACCCTA-TATGCCAACATGG (SEQ ID NO: 43)) and LoxBTR (AACCCACTGCTTA-AGCCTCAA-TAAAGCTTGCCTT (SEQ ID NO: 44)), or LoxV (TCAATTTCTGAGA-ACTGTCAT-TCTCGGAAATTGA (SEQ ID NO: 45); Sarkar, I., et al., Science 316 (2007) 1912-1915, Karpinski, J., et al., Nat. Biotechnol. 34 (2016) 401-409, Bessem, J. L., et al., Nat. Commun. 10 (2019) 1937) using their respective recombinase-specific LoxP sites, FRT sites, attB/attP sites, and roxP sites, respectively. The only prerequisite is that the used recombinase recognition sequences are non-compatible, i.e. they interact only with a second identical copy and have no detectable promiscuity to closely related sequences.

The method according to the current invention is exemplified in the following using the Cre/LoxP-system, wherein the site-specific recombinase is the Cre-recombinase and the recombination recognition sites are LoxP sites, respectively. This is done in order to exemplify the inventive concept. It can immediately be seen by a person skilled in the art that the inventive concept shown with the Cre/LoxP-system can be applied likewise to other site-specific recombinase systems as listed above, such as the Flp/FRT-system, or the phiC31/att-system, or the Dre/roxP-system. Thus, in the exemplification and definitions provided herein below the term “Cre-recombinase” can be substituted with “Flp-recombinase” or “phiC31-integrase” or “Dre-integrase”, respectively, and the term “LoxP site” can be substituted for the term “FRT site” or “att site” or “roxP site”, respectively.

Depending on the orientation and identity/non-identity of LoxP sites, the recombinase either inverts, excises or replaces the intervening DNA sequence. Thus, in a first mode two LoxP sites are orientated in the same direction. This results in the deletion of the intervening DNA sequence upon interaction with Cre-recombinase leaving an isolated LoxP site behind. In a second mode, the two LoxP sites are orientated in head-to-head direction, i.e. the two LoxP sites are in reciprocal/inverted orientation with respect to each other. In this orientation the interaction with Cre-recombinase results in the inversion of the intervening DNA sequence leaving two LoxP sites behind. During the inversion of the DNA sequence in the second mode the coding-strand and the template-strand between the LoxP sites are exchanged with each other, i.e. what was the coding-strand before the interaction with Cre-recombinase becomes the template-strand after interaction with the Cre-recombinase and vice versa. This process is termed recombinase-mediate cassette inversion (RMCI). In the third mode, two molecules, each comprising a DNA sequence flanked by a first and a second LoxP site oriented in the same direction, whereby one LoxP site on the first molecule and one of the LoxP sites on the second molecule are identical and the second LoxP site on the first molecule is identical to the respective other LoxP site on the second molecule, interact with Cre-recombinase. This interaction results in the exchange of the DNA sequence between the LoxP sites between the two molecules. This process is termed recombinase-mediated-cassette-exchange, or short RMCE.

Variant LoxP sites not compatible with the wild-type LoxP site are known from the art. However, the number of these non-compatible LoxP sites is limited. Some of these sites not to LoxP compatible sites without promiscuity, i.e. without non-specific interaction, are listed in the following Table 1a.

TABLE 1a
Non-compatible LoxP sites.

| | ATAACTTCGTATA-spacer- | |
|---|---|---|
| | TATACGAAGTTAT | |
| site | (SEQ ID NO: 14 + 15) | citation |
| LoxP | ATGTATGC | Langer, S.J., et |
| | (SEQ ID NO: 16) | al., Nucl. Acids |
| | | Res. 30 (2002) |
| | | 3067-3077 |
| Lox5171 | ATGTGTAC | Lee and Saito, |
| | (SEQ ID NO: 21) | Gene 216 |
| | | (1998) 55-65 |
| Lox2272 | AAGTATCC | Lee and Saito, |
| | (SE ID NO: 22) | Gene 216 |
| | | (1998) 55-65 |
| LoxFas | ACAACTTCGTATA/ | Lanza, et al., |
| | TACCTTTC/ | Biotechnol. |
| | TATACGAAGTTGT | J. (2012) 898-908 |
| | (SEQ ID NO: 46) | |
| Lox511 | ATGTATAC | Hoess, et al., |
| | (SEQ ID NO: 20) | Nucl. Acids |
| | | Res. 14 (1986) |
| | | 2287 |
| Loxm3 | TAATACCA | Langer, S.J., |
| | (SEQ ID NO: 24) | et al., Nucl. |
| | | Acids Res. 30 |
| | | (2002) 3067-3077 |
| Loxm7 | AGATAGAA | Langer, S.J., |
| | (SEQ ID NO: 25) | et al., Nucl. |
| | | Acids Res. 30 |
| | | (2002) 3067-3077 |
| L3 | AAGTCTCC | Wong, et al., |
| | (SEQ ID NO: 17) | Nucl. Acids |
| | | Res. 33 (2005) |
| | | e147 |
| — | GTATAGTA | Missirlis, P.I., |
| | (SEQ ID NO: 47) | et al., BMC |
| | | Genomics 7 (2006) |
| | | 73 |
| — | GGCTATAG | Missirlis, P.I., |
| | (SEQ ID NO: 48) | et al., BMC |
| | | Genomics 7 (2006) |
| | | 73 |

FRT sites not compatible with the wild-type FRT site are known from the art. However, the number of these non-compatible FRT sites is limited. Some of such not to FRT compatible sites without promiscuity, i.e. without non-specific interaction, are listed in the following Table 1b.

TABLE 1b
Non-compatible FRT sites.

| | GAAGTTCCTATTC-spacer- | |
|---|---|---|
| | GTATAGGAACTTC | |
| site | (SEQ ID NO: 37 + 38) | citation |
| FRT | TCTAGAAA | McLeod, et al., 1986 |
| | (SEQ ID NO: 39) | |
| F3 | TTCAAATA | Schlake and Bode, 1994 |
| | (SEQ ID NO: 40) | |
| F5 | TTCAAAAG | Schlake and Bode, 1994 |
| | (SEQ ID NO: 41) | |

Single specific non-compatible LoxP sites can be easily found (see Table 1a above). If more than one Cre-lox-based exchange has to be performed in a single nucleic acid, then more than one non-compatible LoxP site is required, i.e. a set comprising two or more non-compatible LoxP sites. That means that each LoxP site in said set has to be non-compatible with all other LoxP sites comprised in said set. Such sets are especially required, if more than one open reading frame is to be selectively activated.

For example, Lee and Saito (Gene 216 (1998) 55-65) synthesized a complete set of 24 LoxP spacer mutants with single-base substitutions and 30 LoxP spacer mutants with double-base substitutions. Out of these, two LoxP spacer mutants, i.e. mutants Lox5171 and Lox2272, were identified, which recombine efficiently with an identical mutant but not with other mutants or wild-type LoxP.

Likewise, Langer, S. J., et al. (Nucl. Acids Res. 30 (2002) 3067-3077) carried out a genetic screen designed to identify novel mutant spacer-containing LoxP sites displaying enhanced non-compatibility with the canonical LoxP site. From Table 1 of Langer et al. it can be seen that it is possible to identify LoxP sets being non-compatible with each other.

TABLE 2
Table 1 of Langer et al.

| lox site | Spacer sequence | Incompatible with |
|---|---|---|
| loxP | GCATACAT | m2, m3, m7, m11 |
| lox511 | GtATACAT | Not tested |
| m2 | tggTttcT | loxP |
| m3 | tggTAtta | loxP, m7 |
| m7 | ttcTAtcT | loxP, m3 |
| m11 | tggTAtcg | loxP |

Lower case letters indicate nucleotides that differ from
the loxP spacer sequence.
(SEQ ID NO: 16, 20, 23, 24, 25, 49)

Missirlis, P. I., et al. (BMC Genomics 7 (2006) 73, A13) performed a high-throughput screen identifying sequence and promiscuity characteristics of the LoxP spacer region in Cre-recombinase mediated recombination. They have identified 31 unique, novel, self-recombining sequences, whereof two had only a single recombination partner.

Exemplary non-compatible LoxP site sets are listed in Table 3.

TABLE 3
Non-compatible LoxP sites sets.

| | spacer sequences | |
|---|---|---|
| | ATAACTTCGTATA-spacer- | |
| site sets | TATACGAAGTTAT | citation |
| Lox5171/ | ATGTGTAC/AAGTATCC | Lee and Saito, |
| Lox2272 | (SEQ ID NO: 21 + 22) | Gene 216 |
| | | (1998) 55-65 |
| Lox2272/ | AGGTATCC/ATGTATAC | Gan and Zhao, |
| Lox511 | (SEQ ID NO: 22 + 20) | Acta. Biochem. |
| | | Biophys. Sin. |
| | | 37 (2005) |
| | | 495-500 |
| LoxP/ | GCATACAT/TACCTTTC | Siegal, et al., |
| LoxFas/ | (also ACAA at 5′- | FEBS Lett. |
| Lox2272 | end of inverted | 499 (2001) |
| | repeats)/GGATACCT | 147-153 |
| | (SEQ ID NO: 16 + 19 + 22) | |
| Lox2272/ | GGATACCT/ATGTATAC/ | Siegal, et al., |
| Lox511-I/ | TACCTTTC | FEBS Lett. |
| LoxFas | (SEQ ID NO: 22 + 20 + 19) | 499 (2001) |
| | | 147-153 |
| LoxP/ | GCATACAT TGGTATTA | Langer, S. J., |
| Loxm3/ | TTCTATCT | et al., Nucl. |
| Loxm7 | (SEQ ID NO: 16 + 24 + 25) | Acids Res. 30 |
| | | (2002) |
| | | 3067-3077 |

Bold nucleotides denote a sequence difference between the
respective publication and Lee and Saito.

Langer, S. J., et al. reported that use of LoxP sites with complementary mutant inverted repeats (Lox66 and Lox71) allowed efficient recombination in trans, whereby a wild-type LoxP site and a defective site with both inverted repeats being mutated was generated. Because the LoxP site with both inverted repeats mutated is no longer an efficient substrate for the recombinase the reaction is driven in one direction.

These complementary mutant inverted repeats contain an altered base-pentett at one of the termini of the repeat sequences. A mutant with the mutations at the terminus of the left inverted repeat is termed LE-mutant. Likewise, that with the mutations at the terminus of the right inverted repeat is termed RE-mutant. The LE-mutant, Lox71, has 5 bp on the 5′-end of the left inverted repeat changed from the wild-type sequence to TACCG (SEQ ID NO: 50) and the RE-mutant, Lox66, has the five 3′-most bases changed to CGGTA (SEQ ID NO: 51). After the recombinase reaction between Lox71 and an inverted Lox66 site located in cis, the resulting LoxP sites are still located in cis enclosing the target DNA sequence, but one of the resulting LoxP site is a doubly mutated site, i.e. with mutations in each terminal sequence, thus, containing both the LE- and RE-inverted repeat mutations. The respective other resulting LoxP site corresponds to the wild-type sequence. Said doubly mutated LoxP site is no longer functional in Cre-recombinase mediated recombinations (see, e.g., Langer et al.; Missirlis et al. both supra).

Different LoxP RE-mutant and LE-mutant sequences are known. Some are given in the following Table 4a.

TABLE 4a
LoxP RE-mutant and LE-mutant sequences.

| mutant | sequence (mutations | |
|---|---|---|
| site | underlined) | citation |
| Lox71 | TACCGTTCGTATA- | Albert, H., et al., |
| (LE) | GCATACAT- | The Plant Journal |
| * | TATACGAAGTTAT | (1995) 649-659 |
| | (SEQ ID NO: 52) | |
| Lox66 | ATAACTTCGTATA- | Albert, H., et al., |
| (RE) | GCATACAT- | The Plant Journal |
| * | TATACGAACGGTA | (1995) 649-659 |
| | (SEQ ID NO: 53) | |
| LoxJTZ17 | ATAACTTCGTATA- | Araki, K., et al., |
| (RE) | GCATACAT- | BMC Biotechnol. |
| * | TATAGCAATTTAT | 10 (2010) 29 |
| | (SEQ ID NO: 54) | |
| LoxKR1 | ATAACTTCGTATA- | Araki, K., et al., |
| (RE) | GCATACAT- | BMC Biotechnol. |
| | TATACCAACTGTT | 10 (2010) 29 |
| | (SEQ ID NO: 55) | |
| LoxKR2 | ATAACTTCGTATA- | Araki, K., et al., |
| (RE) | GCATACAT- | BMC Biotechnol. |
| | TATACCAACTTAA | 10 (2010) 29 |
| | (SEQ ID NO: 56) | |
| LoxKR3 | ATAACTTCGTATA- | Araki, K., et al., |
| (RE) | GCATACAT- | BMC Biotechnol. |
| * | TATACCTTGTTAT | 10 (2010) 29 |
| | (SEQ ID NO: 57) | |
| LoxKR4 | ATAACTTCGTATA- | Araki, K., et al., |
| (RE) | GCATACAT- | BMC Biotechnol. |
| | TATTGCAAGTTAT | 10 (2010) 29 |
| | (SEQ ID NO: 58) | |
| LoxJT15 | AATTATTCGTATA- | WO 2018/190348 |
| (LE) | GCATACAT- | |
| | TATACGAAGTTAT | |
| | (SEQ ID NO: 59) | |

*: with the highest stability after exchange reaction; spacer
in reverse orientation as defined by Hoess et al. (1982).

For example, the RE-mutant and LE-mutant sequences Lox71 and Lox66, or LoxJT15 and LoxJTZ17 can be used as pairs.

Likewise, different FRT RE-mutant and LE-mutant sequences are known. Some are given in the following Table 4b.

TABLE 4b
FRT RE-mutant and LE-mutant sequences.

| Mutant | sequence (mutations | |
|---|---|---|
| site | underlined) | citation |
| LE mutant | GAAGTTCATATTC- | Senecoff, et al., |
| | TCTAGAAA- | 1988 |
| | GTATAGGAACTTC | |
| | (SEQ ID NO: 60) | |
| RE mutant | GAAGTTCCTATTC- | Senecoff, et al., |
| | TCTAGAAA- | 1988 |
| | GTATATGAACTTC | |
| | (SEQ ID NO: 61) | |

Generally, recombination sites containing a (functional) start codon in their sequence on either strand (e.g. LoxP, Lox511, Lox5171, Lox66 or Lox71) must not be placed in a way that after recombination the start codon is located on the coding strand of the 5′ UTR of a gene to be activated. Otherwise, the start codon may repress the translation of the open reading frame. In such a case, the recombination site can be placed (immediately) 3′ of the TATA element of the promoter or between the TATA element and the transcription start site, so that the start codon is not transcribed (silencing of the start codon).

In certain embodiments of all aspects and embodiments, the DNA element of the current invention is combined into dimers, trimers and arrays as long as the used recombinase recognition sites are non-compatible. This is the only requirement when different DNA elements according to the current invention are used in combination. Thereby it is possible to activate transcription of two, four, six and even more open reading frames/genes all at once when the same recombinase is used, or even sequentially when non-compatible recombinases recognition sites of different recombinases are used in each DNA element according to the current invention.

In certain embodiments of all aspects and embodiments, a sequential activation of two, four, six and even more open reading frames/genes is achieved when two or more DNA elements according to the current invention are combined and each DNA element requires for RMCI a different recombinase. This can be achieved by the combination of two of the different site-specific recombinase systems as outlined before, such as, e.g., the combination of the Cre/LoxP-system with the Flp/FRT-system, or the combination of the Cre/LoxP-system with the Dre/roxP-system (see, e.g., Chuang, K., et al., Genes Genom. Genet. 6 (2016) 559-571), or the Cre/LoxP-system with the phiC31-integrase/att-system, or the combination of the Flp/FRT-system with the phiC31-integrase/att-system.

In certain embodiments of all aspects and embodiments, a sequential activation of one, two, three, four, five, six and even more open reading frames/genes is achieved, wherein either one DNA element according to the current invention is used (sequential activation of one or two open reading frames), or two or more DNA elements according to the current invention are combined (sequential activation of two, three, four or more open reading frames), whereby in case of two or more DNA elements each DNA element requires for RMCI a different recombinases, and wherein either the first or the second promoter is an inducible promoter (in case of sequential activation of two open reading frames), or each second promoter is an inducible promoter (in case of sequential activation of two or more open reading frames).

Thus, in certain embodiments of all aspects and embodiments, either the first promoter or the second promoter is an inducible promoter. In certain embodiments, the inducible promoter is selected from the group of inducible promoters comprising a tetracycline-controlled promoter, a cumate-controlled promoter, an FKBP12-mTOR-controlled promoter, a rapamycin-controlled promoter, an FKCsA-controlled promoter, an abscisic acid-controlled promoter, a tamoxifen-controlled promoter, and a riboswitch-controlled promoter (FKCsA=heterodimer of FK506 and cyclosporine A).

For a review of inducible promoters see, e.g., Kallunki, T., et al., Cells 8 (2019) 796.

In certain embodiments of all aspects and embodiments, a sequential activation of one, two, three, four, five, six and even more open reading frames/genes is achieved, wherein either one DNA element according to the current invention is used (sequential activation of one or two open reading frames), or two or more DNA elements according to the current invention are combined (sequential activation of two, three, four or more open reading frames), whereby in case of two or more DNA elements each DNA element requires for RMCI a different recombinases, and wherein either the first or the second promoter is a repressible promoter (in case of sequential activation of two open reading frames), or each second promoter is an repressible promoter (in case of sequential activation of two or more open reading frames).

Thus, in certain embodiments of all aspects and embodiments, either the first promoter or the second promoter is a repressible promoter. In certain embodiments, the repressible promoter is selected from the group of repressible promoters comprising a tetracycline-controlled promoter, a GAL4/UAS-controlled promoter, and a LexA/lexAop-controlled promoter.

To allow for even more combinations, constitutive, inducible and repressible promoters can be combined. If for example tetracycline-dependent inducible and repressible promoters are combined, by the addition of tetracycline one promoter is silenced and the other is activated, allowing for a switching of the transcription of different open reading frames.

In FIG. 2 the combination of two DNA elements according to the current invention is shown. The first DNA element comprises a first recombinase recognition sequence (RRS1) with mutation in the left inverted repeat in forward orientation, a first open reading frame (SG1) in reverse orientation operably linked to a first polyadenylation signal sequence also in reverse orientation, a second recombinase recognition sequence (RRS2) with mutation in the right inverted repeat in backward orientation, which is compatible with RRS1, and an open reading frame (SG2) in forward orientation operably linked to a second polyadenylation signal sequence. The second DNA element comprises a third recombinase recognition sequence (RRS3) with mutation in the left inverted repeat in forward orientation, which is non-compatible with RRS1 and RRS2, a third open reading frame (SG3) in reverse orientation operably linked to a third polyadenylation signal sequence, a fourth recombinase recognition sequence (RRS4) with mutation in the right inverted repeat in backward orientation, which is non-compatible with RRS1 and RRS2 and compatible with RRS3, and a forth open reading frame (SG4) operably linked to a fourth polyadenylation signal sequence.

If all RRSs are recognized by a single, i.e. the same, recombinase, then upon incubation therewith two inversion reactions take place, i.e. the DNA fragment between RRS1 and RRS2 as well as the DNA fragment between RRS3 and RRS4 is inverted. Thereby all four open reading frames become operably linked to their respective promoters and are transcribed. The respective exchange reaction is shown in FIG. 3. For example, if Cre-recombinase is used, the non-compatible RRSs pairs Lox71/Lox66 and L3-LE/L3-RE, respectively, can be used.

If RRS1 and RRS2 are recognized by a first recombinase and RRS3 and RRS4 are recognized by a second recombinase, then upon incubation with the first recombinase only one inversion reaction takes place, i.e. the DNA fragment between RRS1 and RRS2 is inverted, whereas the DNA fragment between RRS3 and RRS4 is maintained. Thereby only two open reading frames become operably linked to their respective promoters and are transcribed. If after the first recombinase the respective second recombinase is introduced into the respective cell, also the DNA fragment between RRS3 and RRS4 is inverted and the respective open reading frames become activated. The respective exchange reaction is shown in FIG. 4. For example, the first recombinase can be Cre-recombinase and RRS1/RRS2 are LoxP sites, the second recombinase can be phiC31-integrase, and RRS3/RRS4 are attP and attB.

If at least one of the promoters is an inducible promoter, the transcription of the thereto operably linked open reading frame requires after RMCI additionally the presence of the respective inducer, or if at least one of the promoters is a repressible promoter, the transcription of the thereto operably linked open reading frame can be suppressed after RMCI by the addition of the respective repressor.

Recombinant AAV Particles

For the generation of recombinant AAV particles, expression of the Rep and Cap proteins, the helper proteins E1A, E1B, E2A and E4orf6 as well as the adenoviral VA RNA in a single mammalian cell is required. Especially the expression of the Rep proteins has negative impact on the growth and viability of mammalian cells. These drawbacks can be overcome by employing the DNA element according to the current invention. Exemplary designs are outlined below and are shown in FIGS. 5, 6 and 7, wherein one or two DNA elements according to the current invention is/are used in combination. The helper proteins E1A, E1B, E2A and E4orf6 can be expressed using any promoter as shown by Matsushita et al. (Gene Ther. 5 (1998) 938-945), especially the CMV IE promoter. Thus, in the following any promoter can be used.

E1A, E1B, E2A, E4orf6 Open Reading Frames

Thus, one independent aspect of the current invention is a (double stranded) DNA (molecule) (for the production of recombinant adeno-associated virus vectors or particles) comprising
a) the E1A open reading frame and the E1B open reading frame; and
b) the E2A open reading frame and the E4orf6 open reading frame;
characterized in that the first and the second open reading frame of a) or b) are contained in a double stranded DNA element (according to the current invention) comprising a (positively oriented) coding strand and a (negatively oriented) template strand,
wherein the coding strand comprises in 5′- to 3′-orientation in the following order
   a first promoter,
   a first recombinase recognition sequence comprising a mutation in the left inverted repeat,
   a second promoter that is inverted with respect to the coding strand (in inverted orientation),
   a first polyadenylation signal sequence and/or transcription termination element that is/are inverted with respect to the coding strand and that is operably linked to the first open reading frame,
   the first open reading frame of a) or b) that is inverted with respect to the coding strand (in inverted orientation),
   a second recombinase recognition sequence comprising a mutation in the right inverted repeat and in reciprocal orientation to the first recombinase recognition sequence,
   the second open reading frame of a) if the first open reading frame is of a) or the second open reading frame of b) if the first open reading frame is of b),
   a second polyadenylation signal sequence and/or transcription termination element that is operably linked to the second open reading frame.

In certain embodiments of all aspects and embodiments, the respective other open reading frames are within an expression cassette, i.e. operably linked to a promoter and a polyadenylation signal sequence and/or transcription termination element.

FIGS. 9 and 10 show a scheme of the above aspect a) before RMCI (FIG. 9) and after RMCI (FIG. 10).

FIGS. 11 and 12 show a scheme of the above aspect b) before RMCI (FIG. 11) and after RMCI (FIG. 12).

The sequences of the recombination recognition sites in the DNA element according to the current invention need to have a specific orientation with respect to each other. The first recombination recognition site is in forward orientation and the second recombination recognition site is in inverted/reverse orientation with respect to the first recombination recognition site.

For example, in case of a LoxP site with the following sequence in 5′-to-3′-orientation as on the coding strand/positive strand/forward strand:

5′-ataacttcgtata-atgtatgc-tatacgaagttat-3′

the inverted sequence to be placed in the coding strand, i.e. in 5′- to 3′-orientation, is obtained by replacing each nucleotide with its complementary base and starting from the 3′-end of the original sequence, which results in the following inverted coding strand sequence:

5′-ataacttcgtata-gcatacat-tatacgaagttat-3′.

Likewise, the other inverted sequences, which are combined in the DNA element according to the current invention, can be obtained. An exemplary DNA element according to the current invention has, thus, the following sequence on the coding strand:
1^{st}-promoter in normal orientation—
5′-ataacttcgtata-atgtatgc-tatacgaagttat-3′ (1^{st} recombinase recognition sequence in normal orientation)—
2^{nd} promoter in inverted orientation—
1^{st} polyA/terminator sequence in inverted orientation—
1^{st} open reading frame in inverted orientation—
5′-ataacttcgtata-gcatacat-tatacgaagttat-3′ (2nd recombinase recognition sequence in inverted orientation)—
2^{nd} open reading frame (in normal orientation)—
2^{nd} polyA/terminator sequence in normal orientation.

Further, one independent aspect of the current invention is a (double stranded) DNA (molecule) (for the production of recombinant adeno-associated virus vectors or particles) comprising
a) the E1A open reading frame and the E1B open reading frame; and
b) the E2A open reading frame and the E4orf6 open reading frame;
characterized in that the first and the second open reading frame of a) are contained in a double stranded DNA element (according to the current invention) and the first and the second open reading frame of b) are contained in a double stranded DNA element (according to the current invention) (i.e. the DNA comprises two of said DNA elements according to the current invention) each double stranded DNA element comprises a (positively oriented) coding strand and a (negatively oriented) template strand,
wherein the coding strand comprises in 5′- to 3′-orientation in the following order
   a first promoter,
   a first recombinase recognition sequence comprising a mutation in the left inverted repeat,
   a second promoter that is inverted with respect to the coding strand (in inverted orientation),
   a first polyadenylation signal and/or transcription termination element that is/are inverted with respect to the coding strand and that is operably linked to the first open reading frame,
   the first open reading frame of a) or b) that is inverted with respect to the coding strand (in inverted orientation),
   a second recombinase recognition sequence comprising a mutation in the right inverted repeat and in reciprocal orientation to the first recombinase recognition sequence,
   the second open reading frame of a) if the first open reading frame is of a) or the second open reading frame of b) if the first open reading frame is of b), and
   a second polyadenylation signal and/or transcription termination element that is operably linked to the second open reading frame.

In each case, the incubation of the double stranded DNA molecule with a recombinase functional with said first and second recombinase recognition sequence results
in the inversion of the sequence between the first and the second recombinase recognition sequence, whereafter the first promoter is operably linked to the first open reading frame and the second promoter is operably linked to the second open reading frame, and
in the generation of a (third) recombinase recognition sequence located between the first promoter and the first open reading frame following recombination that is no longer functional with said recombinase.

In the above two aspects likewise the first recombinase recognition sequence can comprise a mutation in the right inverted repeat and the second recombinase recognition sequence can comprise a mutation in the left inverted repeat. This will result in the generation of a recombinase recognition sequence located between the second promoter and the second open reading frame following recombination that is no longer functional with said recombinase.

Temporal expression of a recombinase, e.g. the Cre-recombinase can be achieved by using either an inducible promoter driving the expression of the recombinase gene, or by the introduction of recombinase encoding mRNA etc. An exemplary inducible Cre-recombinase expression system was reported by Carter, Z. and Delneri, D. (Yeast 27 (2010) 765-775). Therein the expression of the Cre-recombinase was induced in the transformants by exposing them to galactose (YPGal) for some hours.

The coding sequences of E1A and E1B (open reading frames) are in certain embodiments of all aspects and embodiments derived from a human adenovirus, such as, e.g., in particular of human adenovirus serotype 2 or serotype 5. An exemplary sequence of human Ad5 (adenovirus serotype 5) can be found in GenBank entries X02996, AC_000008 and that of an exemplary human Ad2 in GenBank entry AC_000007. In certain embodiments of all aspects and embodiments, nucleotides 505 to 3522 comprise the nucleic acid sequences encoding E1A and E1B of human adenovirus serotype 5. Plasmid pSTK146 as reported in EP 1 230 354 B 1, as well as plasmids pGS119 and pGS122 as reported in WO 2007/056994, can also be used a source for the E1A and E1B open reading frames.

Rep/Cap Open Reading Frames

The principle of gene activation by combined DNA-inversion and operable-linking to a promoter can also be used to conditionally activate the rep and cap open reading frames.

Except for the P5 promoter, the promoters, which are driving the rep and cap open reading frame expression are located within the Rep-polypeptide coding sequence. Thus, for the conditional activation of the rep and cap open reading frames by recombinase-mediated sequence inversion and concomitant operable-linking to a promoter, one of the non-compatible recombinase recognition sequences has to be located between the P5 promoter and the rep open reading frame and the other non-compatible recombinase recognition sequence has to be located between the cap open reading frame and the polyadenylation signal. This is schematically shown in the left sketch of FIG. 7.

Thus, one independent aspect of the current invention is a (double stranded) DNA (molecule) (for the production of recombinant adeno-associated virus vectors or particles) comprising a double stranded DNA element (according to the current invention), comprising a (positively oriented) coding strand and a (negatively oriented) template strand,
wherein the coding strand comprises in 5′- to 3′-orientation in the following order
   a first promoter, in one preferred embodiment the adeno-associated viral promoter P5 or a functional fragment thereof or a variant thereof,
   a first recombinase recognition sequence comprising a mutation in the left inverted repeat,
   the rep and cap open reading frames including further promoters for the expression of the Rep and Cap proteins, which are inverted with respect to the coding strand (in inverted orientation),
   a second recombinase recognition sequence comprising a mutation in the right inverted repeat and in inverted/reciprocal orientation to the first recombinase recognition sequence, and
   a polyadenylation signal.

Another independent aspect of the current invention is a (double stranded) DNA (molecule) (for the production of recombinant adeno-associated virus vectors or particles) comprising a double stranded DNA element (according to the current invention), comprising a (positively oriented) coding strand and a (negatively oriented) template strand,
wherein the coding strand comprises in 5′- to 3′-orientation in the following order
   a first promoter, in one preferred embodiment the adeno-associated viral promoter P5 or a functional fragment thereof or a variant thereof,
   a first recombinase recognition sequence comprising a mutation in the left inverted repeat,
   a second promoter that is inverted with respect to the coding strand (in inverted orientation), in one preferred embodiment the adeno-associated viral promoter P19 or a functional fragment thereof or a variant thereof,
   a first polyadenylation signal and/or transcription termination element that is/are inverted with respect to the coding strand,
   a coding sequence, which encodes either exclusively the Rep78 protein or exclusively the Rep68 protein, but not both, wherein the internal P40 promoter is inactivated and splice donor and acceptor sites are removed, and which is inverted with respect to the coding strand (in inverted orientation),
   a second recombinase recognition sequence comprising a mutation in the right inverted repeat and in inverted/reciprocal orientation to the first recombinase recognition sequence, and
   the Rep52/Rep40 and Cap gene including a common polyadenylation signal.

FIGS. 13 and 14 show a scheme of the above aspect before RMCI (FIG. 13) and after RMCI (FIG. 14). See also FIG. 7, middle sketch.

Another independent aspect of the current invention is a (double stranded) DNA (molecule) (for the production of recombinant adeno-associated virus vectors and particles) comprising a double stranded DNA element (according to the current invention), comprising a (positively oriented) coding strand and a (negatively oriented) template strand,
wherein the coding strand comprises in 5′- to 3′-orientation in the following order
   a first promoter, in one preferred embodiment the adeno-associated viral promoter P5 or a functional fragment thereof or a variant thereof,
   a first recombinase recognition sequence comprising a mutation in the left inverted repeat,
   a second promoter that is inverted with respect to the coding strand (in inverted orientation), in one preferred embodiment the adeno-associated viral promoter P19 or a functional fragment thereof or a variant thereof,
   a first polyadenylation signal and/or transcription termination element in that is inverted (in sequence) with respect to the coding strand (direction) (i.e. is in inverted/negative orientation) and that is operably linked to the Rep78 or Rep68 coding sequence,
   a coding sequence, which encodes either exclusively the Rep78 protein or exclusively the Rep68 protein, but not both, wherein the internal P40 promoter is inactivated and splice donor and acceptor sites are removed, and which is inverted with respect to the coding strand (in inverted orientation),
   a second recombinase recognition sequence, which comprises a mutation in the right inverted repeat, and which is in reciprocal/inverted orientation with respect to the first recombinase recognition sequence,
   the Rep52 open reading frame including a polyadenylation signal sequence, i.e. a polyadenylation signal operably linked to said open reading frame, and
   optionally a third promoter, a cap open reading frame and a polyadenylation and/or terminator sequence, wherein all are operably linked.

See also FIG. 7, right sketch.

In each of the above aspects the incubation of the double stranded DNA molecule with a recombinase functional with said first and second recombinase recognition sequence and/or said third and fourth recombinase recognition sequence, respectively, results
in the inversion of the sequence between the first/third and the second/fourth recombinase recognition sequence, whereafter the first/third promoter is operably linked to the first/third open reading frame and the second/fourth promoter is operably linked to the second/fourth open reading frame, and
in the generation of a recombinase recognition sequence between the first/third promoter and the first/third open reading frame following recombination that is no-longer functional with said recombinase.

In the above three aspects likewise the first recombinase recognition sequence can comprising a mutation in the right inverted repeat and the second recombinase recognition sequence can comprising a mutation in the left inverted repeat. This will result in the generation of a recombinase recognition sequence located between the second promoter and the second open reading frame following recombination that is no longer functional with said recombinase.

Temporal expression of a recombinase, e.g. the Cre-recombinase can be achieved either by using an inducible promoter driving the expression of the recombinase gene, or by the introduction of recombinase encoding mRNA etc. An exemplary inducible Cre-recombinase expression system was reported by Carter, Z. and Delneri, D. (Yeast 27 (2010) 765-775). Therein the expression of the Cre-recombinase was induced in the transformants by exposing them to galactose (YPGal) for some hours.

Adenoviral VA RNA Gene

The principle of gene activation by combined DNA-inversion and operable-linking to a promoter can also be used to conditionally activate the adenoviral VA RNA gene transcription.

Adenoviral VA RNA genes are driven by type 2 polymerases III promoters, which comprise two intragenic elements, A-box and B-box. Snouwaert et al. (Nucl. Acids Res. 15 (1987) 8293-8303) identified mutants of the VA RNAI B-box that completely abrogate promoter activity. These mutations are unlikely to affect binding of VA RNAI to PKR and related functions (Clark, K. R., et al., Hum. Gene Ther. 6 (1995) 1329-1341).

A further aspect of the invention is a novel adenoviral VA RNA gene. The adenoviral VA RNA gene according to the current invention enables Cre-recombinase mediated gene activation by inversion. In the adenoviral VA RNA according to the current invention, the adenoviral VA RNA gene can be driven by any promoter with a precise transcription start site together with a LoxP site introduced into the non-coding, i.e. regulatory elements of the adenoviral VA RNA.

The current inventors have found that a TATA box can be integrated into the 8 bp spacer of a LoxP site resulting in a specifically engineered novel LoxP site. Said novel LoxP spacer sequence AGTTTATA (SEQ ID NO: 01) is denoted as Lx. This novel spacer sequence can be combined with any known inverted repeat sequences, such as the wild-type LoxP inverted repeat sequences of SEQ ID NO: 14 and 15 (=SEQ ID NO: 14+SEQ ID NO: 01+SEQ ID NO: 15), as well as inverted repeat sequences comprising the LE- and RE-mutant sequence of SEQ ID NO: 50 and 51 (=SEQ ID NO: 03 and 05), in forward as well as inverted (inv) form (=SEQ ID NO: 14+SEQ ID NO: 02+SEQ ID NO: 15):

Lx
ataacttcgtata-agtttata-tatacgaagttat
Lx (inv)
ataacttcgtata-tataaact-tatacgaagttat
Lx-LE
taccgttcgtata-agtttata-tatacgaagttat
Lx-RE
ataacttcgtata-agtttata-tatacgaacggta

Below a sequence alignment of the LoxP site (1), the Lx-LE site according to the current invention (with mutated left inverted repeat) (2), and an exemplary TATA box (3) is shown (underlined TATA box, spacer sequence in bold):

| | |
|---|---|
| (1) | ATAACTTCGTATAATGTATGCTATACGAAGTTAT |
| (2) | TACCGTTCGTATAAGTTTATATATACGAAGTTAT |
| (3) | TTTATATAT |

It can be seen that the Lx-LE site according to the invention leaves the TATA box unchanged, comprises the mutant left repeat (LE), the wild-type right repeat and the novel Lx spacer sequence.

Thus, one aspect of the current invention is a Cre-recombinase recognition sequence

Lx-LE of SEQ ID NO: 30
(TACCGTTCGTATAAGTTTATATATACGAAGTTAT).

Thus, an independent aspect of the invention is the LoxP site AGTTTATA (SEQ ID NO: 01 forward orientation; SEQ ID NO: 02 reverse orientation).

In certain embodiments of all aspects and embodiments, the spacer sequence of SEQ ID NO: 01 or SEQ ID NO: 02 is combined with a wild-type left inverted repeat and a wild-type right inverted repeat. This Cre-recombinase recognition sequence has in forward orientation the direct combination of the sequences of SEQ ID NO: 14+SEQ ID NO: 01+SEQ ID NO: 15 and in reverse orientation the direct combination of the sequences of SEQ ID NO: 14+SEQ ID NO: 02+SEQ ID NO: 15.

In certain embodiments of all aspects and embodiments, the spacer sequence of SEQ ID NO: 01 or SEQ ID NO: 02 is combined with a mutated left inverted repeat and a wild-type right inverted repeat. This Cre-recombinase recognition sequence is denoted as Lx-LE and has in forward orientation the sequence of SEQ ID NO: 03 and in reverse orientation the sequence of SEQ ID NO: 04.

In certain embodiments of all aspects and embodiments, the spacer sequence of SEQ ID NO: 01 or SEQ ID NO: 02 is combined with a mutated right inverted repeat and a wild-type left inverted repeat. This Cre-recombinase recognition sequence is denoted as Lx-RE and has in forward orientation the sequence of SEQ ID NO: 05 and in reverse orientation the sequence of SEQ ID NO: 06.

A further independent aspect of the current invention is the use of the Cre-recombinase recognition sequence of SEQ ID NO: 03 in the transcription of the adenoviral VA RNA gene.

A further independent aspect of the current invention is a novel adenoviral VA RNA gene. The adenoviral VA RNA gene according to the current invention enables Cre-recombinase mediated gene activation by inversion. In the adenoviral VA RNA according to the current invention, the adenoviral VA RNA gene transcription can be driven by any promoter with a precise transcription start site together with a LoxP site introduced into the non-coding, i.e. regulatory, elements of the adenoviral VA RNA.

This aspect of the invention is shown in FIG. 16.

The viral associated RNA (VA RNA) is a non-coding RNA of adenovirus (Ad), regulating translation. The adenoviral genome comprises two independent copies: VAI (VA RNAI) and VAII (VA RNAII). Both are transcribed by RNA polymerase III (see, e.g., Machitani, M., et al., J. Contr. Rel. 154 (2011) 285-289).

The structure, function, and evolution of adenovirus-associated RNA using a phylogenetic approach was investigated by Ma, Y. and Mathews, M. B. (J. Virol. 70 (1996) 5083-5099). They provided alignments as well as consensus VA RNA sequences based on 47 known human adenovirus serotypes. Said disclosure is herewith incorporated by reference in its entirety into the current application.

VA RNAs, VAI and VAII, are consisting of 157-160 nucleotides (nt).

Depending on the serotype, adenoviruses contain one or two VA RNA genes. VA RNAI is believed to play the dominant pro-viral role, while VA RNAII can partially compensate for the absence of VA RNAI (Vachon, V. K. and Conn, G. L., Virus Res. 212 (2016) 39-52).

The VA RNAs are not essential, but play an important role in efficient viral growth by overcoming cellular antiviral machinery. That is, although VA RNAs are not essential for viral growth, VA RNA-deleted adenovirus cannot grow during the initial step of vector generation, where only a few copies of the viral genome are present per cell, possibly because viral genes other than VA RNAs that block the cellular antiviral machinery may not be sufficiently expressed (see Maekawa, A., et al. Nature Sci. Rep. 3 (2013) 1136).

The A- and B-boxes, which constitute the internal control regions (or promoter) for RNA polymerase III, have been defined experimentally for adenoviral serotype 2 (Ad 2) VA RNAI. These are well conserved. All of the VA RNAs have both boxes at similar positions. The B-box homology is very high. The A-boxes, located 34 to 40 nt upstream of the B-box, are slightly less homologous in some of the VA RNAs. A pair of mutually complementary tetranucleotides, CCGG (SEQ ID NO: 77) and (U/C)CCGG (SEQ ID NO: 78), that forms part of the apical stem of the VA RNA is reasonably well conserved in VA RNA sequences. The first CCGG, which includes the first two bases of the B-box, is invariant. All of the VA RNA genes but one have sequences in the 5′ half homologous to the tRNA transcription initiation elements, the A- and B-box consensus sequences RRYNNARYGG (SEQ ID NO: 79) and GWTCRANNC (SEQ ID NO: 80), respectively. The A-box homology in the VA RNAII genes is generally weaker than that in the VA RNAI genes, in accord with the finding that the A-box is less important for VA RNA transcription than the B-box. At the end of the VA RNA coding sequences is a run of T residues flanked by the nucleotides C and G, typical of polymerase III termination sites. The number of thymidins varies from a minimum of 4 to more than 10, and A residues are absent for at least 3 nt on either side of the T-rich run (except in Ad 12 and Ad 18, which have A residues in the middle of very long T runs) (Ma, Y. and Mathews, M. B., J. Virol. 70 (1996) 5083-5099).

The B-box sequences of the VA RNAI and VA RNAII have been found to be essential for the activity of the internal polymerase-III promoter.

Maekawa, A., et al. (Nature Sci. Rep. 3 (2013) 1136) reported efficient production of adenovirus vector lacking genes of virus-associated RNAs that disturb cellular RNAi machinery, wherein HEK293 cells that constitutively and highly express flippase recombinase were infected to obtain VA RNA-deleted adenovirus by FLP recombinase-mediated excision of the VA RNA locus.

The human adenovirus 2 VA RNAI (nucleotides 10586-10810 of GenBank entry AC_000007) sequence is shown in SEQ ID NO: 81; that of the G58T/G59T/C68A (consecutive residue numbering) in SEQ ID NO: 82. The human adenovirus 5 VA RNAI (nucleotides 10579-10820 of GenBank entry AC_000008) sequence is shown in SEQ ID NO: 83; that of the human adenovirus 5 VA RNAI and VA RNAII in SEQ ID NO: 84.

Hahn, S. (Nat. Struct. Mol. Biol. 11 (2004) 394-403) and Revyakin, A., et al. (Gen. Devel. 26 (2012) 1691-1702) reported about the structure and mechanism of the RNA Polymerase II transcription machinery and Nikitina, T. V. and Tishchenko, L. I. (Mol. Biol. 39 (2005) 161-172) reviewed RNA Polymerase III transcription machinery. These are summarized in the following.

Transcription, that is, RNA synthesis on a DNA template, is performed by DNA-dependent RNA polymerases (Pols, [EC 2.7.7.6]). Beside the RNA polymerase additional factors, termed general transcription factors (GTF), are involved. These are required for recognition of the promoter sequences, the response to regulatory factors, and conformational changes needed for the activity of the polymerase during transcription.

A core promoter (the minimal DNA sequence needed to specify non-regulated or basal transcription) serves to position a Pol in a state termed the Pre-initiation Complex (PIC). In this state, Pol and the GTFs are all bound to the promoter but are not in an active conformation to begin transcription.

Eukaryotic cells contain three Pols, denoted as I, II, and III, which differ in subunit composition.

Genes transcribed by a particular Pol are assigned correspondingly to class I, II, or III.

Pol I transcribes genes for pre-rRNAs. Pol II transcribes all protein-coding genes and genes for snRNAs other than U6 snRNA. Pol III transcribes genes for the 5S rRNA, tRNAs, U6 snRNA, 7SK RNA, 7SL RNA; Alu repeats; some viral genes; and genes for small stable untranslated RNAs.

The genes of the different classes differ in promoter structure, which determines the basal (general) transcription factors and Pol involved in the formation of the PIC.

RNA polymerase II (Pol II) is responsible for the flow of genetic information from DNA to messenger RNA (mRNA) in eukaryotic cells. Studies have identified GTFs—TFIIA, TFIIB, TFIID, TFIIE, TFIIF, and TFIIH—that, together with Pol II, assemble at the promoter site into the PIC and direct transcription initiation at a basal activity level. Further modulation of transcription activity depends on cis control elements in the DNA template that are recognized by sequence-specific activators/repressors assisted by a co-activator.

Sequence elements found in a Pol II core promoters include the TATA element (TATA-binding protein (TBP) binding site), BRE (TFIIB recognition element), Inr (initiator element), and DPE (downstream promoter element). Most promoters contain one or more of these elements, but there is no one element that is absolutely essential for promoter function. The promoter elements are binding sites for subunits of the transcription machinery and serve to orient the transcription machinery at the promoter asymmetrically to direct unidirectional transcription.

The core domain of TBP consists of two imperfect repeats forming a molecule that binds the DNA at the 8-bp TATA element. At TATA-containing promoters, formation of this protein-DNA complex is the initial step in assembly of the transcription machinery. The TATA-like sequence is located about 30 bp upstream of the transcription start site.

RNA polymerase III (Pol III) has the most complex structure among all eukaryotic Pols: the enzyme consists of 17 subunits ranging from ˜10 kDa to −160 kDa and has a total molecular weight of 600-680 kDa.

Class III genes, transcribed by Pol III, comprise three structurally varied promoters, which mostly have an intragenic location. General transcription factors of the Pol III machinery are TFIIIA, TFIIIB, TFIIIC, and the small nuclear RNA-activating protein complex (SNAPc).

The assembly of PICs on different promoters of class III genes (type 1, 2, 3) requires one or more of the A-, B-, and C-boxes; internal control region (ICR); TATA box; distal (DSE) and proximal (PSE) sequence elements. Type 1 genes comprise an A-box at location +57 and a C-box at location +90 relative to the transcription start at +1. Type 2 genes comprise an A-box and a B-box. Type 3 genes comprise a DSE at location −250, a PSE at location −60 and a TATA box at location −27 relative to the transcription start at +1. An A-box may be present, but is not required.

The recruitment and transcription initiation of Pol III on all three types of promoters requires the action of the transcription factor IIIB (TFIIIB) and is highly regulated. The TFIIIB binding site is +/−8 nt around the TATA box. In addition, the TBP is required for transcription by all three polymerases (Han, Y., et al., Cell. Discover. 4 (2018) 40).

With respect to the three types of Pol III genes, Oler, A. J., et al. (Nat. Struct. Mol. Biol. 17 (2010) 620-628) outlined the factors required for directing Pol III to target genes and the three ‘Types’ of Pol III genes in humans based on 1) the presence and positions of cis regulatory elements, and 2) the requirement for particular basal or accessory transcription factors. Briefly, 5S rRNA is the sole Type 1 gene, uniquely requiring TFIIIA. Type 1 and Type 2 genes both require TFIIIC, a basal factor and targeting complex, which recognizes gene-internal A-box and B-box elements at Type 2, but not Type 1 genes. The TFIIIB complex includes the TBP, needed for TATA/promoter recognition and Pol III initiation. Type 2 and 3 genes utilize alternative assemblies of TFIIIB: BRF1 (TFIIIB-related factor 1) for Type 2 and BRF2 (TFIIIB-related factor 2) for Type 3 genes. Type 3 genes lack an internal A- or B-box, and lack reliance on TFIIIC—relying instead on upstream PSE and DSE and specific factors (OCT1, SNAPc, others) for targeting. Notably, Type 3 Pol III promoters resemble Pol II genes in their architecture, which utilizes upstream regulatory elements rather than gene-internal elements.

The novel adenoviral VA RNA gene according to the current invention comprises in certain embodiments in 5′- to 3′-direction in the following order
at least the six 5′-terminal nucleotides of the adenoviral VA RNAI comprising the transcription start site (TSS) (to prevent by-passing of the subsequent polymerase III (poly III) terminator);
a functional polymerase III terminator (to prevent transcription of reverse complementary VA RNA from an optionally present constitutively active upstream promoter),
the adenoviral VA RNAI sequence in inverted form (3′- to 5′-direction).

In certain embodiments of all aspects and embodiments, the VA RNA gene further comprises fused to its 5′-end a polymerase promoter.

In certain embodiments of all aspects and embodiments, the adenoviral VA RNA gene according to the current invention further comprises either directly or via a nucleotide linker fused to its 5′-end a Cre-recombination site of SEQ ID NO: 03. In certain embodiments, the adenoviral VA RNA gene according to the invention comprises fused at its 5′-end either directly or via a nucleotide linker a Cre-recombinase site of SEQ ID NO: 03 and at its 3′-end either directly or via a nucleotide linker a Cre-recombinase site of SEQ ID NO: 06.

In certain embodiments of all aspects and embodiments, the adenoviral VA RNA sequence according to the invention comprises all or a part of the wild-type sequence of SEQ ID NO: 62 or SEQ ID NO: 81 or SEQ ID NO: 83:

gggcactctt ccgtggtctg gtggataaat tcgcaagggt
atcatggcgg acgaccgggg ttcgaacccc ggatccggcc
gtccgccgtg atccatgcgg ttaccgcccg cgtgtcgaac
ccaggtgtgc gacgtcagac aacgggggag cgctcctttt
ggcttccttc caggcgcggc ggctgctgcg ctagcttttt
t.

In certain embodiments of all aspects and embodiments, the adenoviral VA RNA sequence according to the invention comprises all or a part of the wild-type sequence with the mutations G58T, G59T and C68A (sequential numbering) (SEQ ID NO: 62):

gggcactctt ccgtggtctg gtggataaat tcgcaagggt
atcatggcgg acgaccgttg ttcgaacacc ggatccggcc
gtccgccgtg atccatgcgg ttaccgcccg cgtgtcgaac
ccaggtgtgc gacgtcagac aacgggggag cgctcctttt
ggcttccttc caggcgcggc ggctgctgcg ctagcttttt
t.

FIG. 15 shows an alignment comprising the above sequences of SEQ ID NO: 62 and 63.

Said adenoviral VA RNA gene according to the current invention fused to SEQ ID NO: 03 at the 5′-end and to SEQ ID NO: 06 at the 3′-end is shown in FIG. 16 prior to RMCI and in FIG. 17 after RMCI.

In certain embodiments, the adenoviral VA RNA according to the invention comprises the following sequences in 5′- to 3′-direction in the following order:

(1)
(SEQ ID NO: 03)
taccgttcgt ataagtttat atatacgaag ttat
(1a) optionally a stuffer sequence
(SEQ ID NO: 68)
ggacgaaaca cc
(2)
(SEQ ID NO: 64)
gggcac
(3)
(SEQ ID NO: 65)
tttttt
(4)
(SEQ ID NO: 66)
aggagcgctc ccccgttgtc tgacgtcgca cacctgggtt
cgacacgcgg gcggtaaccg catggatcac ggcggacggc
cggatccggt gttcgaacaa cggtcgtccg ccatgatacc
cttgcgaatt tatccaccag accacggaag agtgccc
(5)
(SEQ ID NO 06)
taccgttcgt atatataaac ttatacgaag ttat

In certain embodiments, the adenoviral VA RNA gene according to the invention comprises the sequence of

(SEQ ID NO: 67)
taccgttcgt ataagtttat atatacgaag ttatggacga
aacaccgggc acttttttca gtggccaaaa aagctagcgc
agcagccgcc gcgcctggaa ggaagccaaa aggagcgctc
ccccgttgtc tgacgtcgca cacctgggtt cgacacgcgg
gcggtaaccg catggatcac ggcggacggc cggatccggt
gttcgaacaa cggtcgtccg ccatgatacc cttgcgaatt
tatccaccag accacggaag agtgcccggt gtttcgtcct
accgttcgta tatataaact tatacgaagt tat.

In certain embodiments, the Lx-LE site according to the current invention comprises the following sequence including a stuffer sequence for proper spacing:

(SEQ ID NO: 69)
taccgttcgt ataagtttat atatacgaag ttatggacga
aacacc.

Another aspect of the current invention is a cell comprising the adenoviral VA RNA according to the current invention either in original or inverted form.

Exemplary Uses and Methods Comprising the DNA Element and DNA Molecule According to the Invention

The double stranded DNA element or molecule as well as any nucleic acid according to the invention can be used in the production of recombinant AAV vectors and recombinant AAV particles comprising the same.

Different methods that are known in the art for generating rAAV particles. For example, transfection using AAV plasmid and AAV helper sequences in conjunction with co-infection with one AAV helper virus (e.g., adenovirus, herpesvirus, or vaccinia virus) or transfection with a recombinant AAV plasmid, an AAV helper plasmid, and an helper function plasmid. Non-limiting methods for generating rAAV particles are described, for example, in U.S. Pat. Nos. 6,001,650, 6,004,797, WO 2017/096039, and WO 2018/226887. Following recombinant rAAV particle production (i.e. particle generation in cell culture systems), rAAV particles can be obtained from the host cells and cell culture supernatant and purified.

Aspects of the current invention are methods of transducing cells with a molecule, such as a nucleic acid (e.g., plasmid), according to the invention and production of the respective gene product. Additionally, such cells when transduced with sequences, such as plasmids that encode viral packaging proteins and/or helper proteins can produce recombinant viral particles that include the nucleic acid that encodes a protein of interest or comprises a sequence that is transcribed into a transcript of interest, whereof at least one comprises a DNA element or nucleic acid according to the invention, which in turn produces recombinant viral particles at high yield.

The invention provides viral (e.g., AAV) particle production platform that includes features that distinguish it from current ‘industry-standard’ viral (e.g., AAV) particle production processes by using the nucleic acid or DNA (element) according to the invention.

In discussing nucleic acids (plasmids), a sequence or structure of a particular polynucleotide may be described herein according to the convention of providing the sequence in the 5′ to 3′ direction.

More generally, such cells transfected or transduced with the DNA element or nucleic acid according to the current invention can be referred to as “recombinant cell”. Such a cell can be, for example, a yeast cell, an insect cell, or a mammalian cell, that has been used as recipient of a nucleic acid (plasmid) encoding packaging proteins, such as AAV packaging proteins, a nucleic acid (plasmid) encoding helper proteins, a nucleic acid (plasmid) that encodes a protein or is transcribed into a transcript of interest, i.e. a transgene placed between two AAV ITRs, or other transfer nucleic acid (plasmid), whereof at least one comprises a DNA element or molecule according to the current invention. The term includes the progeny of the original cell, which has been transduced or transfected. It is understood that the progeny of a single parental cell may not necessarily be completely identical in morphology or in genomic or total nucleic acid complement as the original parent, due to natural, accidental, or deliberate mutation.

Numerous cell growth medium appropriate for sustaining cell viability or providing cell growth and/or proliferation are commercially available or can be readily produced. Examples of such medium include serum free eukaryotic growth mediums, such as medium for sustaining viability or providing for the growth of mammalian (e.g., human) cells. Non-limiting examples include Ham's F12 or F12K medium (Sigma-Aldrich), FreeStyle (FS) F17 medium (Thermo-Fisher Scientific), MEM, DMEM, RPMI-1640 (Thermo-Fisher Scientific) and mixtures thereof. Such medium can be supplemented with vitamins and/or trace minerals and/or salts and/or amino acids, such as essential amino acids for mammalian (e.g., human) cells.

Helper protein plasmids can be in the form of a plasmid, phage, transposon or cosmid. In particular, it has been demonstrated that the full-complement of adenovirus genes are not required for helper functions. For example, adenovirus mutants incapable of DNA replication and late gene synthesis have been shown to be permissive for AAV replication. Ito et al., J. Gen. Virol. 9 (1970) 243; Ishibashi et al, Virology 45 (1971) 317.

Mutants within the E2B and E3 regions have been shown to support AAV replication, indicating that the E2B and E3 regions are probably not involved in providing helper function. Carter et al., Virology 126 (1983) 505. However, adenoviruses defective in the E1 region, or having a deleted E4 region, are unable to support AAV replication. Thus, for adenoviral helper proteins, E1A and E4 regions are likely required for AAV replication, either directly or indirectly (see, e.g., Laughlin et al., J. Virol. 41 (1982) 868; Janik et al., Proc. Natl. Acad. Sci. USA 78 (1981) 1925; Carter et al., Virology 126 (1983) 505). Other characterized adenoviral mutants include: E1B (Laughlin et al. (1982), supra; Janik et al. (1981), supra; Ostrove et al., Virology 104 (1980) 502); E2A (Handa et al., J. Gen. Virol. 29 (1975) 239; Strauss et al., J. Virol. 17 (1976) 140; Myers et al., J. Virol. 35 (1980) 665; Jay et al., Proc. Natl. Acad. Sci. USA 78 (1981) 2927; Myers et al., J. Biol. Chem. 256 (1981) 567); E2B (Carter, Adeno-Associated Virus Helper Functions, in I CRC Handbook of Parvoviruses (P. Tijssen ed., 1990)); E3 (Carter et al. (1983), supra); and E4 (Carter et al. (1983), supra; Carter (1995)).

Studies of the helper proteins provided by adenoviruses having mutations in the E1B have reported that the E1B 55 kDa protein is required for AAV particle production, while E1B 19 kDa is not. In addition, WO 97/17458 and Matshushita et al. (Gene Therapy 5 (1998) 938-945) described helper function plasmids encoding various adenoviral genes. An example of a helper plasmid comprise an adenovirus VA RNA coding region, an adenovirus E4 ORF6 coding region, an adenovirus E2A 72 kDa coding region, an adenovirus E1A coding region, and an adenovirus E1B region lacking an intact E1B 55 kDa coding region (see, e.g., WO 01/83797).

Thus, herein is provided a method for producing recombinant AAV vectors or AAV particles comprising said recombinant AAV vectors, which comprise a nucleic acid that encodes a protein or is transcribed into a transcript of interest, using the DNA element or nucleic acid or DNA according to the current invention.

One aspect of the current invention is a method for producing recombinant AAV vectors or AAV particles comprising said recombinant AAV vectors, which comprise a nucleic acid that encodes a protein or is transcribed into a transcript of interest, comprises the steps of
(i) providing one or more plasmids comprising nucleic acids encoding AAV packaging proteins and/or nucleic acids encoding helper proteins, whereof at least one comprises a DNA element or molecule according to the current invention;
(ii) providing a plasmid comprising a nucleic acid that encodes a protein of interest or is transcribed into a transcript of interest;
(iii) contacting one or more mammalian or insect cells with the provided plasmids;
(iv) either further adding a transfection reagent and optionally incubating the plasmid/transfection reagent/cell mixture; or providing a physical means, such as an electric current, to introduce the nucleic acid into the cells;
(v) cultivating the transfected cells and inducing the RMCI at a certain point/cultivation time during the cultivation;
(vi) harvesting the cultivated cells and/or culture medium from the cultivated cells to produce a cell and/or culture medium harvest; and
(vii) isolating and/or purifying recombinant AAV vector or AAV particle from the cell and/or culture medium harvest thereby producing recombinant AAV vector or AAV particle comprising a nucleic acid that encodes a protein of interest or is transcribed into a transcript of interest.

One aspect of the current invention is a method for producing recombinant AAV vectors or AAV particles comprising said recombinant AAV vectors, which comprise a nucleic acid that encodes a protein or is transcribed into a transcript of interest, comprises the steps of
(i) providing one or more plasmids comprising nucleic acids encoding AAV packaging proteins and/or nucleic acids encoding helper proteins, whereof at least one comprises a DNA element or molecule according to the current invention;
(ii) providing a plasmid comprising a nucleic acid that encodes a protein of interest or is transcribed into a transcript of interest;
(iii) contacting one or more mammalian or insect cells with the provided plasmids of (i);
(iv) either further adding a transfection reagent and optionally incubating the plasmid/transfection reagent/cell mixture; or providing a physical means, such as an electric current, to introduce the nucleic acid into the cells;
(v) selecting a stably transfected cell;
(vi) contacting the selected cell of (v) with the provided plasmids of (ii);
(vii) either further adding a transfection reagent and optionally incubating the plasmid/transfection reagent/cell mixture; or providing a physical means, such as an electric current, to introduce the nucleic acid into the cells;
(viii) cultivating the transfected cells of (viii) and inducing the RMCI at a certain point/cultivation time during the cultivation;
(ix) harvesting the cultivated cells and/or culture medium from the cultivated cells to produce a cell and/or culture medium harvest; and
(x) isolating and/or purifying recombinant AAV vector or AAV particle from the cell and/or culture medium harvest thereby producing recombinant AAV vector or AAV particle comprising a nucleic acid that encodes a protein of interest or is transcribed into a transcript of interest.

One aspect of the current invention is a method for producing recombinant AAV vectors or AAV particles comprising said recombinant AAV vectors, which comprise a nucleic acid that encodes a protein or is transcribed into a transcript of interest, comprises the steps of
(i) providing a mammalian or insect cell comprising nucleic acids encoding AAV packaging proteins and/or nucleic acids encoding helper proteins, whereof at least one comprises a DNA element or molecule according to the current invention;
(ii) providing a plasmid comprising a nucleic acid that encodes a protein of interest or is transcribed into a transcript of interest;
(iii) contacting the cell of (i) with the provided plasmid of (ii);
(iv) either further adding a transfection reagent and optionally incubating the plasmid/transfection reagent/cell mixture; or providing a physical means, such as an electric current, to introduce the nucleic acid into the cell;
(v) selecting a stably transfected cell;
(vi) cultivating the stably transfected cell of (v) and inducing the RMCI at a certain point/cultivation time during the cultivation;
(vii) harvesting the cultivated cells and/or culture medium from the cultivated cells to produce a cell and/or culture medium harvest; and
(viii) isolating and/or purifying recombinant AAV vector or AAV particle from the cell and/or culture medium harvest thereby producing recombinant AAV vector or AAV particle comprising a nucleic acid that encodes a protein of interest or is transcribed into a transcript of interest.

The introduction of the nucleic acid comprising the DNA element or molecule according to the current invention into cells can be done in multiple ways.

Diverse methods for the DNA transfer into mammalian cells have been reported in the art. These are all useful in the methods according to the current invention. In certain embodiments of all aspects and embodiments, electroporation, nucleofection, or microinjection for nucleic acid transfer/transfection is used. In certain embodiments of all aspects and embodiments, an inorganic substance (such as, e.g., calcium phosphate/DNA co-precipitation), a cationic polymer (such as, e.g., polyethylenimine, DEAE-dextran), or a cationic lipid (lipofection) is used for nucleic acid transfer/transfection is used. Calcium phosphate and polyethylenimine are the most commonly used reagents for transfection for nucleic acid transfer in larger scales (see, e.g., Baldi et al., Biotechnol. Lett. 29 (2007) 677-684), whereof polyethylenimine is preferred.

In certain embodiments of all aspects and embodiments, the nucleic acid comprising the DNA element or molecule according to the current invention is provided in a composition in combination with polyethylenimine (PEI), optionally in combination with cells. In certain embodiments, the composition includes a plasmid/PEI mixture, which has a plurality of components: (a) one or more plasmids comprising nucleic acids encoding AAV packaging proteins and/or nucleic acids encoding helper proteins whereof at least one comprises a DNA element or molecule according to the invention; (b) a plasmid comprising a nucleic acid that encodes a protein or is transcribed into a transcript of interest; and (c) a polyethylenimine (PEI) solution. In certain embodiments, the plasmids are in a molar ratio range of about 1:0.01 to about 1:100, or are in a molar ratio range of about 100:1 to about 1:0.01, and the mixture of components (a), (b) and (c) has optionally been incubated for a period of time from about 10 seconds to about 4 hours.

In certain embodiments of all aspects and embodiments, the compositions further comprise cells. In certain embodiments, the cells are in contact with the plasmid/PEI mixture of components (a), (b) and/or (c).

In certain embodiments of all aspects and embodiments, the composition, optionally in combination with cells, further comprise free PEI. In certain embodiments, the cells are in contact with the free PEI.

In certain embodiments of all aspects and embodiments, the cells have been in contact with the mixture of components (a), (b) and/or (c) for at least about 4 hours, or about 4 hours to about 140 hours, or for about 4 hours to about 96 hours. In one preferred embodiment, the cells have been in contact with the mixture of components (a), (b) and/or (c) and optionally free PEI, for at least about 4 hours.

Beside a nucleic acid comprising the DNA element or molecule according to the invention the composition may comprise further plasmids. Such plasmids and cells may be in contact with free PEI. In certain embodiments, the plasmids and/or cells have been in contact with the free PEI for at least about 4 hours, or about 4 hours to about 140 hours, or for about 4 hours to about 96 hours.

The invention also provides methods for producing transfected cells using a nucleic acid comprising a DNA element or molecule according to the current invention. The method includes the steps of providing a nucleic acid comprising a DNA element or molecule according to the current invention and optionally one or more additional plasmids; providing a solution comprising polyethylenimine (PEI); and mixing the nucleic acid and optionally the plasmid(s) with the PEI solution to produce a nucleic acid/plasmid/PEI mixture. In certain embodiments, such mixtures are incubated for a period in the range of about 10 seconds to about 4 hours. In such methods, cells are then contacted with the nucleic acid/plasmid/PEI mixture to produce a nucleic acid/plasmid/PEI cell culture; then free PEI is added to the nucleic acid/plasmid/PEI cell culture produced to produce a free PEI/nucleic acid/plasmid/PEI cell culture; and then the free PEI/nucleic acid/plasmid/PEI cell culture produced is incubated for at least about 4 hours, thereby producing transfected cells. In certain embodiments, the plasmid comprises a nucleic acid that encodes a protein or is transcribed into a transcript of interest.

Further provided are methods for producing transfected cells that produce recombinant AAV vector or AAV particle, which include providing one or more plasmids comprising nucleic acids encoding AAV packaging proteins and/or nucleic acids encoding helper proteins, wherein at least one thereof comprises a DNA element or molecule according to the current invention; providing a plasmid comprising a nucleic acid that encodes a protein or is transcribed into a transcript of interest; providing a solution comprising polyethylenimine (PEI); mixing the aforementioned plasmids with the PEI solution, wherein the plasmids are in a molar ratio range of about 1:0.01 to about 1:100, or are in a molar ratio range of about 100:1 to about 1:0.01, to produce a plasmid/PEI mixture (and optionally incubating the plasmid/PEI mixture for a period in the range of about 10 seconds to about 4 hours); contacting cells with the plasmid/PEI mixture, to produce a plasmid/PEI cell culture; adding free PEI to the plasmid/PEI cell culture produced to produce a free PEI/plasmid/PEI cell culture; and incubating the free PEI/plasmid/PEI cell culture for at least about 4 hours, thereby producing transfected cells that produce recombinant AAV vector or particle comprising a nucleic acid that encodes a protein or is transcribed into a transcript of interest.

Additionally provided are methods for producing recombinant AAV vector or AAV particle comprising a nucleic acid that encodes a protein or is transcribed into a transcript of interest, which includes providing one or more plasmids comprising nucleic acids encoding AAV packaging proteins and/or nucleic acids encoding helper proteins whereof at least one comprises a DNA element or molecule according to the current invention; providing a plasmid comprising a nucleic acid that encodes a protein of interest or is transcribed into a transcript of interest; providing a solution comprising polyethylenimine (PEI); mixing the aforementioned plasmids with the PEI solution, wherein the plasmids are in a molar ratio range of about 1:0.01 to about 1:100, or are in a molar ratio range of about 100:1 to about 1:0.01, to produce a plasmid/PEI mixture (and optionally incubating the plasmid/PEI mixture for a period of time in the range of about 10 seconds to about 4 hours); contacting cells with the plasmid/PEI mixture produced as described to produce a plasmid/PEI cell culture; adding free PEI to the plasmid/PEI cell culture produced as described to produce a free PEI/plasmid/PEI cell culture; incubating the plasmid/PEI cell culture or the free PEI/plasmid/PEI cell culture produced for at least about 4 hours to produce transfected cells; harvesting the transfected cells produced and/or culture medium from the transfected cells produced to produce a cell and/or culture medium harvest; and isolating and/or purifying recombinant AAV vector or particle from the cell and/or culture medium harvest produced thereby producing recombinant AAV vector or particle comprising a nucleic acid that encodes a protein or is transcribed into a transcript of interest.

Methods for producing recombinant AAV vectors or AAV particles using the DNA element according to the current invention can include one or more further steps or features. An exemplary step or feature includes, but is not limited to, a step of harvesting the cultivated cells produced and/or harvesting the culture medium from the cultivated cells produced to produce a cell and/or culture medium harvest. An additional exemplary step or feature includes, but is not limited to isolating and/or purifying recombinant AAV vector or AAV particle from the cell and/or culture medium harvest thereby producing recombinant AAV vector or AAV particle comprising a nucleic acid that encodes a protein or is transcribed into a transcript of interest.

In certain embodiments of all aspects and embodiments, PEI is added to the plasmids and/or cells at various time points. In certain embodiments, free PEI is added the cells before, at the same time as, or after the plasmid/PEI mixture is contacted with the cells.

In certain embodiments of all aspects and embodiments, the cells are at particular densities and/or cell growth phases and/or viability when contacted with the plasmid/PEI mixture and/or when contacted with the free PEI. In one preferred embodiment, cells are at a density in the range of about 1×10E5 cells/mL to about 1×10E8 cells/mL when contacted with the plasmid/PEI mixture and/or when contacted with the free PEI. In certain embodiments, viability of the cells when contacted with the plasmid/PEI mixture or with the free PEI is about 60% or greater than 60%, or wherein the cells are in log phase growth when contacted with the plasmid/PEI mixture, or viability of the cells when contacted with the plasmid/PEI mixture or with the free PEI is about 90% or greater than 90%, or wherein the cells are in log phase growth when contacted with the plasmid/PEI mixture or with the free PEI.

Encoded AAV packaging proteins include, in certain embodiments of all aspects and embodiments, AAV rep and/or AAV cap. Such AAV packaging proteins include, in certain embodiments of all aspects and embodiments, AAV rep and/or AAV cap proteins of any AAV serotype.

Encoded helper proteins include, in certain embodiments of all aspects and embodiments, adenovirus E2 and/or E4, VARNA proteins, and/or non-AAV helper proteins.

In certain embodiments of all aspects and embodiments, the nucleic acids (plasmids) are used at particular amounts or ratios. In certain embodiments, the total amount of plasmid comprising the nucleic acid that encodes a protein or is transcribed into a transcript of interest and the one or more plasmids comprising nucleic acids encoding AAV packaging proteins and/or nucleic acids encoding helper proteins, whereof at least one comprises a DNA element or molecule according to the current invention, is in the range of about 0.1 μg to about 15 μg per mL of cells. In certain embodiments, the molar ratio of the plasmid comprising the nucleic acid that encodes a protein or is transcribed into a transcript of interest to the one or more plasmids comprising nucleic acids encoding AAV packaging proteins and/or nucleic acids encoding helper proteins, whereof at least one comprises a DNA element or molecule according to the invention, is in the range of about 1:5 to about 1:1, or is in the range of about 1:1 to about 5:1.

Plasmids can include nucleic acids on different or the same plasmids. In certain embodiments of all aspects and embodiments, a first plasmid comprises the nucleic acids encoding AAV packaging proteins and a second plasmid comprises the nucleic acids encoding helper proteins. At least one of these nucleic acids comprises a DNA element or molecule according to the current invention.

In certain embodiments of all aspects and embodiments, the molar ratio of the plasmid comprising the nucleic acid that encodes a protein or is transcribed into a transcript of interest to a first plasmid comprising the nucleic acids encoding AAV packaging proteins to a second plasmid comprising the nucleic acids encoding helper proteins is in the range of about 1-5:1:1, or 1: 1-5:1, or 1:1:1-5 in co-transfection.

In certain embodiments of all aspects and embodiments, the cell is a eukaryotic cell. In certain embodiments, the eukaryotic cell is a mammalian cell. In one preferred embodiment, the cell is a HEK293 cell or a CHO cell.

The cultivation can be performed using the generally used conditions for the cultivation of eukaryotic cells of about 37° C., 95% humidity and 8 vol.-% CO₂. The cultivation can be performed in serum containing or serum free medium, in adherent culture or in suspension culture. The suspension cultivation can be performed in any fermentation vessel, such as, e.g., in stirred tank reactors, wave reactors, in shaker vessels or spinner vessels or in so called roller bottles. Transfection can be performed in high throughput format and screening, respectively, e.g. in a 96 or 384 well format.

Methods according to the current invention include AAV particles of any serotype, or a variant thereof. In certain embodiments of all aspects and embodiments, a recombinant AAV particle comprises any of AAV serotypes 1-12, an AAV VP1, VP2 and/or VP3 capsid protein, or a modified or variant AAV VP1, VP2 and/or VP3 capsid protein, or wild-type AAV VP1, VP2 and/or VP3 capsid protein. In certain embodiments of all aspects and embodiments, an AAV particle comprises an AAV serotype or an AAV pseudotype, where the AAV pseudotype comprises an AAV capsid serotype different from an ITR serotype.

Methods according to the invention that provide or include AAV vectors or particles can also include other elements. Examples of such elements include but are not limited to: an intron, an expression control element, one or more adeno-associated virus (AAV) inverted terminal repeats (ITRs) and/or a filler/stuffer polynucleotide sequence. Such elements can be within or flank the nucleic acid that encodes a protein or is transcribed into a transcript of interest, or the expression control element can be operably linked to nucleic acid that encodes a protein or is transcribed into a transcript of interest, or the AAV ITR(s) can flank the 5′- or 3′-terminus of nucleic acid that encodes a protein or is transcribed into a transcript of interest, or the filler polynucleotide sequence can flank the 5′- or 3′-terminus of nucleic acid that encodes a protein or is transcribed into a transcript of interest.

Expression control elements include constitutive or regulatable control elements, such as a tissue-specific expression control element or promoter (e.g. that provides for expression in liver).

ITRs can be any of: AAV2 or AAV6 or AAV8 or AAVS serotypes, or a combination thereof. AAV particles can include any VP1, VP2 and/or VP3 capsid protein having 75% or more sequence identity to any of AAV1, AAV2, AAV3, AAV4, AAVS, AAV6, AAV10, AAV11, AAV-2i8 or AAV rh74 VP1, VP2 and/or VP3 capsid proteins, or comprises a modified or variant VP1, VP2 and/or VP3 capsid protein selected from any of: AAV1, AAV2, AAV3, AAV4, AAV5, AAV6, AAV10, AAV11, AAV-2i8 and AAV rh74 AAV serotypes.

Following production of recombinant viral (e.g., AAV) particles as set forth herein, if desired, the viral (e.g., rAAV) particles can be purified and/or isolated from host cells using a variety of conventional methods. Such methods include column chromatography, CsCl gradients, and the like. For example, a plurality of column purification steps such as purification over an anion exchange column, an affinity column and/or a cation exchange column can be used. (See, e.g., WO 02/12455 and US 2003/0207439). Alternatively, or in addition, CsCl gradient steps can be used (see, e.g., US 2012/0135515; and US 2013/0072548). Further, if the use of infectious virus is employed to express the packaging and/or helper proteins, residual virus can be inactivated, using various methods. For example, adenovirus can be inactivated by heating to temperatures of approximately 60° C. for, e.g., 20 minutes or more. This treatment effectively inactivates the helper virus since AAV is heat stable while the helper adenovirus is heat labile.

Viral vectors such as parvo-virus particles, including AAV serotypes and variants thereof, provide a means for delivery of nucleic acid into cells ex vivo, in vitro and in vivo, which encode proteins such that the cells express the encoded protein. AAVs are viruses useful as gene therapy vectors as they can penetrate cells and introduce nucleic acid/genetic material so that the nucleic acid/genetic material may be stably maintained in cells. In addition, these viruses can introduce nucleic acid/genetic material into specific sites, for example. Because AAV are not associated with pathogenic disease in humans, AAV vectors are able to deliver heterologous polynucleotide sequences (e.g., therapeutic proteins and agents) to human patients without causing substantial AAV pathogenesis or disease.

Viral vectors, which may be used, include, but are not limited to, adeno-associated virus (AAV) particles of multiple serotypes (e.g., AAV-1 to AAV-12, and others) and hybrid/chimeric AAV particles.

AAV particles may be used to advantage as vehicles for effective gene delivery. Such particles possess a number of desirable features for such applications, including tropism for dividing and non-dividing cells. Early clinical experience with these vectors also demonstrated no sustained toxicity and immune responses were minimal or undetectable. AAV are known to infect a wide variety of cell types in vivo and in vitro by receptor-mediated endocytosis or by transcytosis. These vector systems have been tested in humans targeting retinal epithelium, liver, skeletal muscle, airways, brain, joints and hematopoietic stem cells.

Recombinant AAV particles do not typically include viral genes associated with pathogenesis. Such vectors typically have one or more of the wild-type AAV genes deleted in whole or in part, for example, rep and/or cap genes, but retain at least one functional flanking ITR sequence, as necessary for the rescue, replication, and packaging of the recombinant vector into an AAV particle. For example, only the essential parts of the vector e.g., the ITR and LTR elements, respectively, are included. An AAV vector genome would therefore include sequences required in cis for replication and packaging (e.g., functional ITR sequences).

Recombinant AAV vectors, as well as methods and uses thereof, include any viral strain or serotype. As a non-limiting example, a recombinant AAV vector can be based upon any AAV genome, such as AAV-1, -2, -3, -4, -5, -6, -7, -8, -9, -10, -11, -12, 2i8, or AAV rh74 for example. Such vectors can be based on the same strain or serotype (or subgroup or variant), or be different from each other. As a non-limiting example, a recombinant AAV vector based upon one serotype genome can be identical to one or more of the capsid proteins that package the vector. In addition, a recombinant AAV vector genome can be based upon an AAV (e.g., AAV2) serotype genome distinct from one or more of the AAV capsid proteins that package the vector. For example, the AAV vector genome can be based upon AAV2, whereas at least one of the three capsid proteins could be an AAV1, AAV3, AAV4, AAVS, AAV6, AAV7, AAV8, AAV9, AAV10, AAV11, AAV12, AAV-2i8, or AAV rh74 or variant thereof, for example. AAV variants include variants and chimeras of AAV1, AAV2, AAV3, AAV4, AAV5, AAV6, AAV7, AAV8, AAV9, AAV10, AAV11, AAV12, AAV-2i8 and AAV rh74 capsids.

In certain embodiments of all aspects and embodiments, adeno-associated virus (AAV) vectors include AAV1, AAV2, AAV3, AAV4, AAV5, AAV6, AAV7, AAV8, AAV9, AAV10, AAV11, AAV12, AAV-2i8, and AAV rh74, as well as variants (e.g., capsid variants, such as amino acid insertions, additions, substitutions and deletions) thereof, for example, as set forth in WO 2013/158879, WO 2015/013313 and US 2013/0059732 (disclosing LK01, LK02, LK03, etc.).

AAV and AAV variants (e.g., capsid variants) serotypes (e.g., VP1, VP2, and/or VP3 sequences) may or may not be distinct from other AAV serotypes, including, for example, AAV1-AAV12 (e.g., distinct from VP1, VP2, and/or VP3 sequences of any of AAV1-AAV12 serotypes).

In certain embodiments of all aspects and embodiments, an AAV particle related to a reference serotype has a polynucleotide, polypeptide or subsequence thereof that includes or consists of a sequence at least 80% or more (e.g., 85%, 90%, 95%, 96%, 97%, 98%, 99%, 99.1%, 99.2%, 99.3%, 99.4%, 99.5%, etc.) identical to one or more AAV1, AAV2, AAV3, AAV4, AAV5, AAV6, AAV7, AAV8, AAV9, AAV10, AAV11, AAV12, AAV-2i8 or AAV rh74 (e.g., such as an ITR, or a VP1, VP2, and/or VP3 sequences).

Compositions, methods and uses of the invention include AAV sequences (polypeptides and nucleotides), and subsequences thereof that exhibit less than 100% sequence identity to a reference AAV serotype such as AAV1, AAV2, AAV3, AAV4, AAV5, AAV6, AAV7, AAV8, AAV9, AAV10, AAV11, AAV12, AAV-2i8, or AAV rh74, but are distinct from and not identical to known AAV genes or proteins, such as AAV1, AAV2, AAV3, AAV4, AAV5, AAV6, AAV7, AAV8, AAV9, AAV10, AAV11, AAV12, AAV-2i8, or AAV rh74, genes or proteins, etc. In certain embodiments of all aspects and embodiments, an AAV polypeptide or subsequence thereof includes or consists of a sequence at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to any reference AAV sequence or subsequence thereof, such as AAV1, AAV2, AAV3, AAV4, AAV5, AAV6, AAV7, AAV8, AAV9, AAV10, AAV11, AAV12, AAV-2i8, or AAV rh74 (e.g., VP1, VP2 and/or VP3 capsid or ITR). In certain embodiments, an AAV variant has 1, 2, 3, 4, 5, 5-10, 10-15, 15-20 or more amino acid substitutions.

Recombinant AAV particles, including AAV1, AAV2, AAV3, AAV4, AAVS, AAV6, AAV7, AAV8, AAV9, AAV10, AAV11, AAV12, AAV-2i8, or AAV rh74, and variant, related, hybrid and chimeric sequences, can be constructed using recombinant techniques that are known to the skilled artisan, to include one or more nucleic acid sequences (transgenes) flanked with one or more functional AAV ITR sequences.

Recombinant particles (e.g., rAAV particles) can be incorporated into pharmaceutical compositions. Such pharmaceutical compositions are useful for, among other things, administration and delivery to a subject in vivo or ex vivo. In certain embodiments, pharmaceutical compositions contains a pharmaceutically acceptable carrier or excipient. Such excipients include any pharmaceutical agent that does not itself induce an immune response harmful to the individual receiving the composition, and which may be administered without undue toxicity.

Protocols for the generation of adenoviral vectors have been described in U.S. Pat. Nos. 5,998,205; 6,228,646; 6,093,699; 6,100,242; WO 94/17810 and WO 94/23744, which are incorporated herein by reference in their entirety.

Despite the pathogenicity for humans, an objective in the rAAV vector production and purification systems is to implement strategies to minimize/control the generation of production related impurities such as proteins, nucleic acids, and vector-related impurities, including wild-type/pseudo wild-type AAV species (wtAAV) and AAV-encapsulated residual DNA impurities.

Considering that the rAAV particle represents only a minor fraction of the biomass, rAAV particles need to be purified to a level of purity, which can be used as a clinical human gene therapy product (see, e.g., Smith P. H., et al., Mo. Therapy 7 (2003) 8348; Chadeuf G., et al, Mo. Therapy 12 (2005) 744; report from the CHMP gene therapy expert group meeting, European Medicines Agency EMEA/CHMP 2005, 183989/2004).

As an initial step, typically the cultivated cells that produce the rAAV particles are harvested, optionally in combination with harvesting cell culture supernatant (medium) in which the cells (suspension or adherent) producing rAAV particles have been cultured. The harvested cells and optionally cell culture supernatant may be used as is, as appropriate, or concentrated. Further, if infection is employed to express helper functions, residual helper virus can be inactivated. For example, adenovirus can be inactivated by heating to temperatures of approximately 60° C. for, e.g., 20 minutes or more, which inactivates only the helper virus since AAV is heat stable while the helper adenovirus is heat labile.

Cells and/or supernatant of the harvest are lysed by disrupting the cells, for example, by chemical or physical means, such as detergent, microfluidization and/or homogenization, to release the rAAV particles. Concurrently during cell lysis or subsequently after cell lysis, a nuclease, such as, e.g., benzonase, is added to degrade contaminating DNA. Typically, the resulting lysate is clarified to remove cell debris, e.g. by filtering or centrifuging, to render a clarified cell lysate. In a particular example, lysate is filtered with a micron diameter pore size filter (such as a 0.1-10.0 μm pore size filter, for example, a 0.45 μm and/or pore size 0.2 μm filter), to produce a clarified lysate.

The lysate (optionally clarified) contains AAV particles (comprising rAAV vectors as well as empty capsids) and production/process related impurities, such as soluble cellular components from the host cells that can include, inter alia, cellular proteins, lipids, and/or nucleic acids, and cell culture medium components. The optionally clarified lysate is then subjected to purification steps to purify AAV particles (comprising rAAV vectors) from impurities using chromatography. The clarified lysate may be diluted or concentrated with an appropriate buffer prior to the first chromatography step.

After cell lysis, optional clarifying, and optional dilution or concentration, a plurality of subsequent and sequential chromatography steps can be used to purify rAAV particles.

A first chromatography step may be cation exchange chromatography or anion exchange chromatography. If the first chromatography step is cation exchange chromatography the second chromatography step can be anion exchange chromatography or size exclusion chromatography (SEC). Thus, in certain embodiments of all aspects and embodiments, rAAV particle purification is via cation exchange chromatography, followed by purification via anion exchange chromatography.

Alternatively, if the first chromatography step is cation exchange chromatography the second chromatography step can be size exclusion chromatography (SEC). Thus, in certain embodiments of all aspects and embodiments, rAAV particle purification is via cation exchange chromatography, followed by purification via size exclusion chromatography (SEC).

Still alternatively, a first chromatography step may be affinity chromatography. If the first chromatography step is affinity chromatography the second chromatography step can be anion exchange chromatography. Thus, in certain embodiments of all aspects and embodiments, rAAV particle purification is via affinity chromatography, followed by purification via anion exchange chromatography.

Optionally, a third chromatography can be added to the foregoing chromatography steps. Typically, the optional third chromatography step follows cation exchange, anion exchange, size exclusion or affinity chromatography.

Thus, in certain embodiments of all aspects and embodiments, rAAV particle purification is via cation exchange chromatography, followed by purification via anion exchange chromatography, followed by purification via size exclusion chromatography (SEC).

In addition, in certain embodiments of all aspects and embodiments, further rAAV particle purification is via cation exchange chromatography, followed by purification via size exclusion chromatography (SEC), followed by purification via anion exchange chromatography.

In yet further embodiments of all aspects and embodiments, rAAV particle purification is via affinity chromatography, followed by purification via anion exchange chromatography, followed by purification via size exclusion chromatography (SEC).

In yet further embodiments of all aspects and embodiments, rAAV particle purification is via affinity chromatography, followed by purification via size exclusion chromatography (SEC), followed by purification via anion exchange chromatography.

Cation exchange chromatography functions to separate the AAV particles from cellular and other components present in the clarified lysate and/or column eluate from an affinity or size exclusion chromatography. Examples of strong cation exchange resins capable of binding rAAV particles over a wide pH range include, without limitation, any sulfonic acid based resin as indicated by the presence of the sulfonate functional group, including aryl and alkyl substituted sulfonates, such as sulfopropyl or sulfoethyl resins. Representative matrices include but are not limited to POROS HS, POROS HS 50, POROS XS, POROS SP, and POROS S (strong cation exchangers available from Thermo Fisher Scientific, Inc., Waltham, Mass., USA). Additional examples include Capto S, Capto S ImpAct, Capto S ImpRes (strong cation exchangers available from GE Healthcare, Marlborough, Mass., USA), and commercial DOWEX®, AMBERLITE®, and AMBERLYST® families of resins available from Aldrich Chemical Company (Milliwaukee, Wis., USA). Weak cation exchange resins include, without limitation, any carboxylic acid based resin. Exemplary cation exchange resins include carboxymethyl (CM), phospho (based on the phosphate functional group), methyl sulfonate (S) and sulfopropyl (SP) resins.

Anion exchange chromatography functions to separate AAV particles from proteins, cellular and other components present in the clarified lysate and/or column eluate from an affinity or cation exchange or size exclusion chromatography. Anion exchange chromatography can also be used to reduce and thereby control the amount of empty capsids in the eluate. For example, the anion exchange column having rAAV particle bound thereto can be washed with a solution comprising NaCl at a modest concentration (e.g., about 100-125 mM, such as 110-115 mM) and a portion of the empty capsids can be eluted in the flow through without substantial elution of the rAAV particles. Subsequently, rAAV particles bound to the anion exchange column can be eluted using a solution comprising NaCl at a higher concentration (e.g., about 130-300 mM NaCl), thereby producing a column eluate with reduced or depleted amounts of empty capsids and proportionally increased amounts of rAAV particles comprising an rAAV vector.

Exemplary anion exchange resins include, without limitation, those based on polyamine resins and other resins. Examples of strong anion exchange resins include those based generally on the quaternized nitrogen atom including, without limitation, quaternary ammonium salt resins such as trialkylbenzyl ammonium resins. Suitable exchange chromatography materials include, without limitation, MACRO PREP Q (strong anion-exchanger available from BioRad, Hercules, Calif., USA); UNOSPHERE Q (strong anion-exchanger available from BioRad, Hercules, Calif., USA); POROS 50HQ (strong anion-exchanger available from Applied Biosystems, Foster City, Calif., USA); POROS XQ (strong anion-exchanger available from Applied Biosystems, Foster City, Calif., USA); POROS SOD (weak anion-exchanger available from Applied Biosystems, Foster City, Calif., USA); POROS 50P1 (weak anion-exchanger available from Applied Biosystems, Foster City, Calif., USA); Capto Q, Capto XQ, Capto Q ImpRes, and SOURCE 30Q (strong anion-exchanger available from GE healthcare, Marlborough, Mass., USA); DEAE SEPHAROSE (weak anion-exchanger available from Amersham Biosciences, Piscataway, N.J., USA); Q SEPHAROSE (strong anion-exchanger available from Amersham Biosciences, Piscataway, N.J., USA). Additional exemplary anion exchange resins include aminoethyl (AE), diethylaminoethyl (DEAE), diethylaminopropyl (DEPE) and quaternary amino ethyl (QAE).

A manufacturing process to purify recombinant AAV particles intended as a product to treat human disease should achieve the following objectives: 1) consistent particle purity, potency and safety; 2) manufacturing process scalability; and 3) acceptable cost of manufacturing.

Exemplary processes for recombinant AAV particle purification are reported in WO 2019/006390.

The below outlined recombinant adeno-associated virus particle (rAAV particle) purification and production methods are scalable up to large scale. For example, to a suspension culture of 5, 10, 10-20, 20-50, 50-100, 100-200 or more liters volume. The recombinant adeno-associated virus particle purification and production methods are applicable to a wide variety of AAV serotypes/capsid variants.

In certain embodiments of all aspects and embodiments, the purification of rAAV particles comprises the steps of:
(a) harvesting cells and/or cell culture supernatant comprising rAAV particles to produce a harvest;
(b) optionally concentrating the harvest produced in step (a) to produce a concentrated harvest;
(c) lysing the harvest produced in step (a) or the concentrated harvest produced in step (b) to produce a lysate;
(d) treating the lysate produced in step (c) to reduce contaminating nucleic acid in the lysate thereby producing a nucleic acid reduced lysate;
(e) optionally filtering the nucleic acid reduced lysate produced in step (d) to produce a clarified lysate, and optionally diluting the clarified lysate to produce a diluted clarified lysate;
(f) subjecting the nucleic acid reduced lysate of step (d), the clarified lysate of step (e), or the diluted clarified lysate produced in step (e) to a cation exchange column chromatography to produce a column eluate comprising rAAV particles, thereby separating rAAV particles from protein impurities or other production/process related impurities, and optionally diluting the column eluate to produce a diluted column eluate;
(g) subjecting the column eluate or the diluted column eluate produced in step (f) to an anion exchange chromatography to produce a second column eluate comprising rAAV particles, thereby separating rAAV particles from protein impurities or production/process related impurities, and optionally concentrating the second column eluate to produce a concentrated second column eluate;
(h) subjecting the second column eluate or the concentrated second column eluate produced in step (g) to a size exclusion column chromatography (SEC) to produce a third column eluate comprising rAAV particles, thereby separating rAAV particles from protein impurities or production/process related impurities, and optionally concentrating the third column eluate to produce a concentrated third column eluate; and
(i) filtering the third column eluate or the concentrated third column eluate produced in step (h), thereby producing purified rAAV particles.

In certain embodiments, steps (a) to (f) are maintained and combined with the following steps:
(g) subjecting the column eluate or the concentrated column eluate produced in step (f) to a size exclusion column chromatography (SEC) to produce a second column eluate comprising rAAV particles, thereby separating rAAV particles from protein impurities or other production/process related impurities, and optionally diluting the second column eluate to produce a concentrated second column eluate;
(h) subjecting the second column eluate or the diluted second column eluate produced in step (g) to an anion exchange chromatography to produce a third column eluate comprising rAAV particles thereby separating rAAV particles from protein impurities production/process related impurities and optionally diluting the third column eluate to produce a diluted third column eluate; and
(i) filtering the third column eluate or the concentrated third column eluate produced in step (h), thereby producing purified rAAV particles.

In certain embodiments, steps (a) to (g) are maintained and combined with the following step:
(h) filtering the second column eluate or the concentrated second column eluate produced in step (g), thereby producing purified rAAV particles.

In embodiment, steps (a) to (e) are maintained and combined with the following steps:
(f) subjecting the nucleic acid reduced lysate in step (d), or clarified lysate or diluted clarified lysate produced in step (e) to an AAV affinity column chromatography to produce a column eluate comprising rAAV particles, thereby separating rAAV particles from protein impurities or other production/process related impurities, and optionally concentrating the column eluate to produce a concentrated column eluate;
(g) subjecting the column eluate or the concentrated column eluate produced in step (f) to a size exclusion column chromatography (SEC) to produce a second column eluate comprising rAAV particles, thereby separating rAAV particles from protein impurities or other production/process related impurities, and optionally diluting the second column eluate to produce a diluted second column eluate;
(h) optionally subjecting the second column eluate or the diluted second column eluate produced in step (g) to an anion exchange chromatography to produce a third column eluate comprising rAAV particles, thereby separating rAAV particles from protein impurities or other production/process related impurities, and optionally diluting the third column eluate to produce a diluted third column eluate; and
(i) filtering the second column eluate or the diluted second column eluate produced in step (g), or filtering the third column eluate or the concentrated third column eluate produced in step (h), thereby producing purified rAAV particles.

In certain embodiments of all aspects and embodiments, concentrating of step (b) and/or step (f) and/or step (g) and/or step (h) is via ultrafiltration/diafiltration, such as by tangential flow filtration (TFF).

In certain embodiments of all aspects and embodiments, concentrating of step (b) reduces the volume of the harvested cells and cell culture supernatant by about 2-20 fold.

In certain embodiments of all aspects and embodiments, concentrating of step (0 and/or step (g) and/or step (h) reduces the volume of the column eluate by about 5-20 fold.

In certain embodiments of all aspects and embodiments, lysing of the harvest produced in step (a) or the concentrated harvest produced in step (b) is by physical or chemical means. Non-limiting examples of physical means include microfluidization and homogenization. Non-limiting examples of chemical means include detergents. Detergents include non-ionic and ionic detergents. Non-limiting examples of non-ionic detergents include Triton X-100. Non-limiting examples of detergent concentration is between about 0.1 and 1.0% (v/v) or (w/v), inclusive.

In certain embodiments of all aspects and embodiments, step (d) comprises treating with a nuclease thereby reducing contaminating nucleic acid. Non-limiting examples of a nuclease include benzonase.

In certain embodiments of all aspects and embodiments, filtering of the clarified lysate or the diluted clarified lysate of step (e) is via a filter. Non-limiting examples of filters are those having a pore diameter of between about 0.1 and 10.0 microns, inclusive.

In certain embodiments of all aspects and embodiments, diluting of the clarified lysate of step (e) is with an aqueous buffered phosphate, acetate or Tris solution. Non-limiting examples of solution pH are between about pH 4.0 and pH 7.4, inclusive. Non-limiting examples of Tris solution pH are greater than pH 7.5, such as between about pH 8.0 and pH 9.0, inclusive.

In certain embodiments of all aspects and embodiments, diluting of the column eluate of step (f) or the second column eluate of step (g) is with an aqueous buffered phosphate, acetate or Tris solution. Non-limiting examples of solution pH are between about pH 4.0 and pH 7.4, inclusive. Non-limiting examples of Tris solution pH are greater than pH 7.5, such as between about pH 8.0 and pH 9.0, inclusive.

In certain embodiments of all aspects and embodiments, the rAAV particles resulting from step (i) are formulated with a surfactant to produce a rAAV particle formulation.

In certain embodiments of all aspects and embodiments, the anion exchange column chromatography of step (f), (g) and/or (h) comprises polyethylene glycol (PEG) modulated column chromatography.

In certain embodiments of all aspects and embodiments, the anion exchange column chromatography of step (g) and/or (h) is washed with a PEG solution prior to elution of the rAAV particles from the column.

In certain embodiments of all aspects and embodiments, the PEG has an average molecular weight in a range of about 1,000 g/mol to 80,000 g/mol, inclusive.

In certain embodiments of all aspects and embodiments, the PEG is at a concentration of about 4% to about 10% (w/v), inclusive.

In certain embodiments of all aspects and embodiments, the anion exchange column of step (g) and/or (h) is washed with an aqueous surfactant solution prior to elution of the rAAV particles from the column.

In certain embodiments of all aspects and embodiments, the cation exchange column of step (f) is washed with a surfactant solution prior to elution of the rAAV particles from the column.

In certain embodiments of all aspects and embodiments, the PEG solution and/or the surfactant solution comprises an aqueous Tris-HCl/NaCl buffer, an aqueous phosphate/NaCl buffer, or an aqueous acetate/NaCl buffer.

In certain embodiments of all aspects and embodiments, NaCl concentration in the buffer or solution is in a range of between about 20-300 mM NaCl, inclusive, or between about 50-250 mM NaCl, inclusive.

In certain embodiments of all aspects and embodiments, the surfactant comprises a cationic or anionic surfactant.

In certain embodiments of all aspects and embodiments, the surfactant comprises a twelve carbon chained surfactant.

In certain embodiments of all aspects and embodiments, the surfactant comprises Dodecyltrimethylammonium chloride (DTAC) or Sarkosyl.

In certain embodiments of all aspects and embodiments, the rAAV particles are eluted from the anion exchange column of step (f), (g) and/or (h) with an aqueous Tris-HCl/NaCl buffer.

In certain embodiments of all aspects and embodiments, the Tris-HCl/NaCl buffer comprises 100-400 mM NaCl, inclusive, optionally at a pH in a range of about pH 7.5 to about pH 9.0, inclusive.

In certain embodiments of all aspects and embodiments, the anion exchange column of step (f), (g) and/or (h) is washed with an aqueous Tris-HCl/NaCl buffer.

In certain embodiments of all aspects and embodiments, the NaCl concentration in the aqueous Tris-HCl/NaCl buffer is in a range of about 75-125 mM, inclusive.

In certain embodiments of all aspects and embodiments, the aqueous Tris-HCl/NaCl buffer has a pH from about pH 7.5 to about pH 9.0, inclusive.

In certain embodiments of all aspects and embodiments, the anion exchange column of step (f), (g) and/or (h) is washed one or more times to reduce the amount of empty capsids in the second or third column eluate.

In certain embodiments of all aspects and embodiments, the anion exchange column wash removes empty capsids from the column prior to rAAV particle elution and/or instead of rAAV particle elution, thereby reducing the amount of empty capsids in the second or third column eluate.

In certain embodiments of all aspects and embodiments, the anion exchange column wash removes at least about 50% of the total empty capsids from the column prior to rAAV particle elution and/or instead of rAAV particle elution, thereby reducing the amount of empty capsids in the second or third column eluate by about 50%.

In certain embodiments of all aspects and embodiments, the NaCl concentration in the aqueous Tris-HCl/NaCl buffer is in a range of about 110-120 mM, inclusive.

In certain embodiments of all aspects and embodiments, ratios and/or amounts of the rAAV particles and empty capsids eluted are controlled by a wash buffer.

In certain embodiments of all aspects and embodiments, the rAAV particles are eluted from the cation exchange column of step (f) in an aqueous phosphate/NaCl buffer, or an aqueous acetate/NaCl buffer. Non-limiting NaCl concentration in a buffer is in a range of about 125-500 mM NaCl, inclusive. Non-limiting examples of buffer pH are between about pH 5.5 to about pH 7.5, inclusive.

In certain embodiments of all aspects and embodiments, the anion exchange column of step (f), (g) and/or (h) comprises a quaternary ammonium functional group such as quaternized polyethylenimine.

In certain embodiments of all aspects and embodiments, the size exclusion column (SEC) of step (g) and/or (h) has a separation/fractionation range (molecular weight) from about 10,000 g/mol to about 600,000 g/mol, inclusive.

In certain embodiments of all aspects and embodiments, the cation exchange column of step (f) comprises a sulfonic acid or functional group such as sulphopropyl.

In certain embodiments of all aspects and embodiments, the AAV affinity column comprises a protein or ligand that binds to AAV capsid protein. Non-limiting examples of a protein include an antibody that binds to AAV capsid protein. More specific non-limiting examples include a single-chain Llama antibody (Camelid) that binds to AAV capsid protein.

In certain embodiments of all aspects and embodiments, the method excludes a step of cesium chloride gradient ultracentrifugation.

In certain embodiments of all aspects and embodiments, the method recovers approximately 50-90% of the total rAAV particles from the harvest produced in step (a) or the concentrated harvest produced in step (b).

In certain embodiments of all aspects and embodiments, the method produces rAAV particles having a greater purity than rAAV particles produced or purified by a single AAV affinity column purification.

In certain embodiments of all aspects and embodiments, steps (c) and (d) are performed substantially concurrently.

In certain embodiments of all aspects and embodiments, the NaCl concentration is adjusted to be in a range of about 100-400 mM NaCl, inclusive, or in a range of about 140-300 mM NaCl, inclusive, after step (c) but prior to step (f).

In certain embodiments of all aspects and embodiments, the rAAV particles are derived from an AAV selected from the group consisting of AAV1, AAV2, AAV3, AAV4, AAV5, AAV6, AAV7, AAV8, AAV9, AAV10, Rh10 and Rh74.

In certain embodiments of all aspects and embodiments, the rAAV particles comprise a capsid sequence having 70% or more sequence identity to an AAV1, AAV2, AAV3, AAV4, AAV5, AAV6, AAV7, AAV8, AAV9, AAV10, Rh10, Rh74, SEQ ID NO: 75, or SEQ ID NO: 76 capsid sequence.

In certain embodiments of all aspects and embodiments, the rAAV particles comprise an ITR sequence having 70% or more sequence identity to an AAV1, AAV2, AAV3, AAV4, AAV5, AAV6, AAV7, AAV8, AAV9, AAV10, Rh10, or Rh74 ITR sequence.

In certain embodiments of all aspects and embodiments, the cells are suspension growing or adherent growing cells.

In certain embodiments of all aspects and embodiments, the cells are mammalian cells. Non-limiting examples include HEK cells, such as HEK-293 cells, and CHO cells, such as CHO-K1 cells.

Methods to determine infectious titer of rAAV particles containing a transgene are known in the art (see, e.g., Zhen et al., Hum. Gene Ther. 15 (2004) 709). Methods for assaying for empty capsids and rAAV particles with packaged transgenes are known (see, e.g., Grimm et al., Gene Therapy 6 (1999) 1322-1330; Sommer et al., Malec. Ther. 7 (2003) 122-128).

To determine the presence or amount of degraded/denatured capsid, purified rAAV particle can be subjected to SDS-polyacrylamide gel electrophoresis, consisting of any gel capable of separating the three capsid proteins, for example, a gradient gel, then running the gel until sample is separated, and blotting the gel onto nylon or nitrocellulose membranes. Anti-AAV capsid antibodies are then used as primary antibodies that bind to denatured capsid proteins (see, e.g., Wobus et al., J. Viral. 74 (2000) 9281-9293). A secondary antibody that binds to the primary antibody contains a means for detecting the primary antibody. Binding between the primary and secondary antibodies is detected semi-quantitatively to determine the amount of capsids. Another method would be analytical HPLC with a SEC column or analytical ultracentrifuge.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

All references mentioned herein are incorporated herewith by reference.

The following examples, sequences and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 Scheme of the DNA element according to the current invention prior (left sketch) and after (right sketch) RMCI.

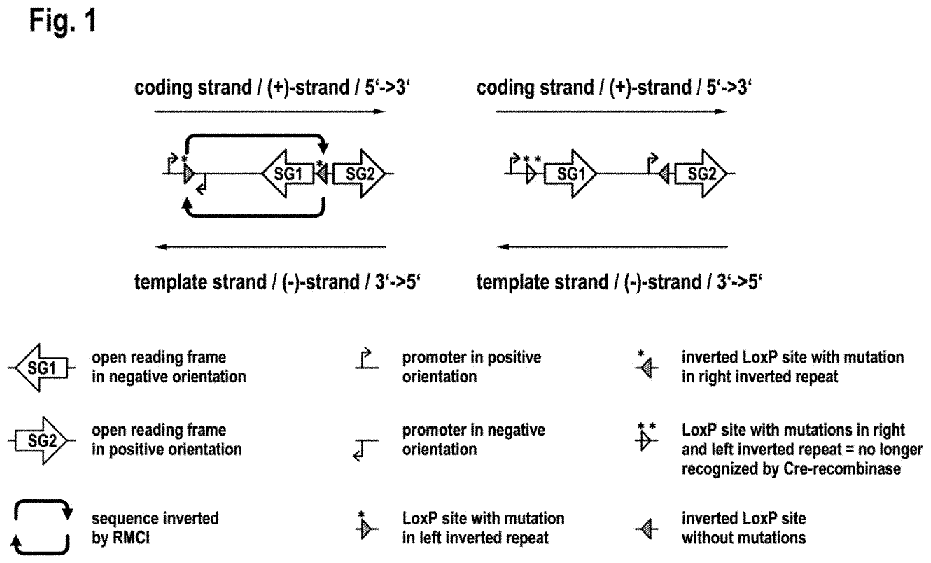

FIG. 2 Scheme of a DNA according to the current invention comprising two DNA elements according to the invention

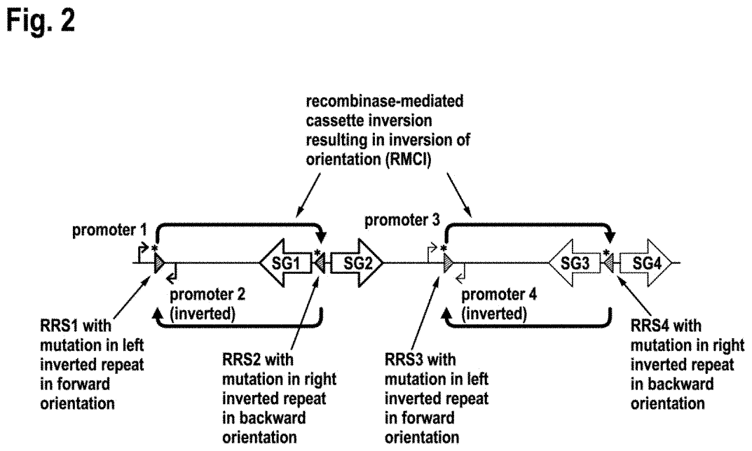

FIG. 3 Scheme of the DNA according to the current invention prior (upper sketch) and after (lower sketch) RMCI.

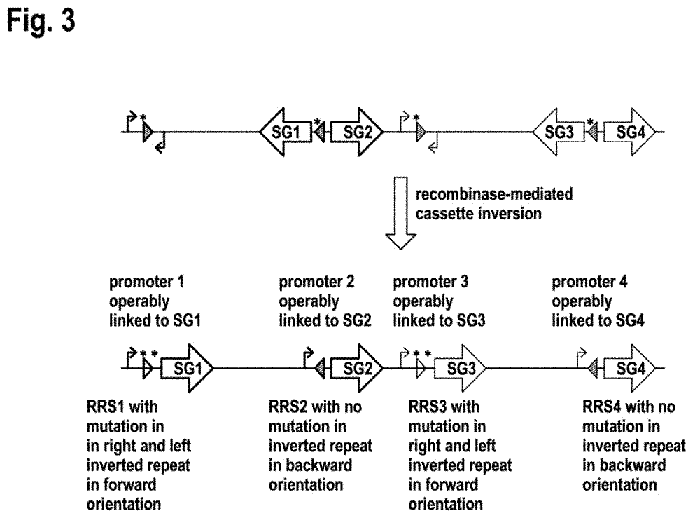

FIG. 4 Scheme of sequential transcription activation in a DNA according to the current invention.

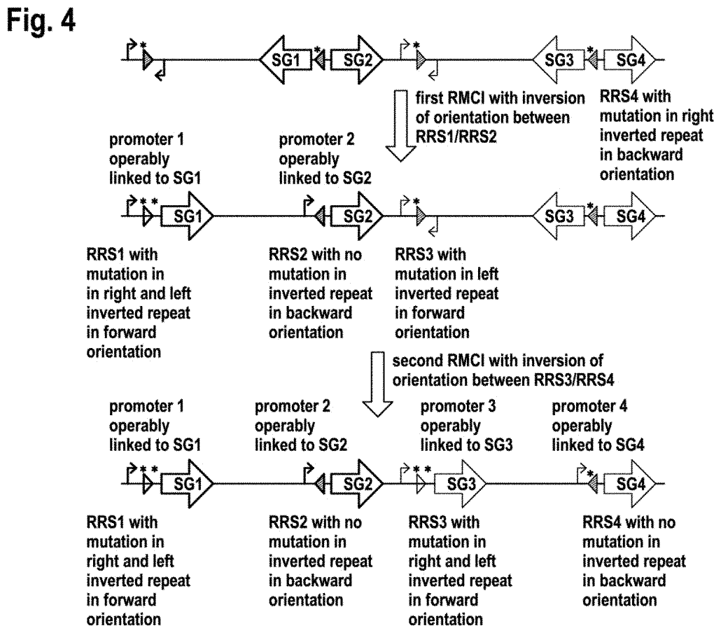

FIG. 5 Exemplary use of the DNA according to the current invention for simultaneous transcription activation of four open reading frames for E1A, E1B, E2A and E4(ORF6).

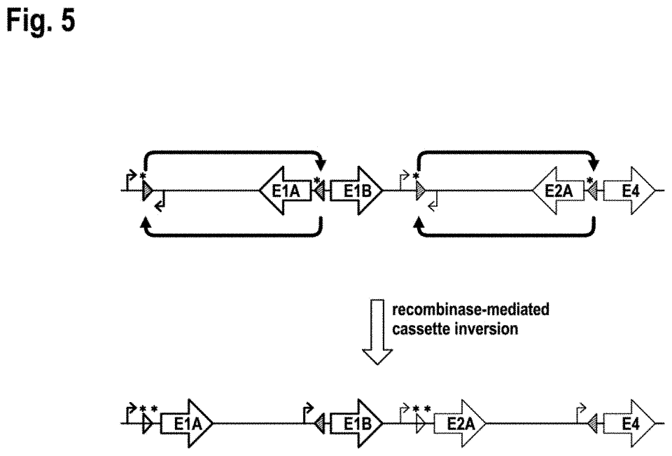

FIG. 6 Exemplary use of the DNA element according to the current invention for simultaneous transcription activation of two open reading frames for E2A and E4(ORF6).

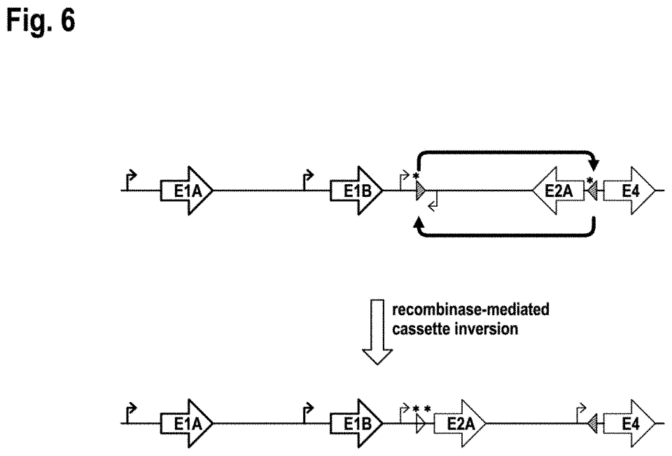

FIG. 7 Exemplary use of the DNA element according to the current invention for simultaneous transcription activation of two open reading frames for rep and cap (left), rep78 and rep52/40 (middle) and rep78 and rep52 (right).

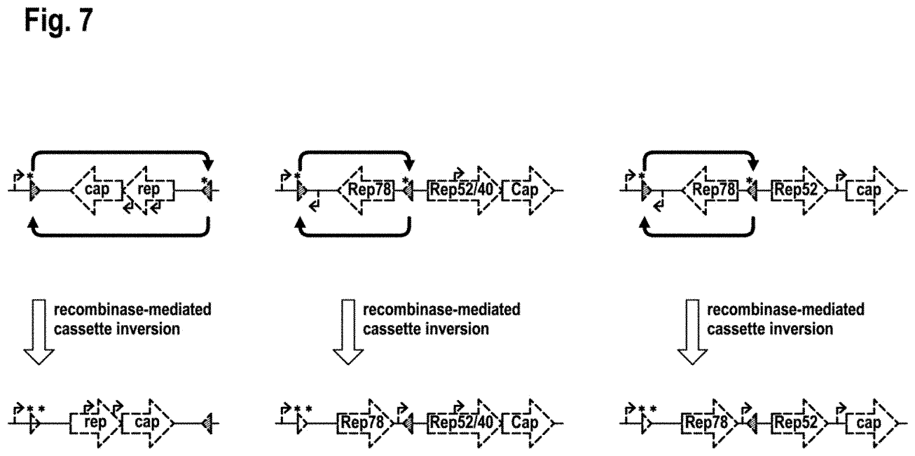

FIG. 8 Exemplary use of the DNA element according to the invention for transcription activation of one open reading frame for the VA RNA gene.

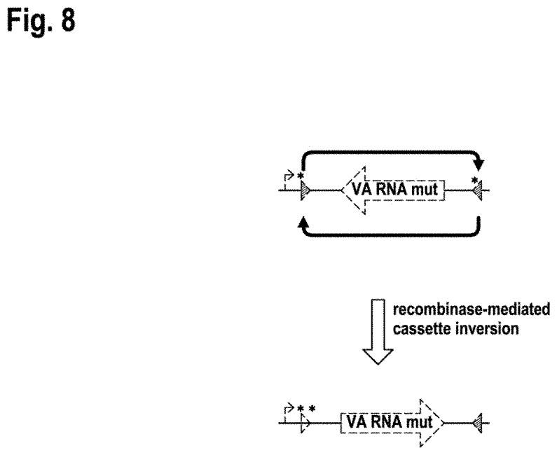

FIG. 9 Exemplary sketch of the DNA element according to the invention for simultaneous transcription active of the open reading frames for E1A and E1B prior to RMCI. The restriction sites for cloning are shown.

FIG. 10 Exemplary sketch of the inverted DNA element according to the invention with transcriptionally active open reading frames for E1A and E1B after RMCI. The restriction sites for cloning are shown.

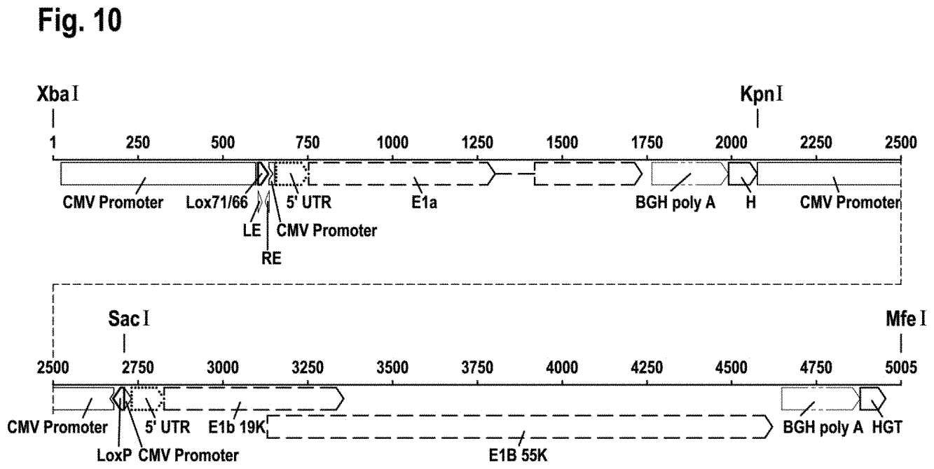

FIG. 11 Exemplary sketch of the DNA element according to the invention for simultaneous transcription active of the open reading frames for E2A and E4orf6 prior to RMCI. The restriction sites for cloning are shown.

FIG. 12 Exemplary sketch of the inverted DNA element according to the invention with transcriptionally active open reading frames for E2A and E4orf6 after RMCI. The restriction sites for cloning are shown.

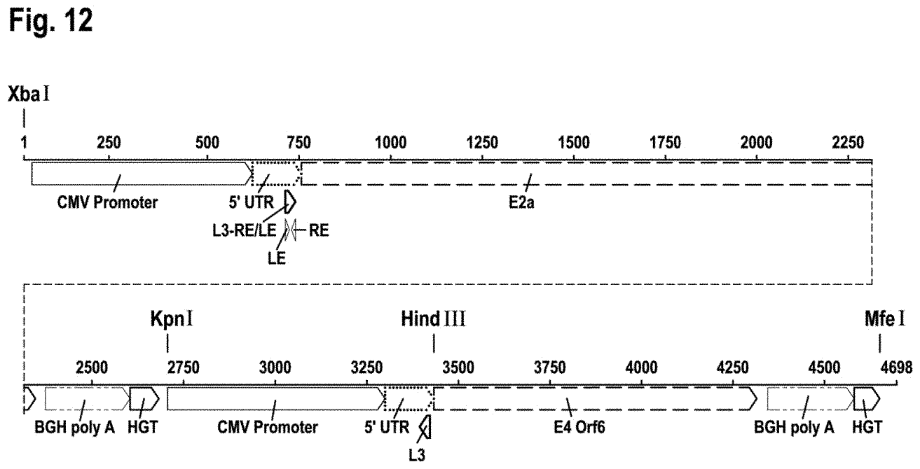

FIG. 13 Exemplary sketch of the DNA element according to the invention for simultaneous transcription active of the open reading frames for Rep78 and Rep52/40 prior to RMCI. The restriction sites for cloning are shown.

FIG. 14 Exemplary sketch of the inverted DNA element according to the invention with transcriptionally active open reading frames for Rep78 and Rep52/40 after RMCI. The restriction sites for cloning are shown.

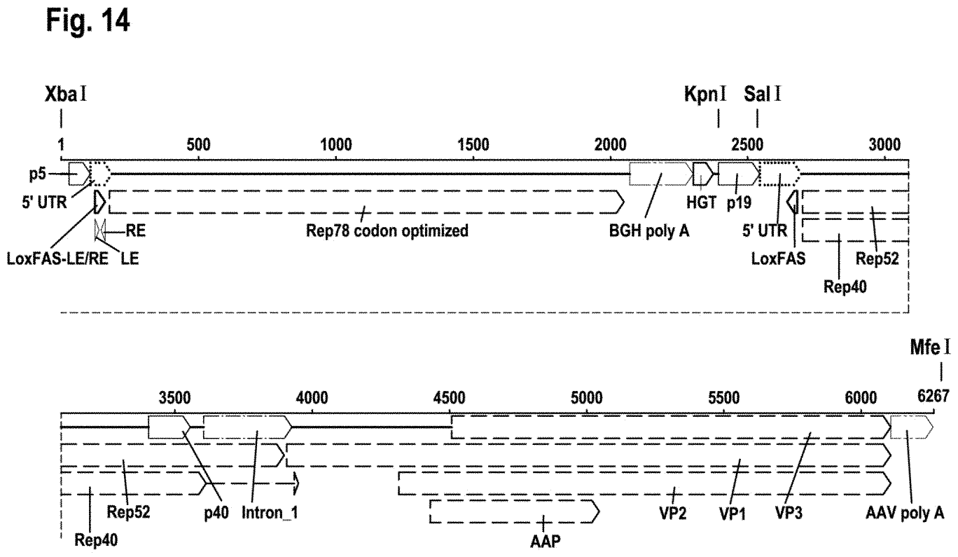

FIG. 15 Alignment of VA RNA and VA RNA G58T/G59T/C68A variant.

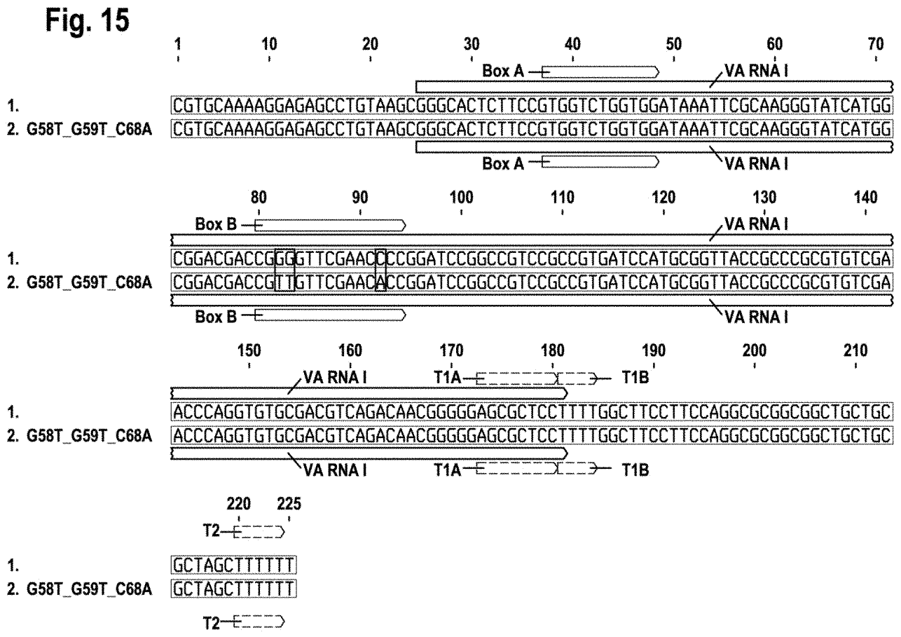

FIG. 16 VA RNA according to the current invention prior to RMCI.

FIG. 17 VA RNA according to the current invention after RMCI.

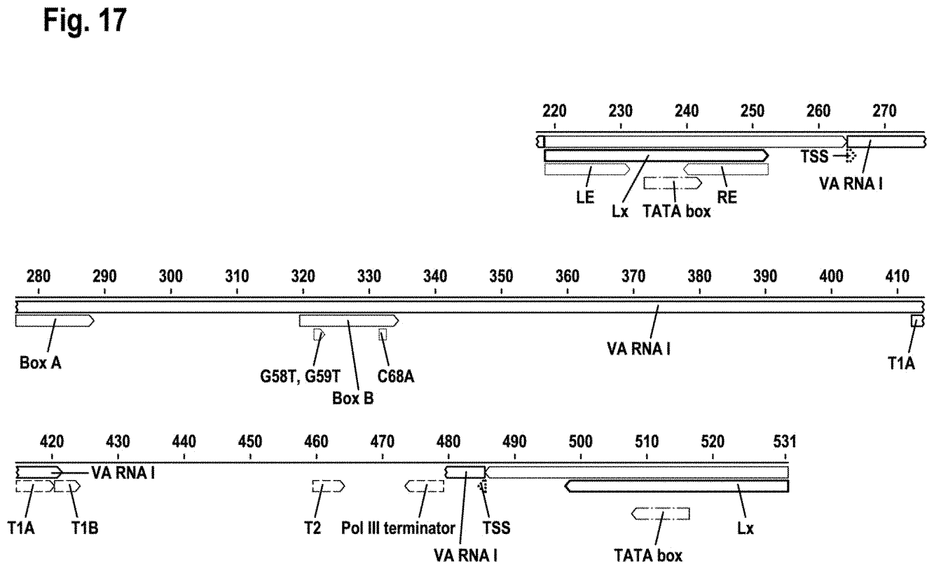

FIG. 18 Sketch of an exemplary, transcriptional inactive DNA element according to the invention for simultaneous transcriptional activation of the open reading frames for mCherry and EGFP prior to RMCI. The restriction sites for cloning are shown.

FIG. 19 Sketch of the inverted DNA element according to the invention of FIG. 18 with transcriptionally active open reading frames for mCherry and EGFP after RMCI. The restriction sites for cloning are shown.

FIG. 20 Cytometric analysis of RMCI in transiently transfected HEK293T cells. The mean percentage of GFP and mCherry expressing cells is shown together with the standard deviation (error bars). Each condition was tested in biological triplicates. Numbering according to Table 5.

### EXAMPLES

General Techniques

1) Recombinant DNA Techniques

Standard methods are used to manipulate DNA as described in Sambrook et al., Molecular Cloning: A Laboratory Manual, Second Edition, Cold Spring Harbor Laboratory Press, Cold Spring Harbor, N.Y, (1989). The molecular biological reagents are used according to the manufacturer's instructions.

2) DNA and Protein Sequence Analysis and Sequence Data Management

The EMBOSS (European Molecular Biology Open Software Suite) software package, Invitrogen's Vector NTI and Geneious Prime and are used for sequence creation, mapping, analysis, annotation and illustration.

3) Gene and Oligonucleotide Synthesis

Desired gene segments are prepared by chemical synthesis at Geneart GmbH (Regensburg, Germany). The synthesized gene fragments are cloned into an E. coli plasmid for propagation/amplification. The DNA sequences of subcloned gene fragments are verified by DNA sequencing. Alternatively, short synthetic DNA fragments are assembled by annealing chemically synthesized oligonucleotides or via PCR. The respective oligonucleotides are prepared by metabion GmbH (Planegg-Martinsried, Germany).

4) Reagents

All commercial chemicals, antibodies and kits are used as provided according to the manufacturer's protocol if not stated otherwise.

5) Cultivation of TI Host Cell Line

TI CHO host cells are cultivated at 37° C. in a humidified incubator with 85% humidity and 5% CO₂. They are cultivated in a proprietary DMEM/F12-based medium containing 300 μg/ml Hygromycin B and 4 μg/ml of a second selection marker. The cells are splitted every 3 or 4 days at a concentration of 0.3×10E6 cells/ml in a total volume of 30 ml. For the cultivation 125 ml non-baffle Erlenmeyer shake flasks are used. Cells are shaken at 150 rpm with a shaking amplitude of 5 cm.

The cell count is determined with Cedex HiRes Cell Counter (Roche). Cells are kept in culture until they reached an age of 60 days.

6) Cloning

General

Cloning with R-sites depends on DNA sequences next to the gene of interest (GOD that are equal to sequences lying in following fragments. Like that, assembly of fragments is possible by overlap of the equal sequences and subsequent sealing of nicks in the assembled DNA by a DNA ligase. Therefore, a cloning of the single genes in particular preliminary plasmids containing the right R-sites is necessary. After successful cloning of these preliminary plasmids the gene of interest flanked by the R-sites is cut out via restriction digest by enzymes cutting directly next to the R-sites. The last step is the assembly of all DNA fragments in one step. In more detail, a 5′-exonuclease removes the 5′-end of the overlapping regions (R-sites). After that, annealing of the R-sites can take place and a DNA polymerase extends the 3′-end to fill the gaps in the sequence. Finally, the DNA ligase seals the nicks in between the nucleotides. Addition of an assembly master mix containing different enzymes like exonucleases, DNA polymerases and ligases, and subsequent incubation of the reaction mix at 50° C. leads to an assembly of the single fragments to one plasmid. After that, competent E. coli cells are transformed with the plasmid.

For some plasmids, a cloning strategy via restriction enzymes was used. By selection of suitable restriction enzymes, the wanted gene of interest can be cut out and afterwards inserted into a different plasmid by ligation. Therefore, enzymes cutting in a multiple cloning site (MCS) are preferably used and chosen in a smart manner, so that a ligation of the fragments in the correct array can be conducted. If plasmid and fragment are previously cut with the same restriction enzyme, the sticky ends of fragment and plasmid fit perfectly together and can be ligated by a DNA ligase, subsequently. After ligation, competent E. coli cells are transformed with the newly generated plasmid.

Cloning Via Restriction Digestion

For the digest of plasmids with restriction enzymes the following components are pipetted together on ice:

TABLE
Restriction Digestion Reaction Mix

| component | ng (set point) | μl |
|---|---|---|
| purified DNA | tbd | tbd |
| CutSmart Buffer (10×) | | 5 |
| Restriction Enzyme | | 1 |
| PCR-grade Water | | ad 50 |
| Total | | 50 |

If more enzymes are used in one digestion, 1 μl of each enzyme is used and the volume is adjusted by addition of more or less PCR-grade water. All enzymes are selected on the preconditions that they are qualified for the use with CutSmart buffer from new England Biolabs (100% activity) and have the same incubation temperature (all 37° C.).

Incubation is performed using thermomixers or thermal cyclers, allowing incubating the samples at a constant temperature (37° C.). During incubation the samples are not agitated. Incubation time is set at 60 min. Afterwards the samples are directly mixed with loading dye and loaded onto an agarose electrophoresis gel or stored at 4° C./on ice for further use.

A 1% agarose gel is prepared for gel electrophoresis. Therefor 1.5 g of multi-purpose agarose are weighed into a 125 Erlenmeyer shake flask and filled up with 150 ml TAE-buffer. The mixture is heated up in a microwave oven until the agarose is completely dissolved. 0.5 μg/ml ethidium bromide are added into the agarose solution. Thereafter the gel is cast in a mold. After the agarose is set, the mold is placed into the electrophoresis chamber and the chamber is filled with TAE-buffer. Afterwards the samples are loaded. In the first pocket (from the left), an appropriate DNA molecular weight marker is loaded, followed by the samples. The gel is run for around 60 minutes at <130 V. After electrophoresis, the gel is removed from the chamber and analyzed in an UV-Imager.

The target bands are cut and transferred to 1.5 ml Eppendorf tubes. For purification of the gel, the QIAquick Gel Extraction Kit from Qiagen is used according to the manufacturer's instructions. The DNA fragments are stored at −20° C. for further use.

The fragments for the ligation are pipetted together in a molar ratio of 1:2, 1:3 or 1:5 plasmid to insert, depending on the length of the inserts and the plasmid-fragments and their correlation to each other. If the fragment, that should be inserted into the plasmid is short, a 1:5-ratio is used. If the insert is longer, a smaller amount of it is used in correlation to the plasmid. An amount of 50 ng of plasmid is used in each ligation and the particular amount of insert calculated with NEBioCalculator. For ligation, the T4 DNA ligation kit from NEB is used. An example for the ligation mixture is depicted in the following Table.

TABLE
Ligation Reaction Mix

| component | ng (set point) | conc. [ng/μl] | μl |
|---|---|---|---|
| T4 DNA Ligase | | | 2 |
| Buffer (10×) | | | |
| Plasmid DNA (4000 bp) | 50 | 50 | 1 |
| Insert DNA (2000 bp) | 125 | 20 | 6.25 |
| Nuclease-free Water | | | 9.75 |
| T4 Ligase | | | 1 |
| Total | | | 20 |

All components are pipetted together on ice, starting with the mixing of DNA and water, addition of buffer and finally addition of the enzyme. The reaction is gently mixed by pipetting up and down, briefly microfuged and then incubated at room temperature for 10 minutes. After incubation, the T4 ligase is heat inactivated at 65° C. for 10 minutes. The sample is chilled on ice. In a final step, 10-beta competent E. coli cells are transformed with 2 μl of the ligated plasmid (see below).

Transformation 10-Beta Competent E. coli Cells

For transformation, the 10-beta competent E. coli cells are thawed on ice. After that, 2 μl of plasmid DNA is pipetted directly into the cell suspension. The tube is flicked and put on ice for 30 minutes. Thereafter, the cells are placed into a 42° C. thermal block and heat-shocked for exactly 30 seconds. Directly afterwards, the cells are chilled on ice for 2 minutes. 950 μl of NEB 10-beta outgrowth medium are added to the cell suspension. The cells are incubated under shaking at 37° C. for one hour. Then, 50-100 μl are pipetted onto a pre-warmed (37° C.) LB-Amp agar plate and spread with a disposable spatula. The plate is incubated overnight at 37° C. Only bacteria, which have successfully incorporated the plasmid, carrying the resistance gene against ampicillin, can grow on these plates. Single colonies are picked the next day and cultured in LB-Amp medium for subsequent plasmid preparation.

Bacterial Culture

Cultivation of E. coli is done in LB-medium, short for Luria Bertani, which is spiked with 1 ml/L 100 mg/ml ampicillin resulting in an ampicillin concentration of 0.1 mg/ml. For the different plasmid preparation quantities, the following amounts are inoculated with a single bacterial colony.

TABLE
E. coli cultivation volumes

| Quantity plasmid | Volume LB-Amp | Incubation |
|---|---|---|
| preparation | medium [ml] | time [h] |
| Mini-Prep 96-well (EpMotion) | 1.5 | 23 |
| Mini-Prep 15 ml-tube | 3.6 | 23 |
| Maxi-Prep | 200 | 16 |

For Mini-Prep, a 96-well 2 ml deep-well plate is filled with 1.5 ml LB-Amp medium per well. The colonies are picked and the toothpick is tuck in the medium. When all colonies are picked, the plate is closed with a sticky air porous membrane. The plate is incubated in a 37° C. incubator at a shaking rate of 200 rpm for 23 hours.

For Mini-Preps a 15 ml-tube (with a ventilated lid) is filled with 3.6 ml LB-Amp medium and equally inoculated with a bacterial colony. The toothpick is not removed but left in the tube during incubation. Like the 96-well plate, the tubes are incubated at 37° C., 200 rpm for 23 hours.

For Maxi-Prep 200 ml of LB-Amp medium are filled into an autoclaved glass 1 L Erlenmeyer flask and are inoculated with 1 ml of bacterial day-culture, that is roundabout 5 hours old. The Erlenmeyer flask is closed with a paper plug and incubated at 37° C., 200 rpm for 16 hours.

Plasmid Preparation

For Mini-Prep, 50 μl of bacterial suspension are transferred into a 1 ml deep-well plate. After that, the bacterial cells are centrifuged down in the plate at 3000 rpm, 4° C. for 5 min. The supernatant is removed and the plate with the bacteria pellets is placed into an EpMotion. After approx. 90 minutes, the run is done and the eluted plasmid-DNA can be removed from the EpMotion for further use.

For Mini-Prep, the 15 ml tubes are taken out of the incubator and the 3.6 ml bacterial culture is splitted into two 2 ml Eppendorf tubes. The tubes are centrifuged at 6,800×g in a tabletop microcentrifuge for 3 minutes at room temperature. After that, Mini-Prep is performed with the Qiagen QIAprep Spin Miniprep Kit according to the manufacturer's instructions. The plasmid DNA concentration is measured with Nanodrop.

Maxi-Prep is performed using the Macherey-Nagel NucleoBond® Xtra Maxi EF Kit according to the manufacturer's instructions. The DNA concentration is measured with Nanodrop.

Ethanol Precipitation

The volume of the DNA solution is mixed with the 2.5-fold volume ethanol 100%. The mixture is incubated at −20° C. for 10 min. Then the DNA is centrifuged for 30 min. at 14,000 rpm, 4° C. The supernatant is carefully removed and the pellet is washed with 70% ethanol. Again, the tube is centrifuged for 5 min. at 14,000 rpm, 4° C. The supernatant is carefully removed by pipetting and the pellet is dried. When the ethanol is evaporated, an appropriate amount of endotoxin-free water is added. The DNA is given time to re-dissolve in the water overnight at 4° C. A small aliquot is taken and the DNA concentration is measured with a Nanodrop device.

Expression Cassette Composition

For the expression of an open reading frame, a transcription unit comprising the following functional elements is used:
the immediate early enhancer and promoter from the human cytomegalovirus including intron A,
a human heavy chain immunoglobulin 5′-untranslated region (5′UTR),
a nucleic acid comprising the respective open reading frame including signal sequences, if required,
the bovine growth hormone polyadenylation sequence (BGH pA), and
optionally the human gastrin terminator (hGT).

Beside the expression unit/cassette including the desired gene to be expressed, the basic/standard mammalian expression plasmid contains
an origin of replication from the plasmid pUC18 which allows replication of this plasmid in E. coli, and
a beta-lactamase gene which confers ampicillin resistance in E. coli.

Cell Culture Techniques

Standard cell culture techniques are used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

Transient Transfections in HEK293 System

Cells comprising the DNA elements according to the current invention are generated by transient transfection with the respective plasmids (see Examples 1 to 4 below) using the HEK293 system (Invitrogen) according to the manufacturer's instruction. Briefly, HEK293 cells (Invitrogen) growing in suspension either in a shake flask or in a stirred fermenter in serum-free FreeStyle™ 293 expression medium (Invitrogen) are transfected with a mix of the respective plasmids and 293Fectin™ or fectin (Invitrogen). For 2 L shake flask (Corning) HEK293 cells are seeded at a density of 1*10⁶ cells/mL in 600 mL and are incubated at 120 rpm, 8% CO₂. The day after the cells are transfected at a cell density of ca. 1.5*10⁶ cells/mL with ca. 42 mL mix of A) 20 mL Opti-MEM (Invitrogen) with 600 μg total plasmid DNA (1 μg/mL) and B) 20 ml Opti-MEM+1.2 mL 293 fectin or fectin (2 μL/mL). According to the glucose consumption, glucose solution is added during the course of the fermentation.

SDS-PAGE

LDS sample buffer, fourfold concentrate (4×): 4 g glycerol, 0.682 g TRIS-Base, 0.666 g TRIS-hydrochloride, 0.8 g LDS (lithium dodecyl sulfate), 0.006 g EDTA (ethylene diamine tetra acid), 0.75 ml of a 1% by weight (w/w) solution of Serva Blue G250 in water, 0.75 ml of a 1% by weight (w/w) solution of phenol red, add water to make a total volume of 10 ml.

The cells in the culture broth are lysed. Thereafter the solution was centrifuged to remove cell debris. An aliquot of the clarified supernatant is admixed with 1/4 volumes (v/v) of 4×LDS sample buffer and 1/10 volume (v/v) of 0.5 M 1,4-dithiotreitol (DTT). Then the samples are incubated for 10 min. at 70° C. and protein separated by SDS-PAGE. The NuPAGE® Pre-Cast gel system (Invitrogen Corp.) was used according to the manufacturer's instruction. In particular, 10% NuPAGE® Novex® Bis-TRIS Pre-Cast gels (pH 6.4) and a NuPAGE® MOPS running buffer was used.

Western Blot

Transfer buffer: 39 mM glycine, 48 mM TRIS-hydrochloride, 0.04% by weight (w/w) SDS, and 20% by volume methanol (v/v)

After SDS-PAGE the separated polypeptides were transferred electrophoretically to a nitrocellulose filter membrane (pore size: 0.45 μm) according to the “Semidry-Blotting-Method” of Burnette (Burnette, W. N., Anal. Biochem. 112 (1981) 195-203).

### Example 1

Generation of a DNA Construct for Simultaneous Cre-Recombinase Mediated Activation of E2A and E4orf6 Open Reading Frames by RMCI According to the Invention

A first DNA fragment is generated wherein the 608 bp CMV immediate early promoter and enhancer (SEQ ID NO: 28) is combined with a human immunoglobulin 5′ UTR. Two such elements are fused head to head with an intermitting L3 element with mutated left inverted repeat (L3-LE; taccgttcgt ataaagtctc ctatacgaag ttat; SEQ ID NO: 70) and flanked with an XbaI (5′-end) and a KpnI (3′-end) restriction site. The corresponding DNA fragment is generated by DNA synthesis and cloned into a suitable shuttle plasmid.

Likewise a second DNA fragment is generated and cloned, comprising in 5′- to 3′-direction with respect to its coding strand: a HindIII restriction site, an L3 site with mutated right inverted repeat (L3-RE; ataacttcgt ataaagtctc ctatacgaac ggta; SEQ ID NO: 71), a Kozak sequence, an open reading frame coding for the adenoviral E2A protein (GenBank accession number AC_000007), the bovine growth hormone polyadenylation signal sequence (BGH poly A; SEQ ID NO: 31), the human gastrin transcription terminator sequence (HGT; SEQ ID NO: 32) and a KpnI restriction site.

A third fragment is generated and cloned as well, comprising in 5′- to 3′-direction with respect to its coding strand: an MfeI restrictions site, a Kozak sequence, an open reading frame coding for the adenoviral E4orf6 protein (GenBank accession number AC_000007), the BGH poly A, the HGT sequence and a HindIII restriction site.

The three fragments are excised from their shuttle plasmids using the respective restriction enzymes. The excised fragments are combined with a plasmid backbone carrying MfeI- and XbaI-compatible overhangs and a puromycin selection marker in a four-way ligation reaction, yielding a plasmid for stable transfection of mammalian cells.

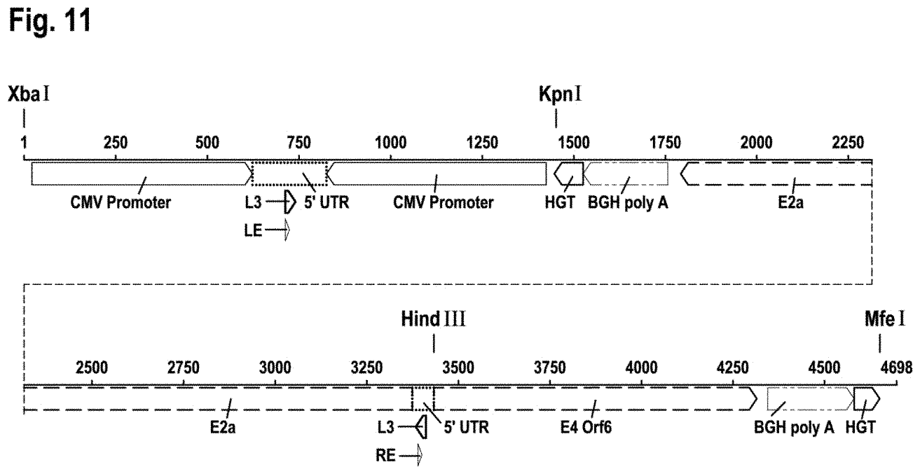

FIG. 11 illustrates the order and orientation of the elements within this DNA fragment, which is determined by the compatibility of sticky ends during ligation.

### Example 2

Generation of a DNA Construct for Simultaneous Cre-Recombinase Mediated Activation of E1A and E1B Open Reading Frames by RMCI According to the Invention

Two copies of the 608 bp CMV promoter and enhancer element but excluding the sequence between the TATA box and the transcription start site are fused head to head with an intermitting Lox71 site. The resulting fragment is provided with an XbaI restriction site at the 5′-end and a KpnI restriction site at the 3′-end. The complete DNA fragment is generated by DNA synthesis and cloned into a suitable shuttle plasmid.

Likewise a second DNA fragment is synthesized and cloned, comprising in 5′- to 3′-direction with respect to its coding strand: a SacI restriction site, a Lox66 site, the CMV promoter fragment between the TATA box and the transcription initiation site with mutated/inactivated SacI site, a human immunoglobulin heavy chain 5′UTR, a Kozak sequence, an open reading frame coding for the adenoviral E1A protein (GenBank accession number AC_000008), the bovine growth hormone polyadenylation signal sequence (BGH poly A), the human gastrin transcription terminator sequence (HGT) and a KpnI restriction site.

A third fragment is synthesized and cloned as well, comprising in 5′- to 3′-direction: a SacI restriction site, the CMV promoter fragment between the TATA box and the transcription initiation site, a human immunoglobulin heavy chain 5′-UTR, a Kozak sequence, open reading frames coding for the adenoviral E1B 19 kDa and E1B 55 kDa proteins (GenBank accession number AC_000008), the bovine growth hormone polyadenylation signal sequence (BGH poly A), the human gastrin transcription terminator sequence (HGT) and an MfeI restriction site.

The three fragments are excised from their shuttle plasmids using the respective restriction enzymes. The fragments are combined with a plasmid backbone carrying MfeI- and XbaI-compatible overhangs and a puromycin selection marker in a four-way ligation reaction, yielding a plasmid for stable transfection of mammalian cells.

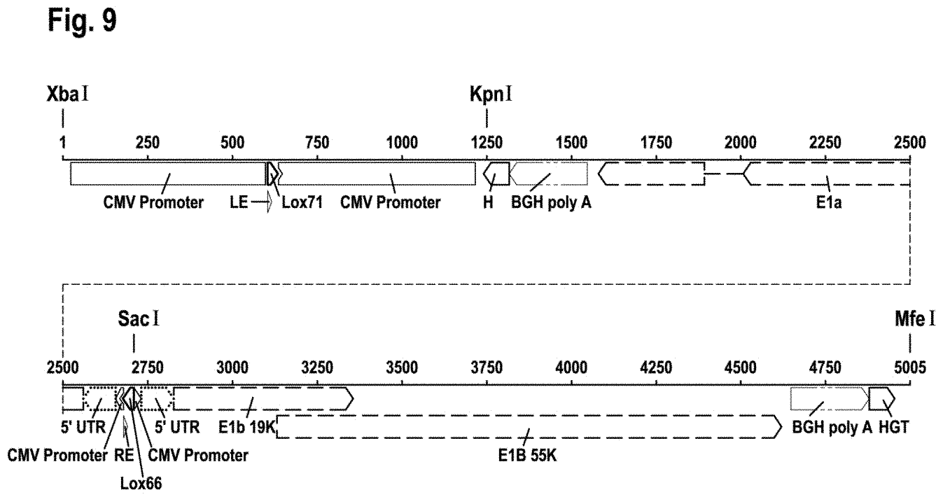

FIG. 9 illustrates the order and orientation of the elements within this DNA fragment, which is determined by the compatibility of sticky ends during ligation.

### Example 3

Generation of a DNA Construct for Simultaneous Cre-Recombinase Mediated Activation of Rep78 and Rep52/40 Transcription by RMCI According to the Invention

The AAV2 P5 promoter including 21 bp downstream of the transcription start site and the AAV2 P19 promoter including 103 bp downstream of the transcription start site are fused head-to-head with an intermitting LoxFas site with mutated left inverted repeat (LoxFas-LE; taccgttcgt atataccttt ctatacgaag ttat; SEQ ID NO: 72). The resulting fragment is provided with an XbaI restriction site at the 5′-end and a KpnI restriction site at the 3′-end. The complete DNA fragment is generated by DNA synthesis and cloned in a suitable shuttle plasmid.

Likewise a second DNA fragment is generated and cloned, comprising in 3′- to 5′-direction, i.e. inverted with respect to the coding strand: a SalI restriction site, a LoxFas site with mutated right inverted repeat (LoxFas-RE; ataacttcgt atataccttt ctatacgaac ggta; SEQ ID NO: 73), a 13 bp sequence from the Rep78/68 5′UTR, an open reading frame coding for the AAV2 Rep78 protein, the bovine growth hormone polyadenylation signal (BGH poly A), the human gastrin transcription terminator (HGT) and a KpnI restriction site.

A third fragment is generated as well comprising in 5′- to 3′-direction: a SalI restriction site, the AAV2 Rep52/40-Cap gene starting at 13 bp upstream of the Rep52/40 start codon and ending at 124 bp downstream of the stop codon of the VP genes and an Mfe restriction site.

The three fragments are excised from their shuttle plasmids using the respective restriction enzymes. The fragments are combined with a plasmid backbone carrying MfeI and XbaI-compatible overhangs and a puromycin selection marker in a four-way ligation reaction, yielding a plasmid for stable transfection of mammalian cells.

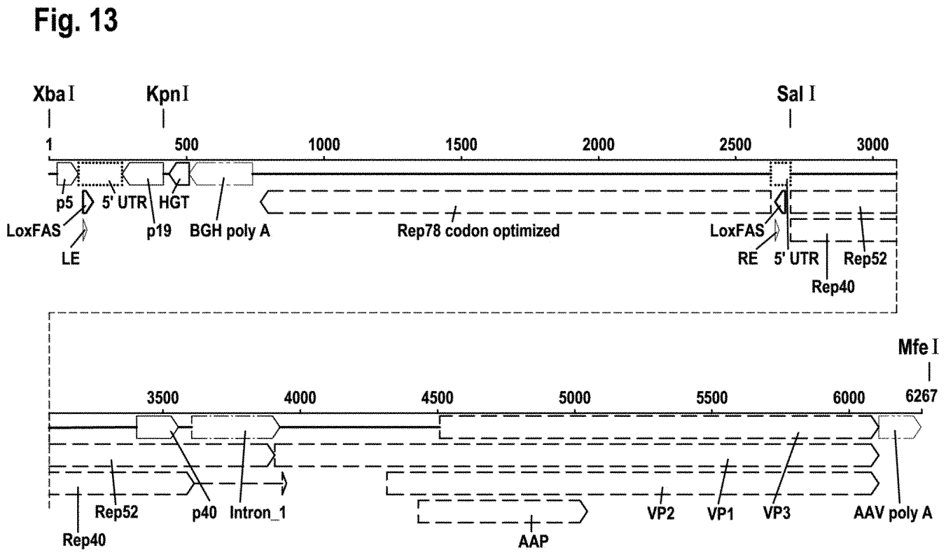

FIG. 13 illustrates the order and orientation of the elements within this DNA fragment, which is determined by the compatibility of sticky ends during ligation.

### Example 4

Generation of a DNA Construct for Cre-Recombinase Mediated Activation of VA RNAI Transcription by RMCI According to the Invention

A DNA fragment is chemically synthesized comprising in 5′- to 3′-direction: an Lx-LE site of SEQ ID NO: 69 comprising a TATA signal (TTTATATAT; SEQ ID NO: 74) integrated into a Cre-recombination site with mutated left inverted repeat and high divergence from the canonical LoxP site ensuring non-promiscuity (Lx-LE; taccgttcgt ataagtttat atatacgaag ttat; SEQ ID NO: 03) (the distance between TATA and the transcription start site is aligned to reflect the general distance), a short fragment from the very 5′-end of the Ad2 VA RNAI gene immediately followed by a polymerase III terminator (hexa-dT), the Ad2 VA RNAI gene (GenBank AC_000007) in reverse orientation as well as a 3′ terminal sequence comprising a Lx site with right inverted repeat in reverse orientation (Lx-RE reverse; taccgttcgt atatataaac ttatacgaag ttat; SEQ ID NO: 06).

The fragment is ligated with a plasmid backbone carrying a puromycin selection marker, yielding a plasmid for stable transfection of mammalian cells.

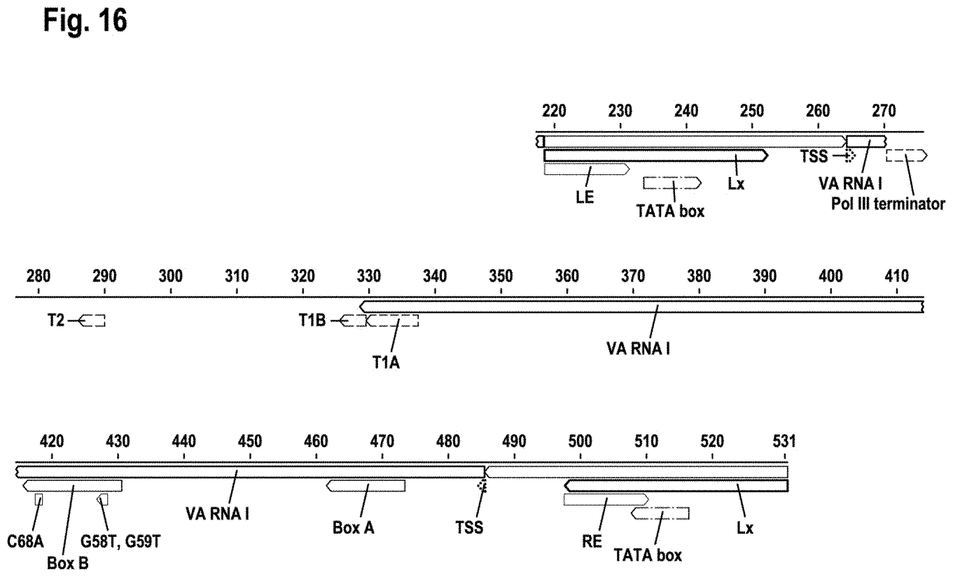

FIG. 16 illustrates the order and orientation of the elements within this DNA fragment.

### Example 5

Stable Integration of Cassettes for RMCI

CHO-K1 cells, adapted to grow in suspension, are propagated in 50 mL chemically defined medium in disposable, vented 125 mL shake flasks at 37° C. and 5-7 vol.-% CO₂. The cultures are shaken with a constant agitation rate of 140-180 rpm/min and diluted every 3-4 days to a density of 2-3×10⁵/mL with fresh medium. The density and viability of the cultures are determined using Cedex HiRes cell counter (Roche Innovates AG, Bielefeld, Germany).

For stable integration of RMCI cassettes, the suspension-growing CHO-K1 cells are seeded in fresh chemically defined medium with a density of 4×10⁵ cells/mL. On the following day, transfection is performed with the Nucleofector device using the Nucleofector Kit V (Lonza, Switzerland) according to the manufacturer's protocol. 3×10⁷ cells are transfected with 30 μg linearized plasmid DNA. After transfection, the cells are seeded in 30 ml fresh chemically defined medium without selection agents.

Two days after transfection, cells are seeded into 384-well plates containing 1 to 10 μg/mL puromycin as selection agent with 300 to 500 cells per well. After three weeks, cell colonies are identified by imaging using a NYONE Plate imager (SYNENTECH GmbH, Elmshom, Germany). Colonies are transferred to 96-well plates and analyzed for the integration of the RMCI cassettes by PCR. Cell lines containing all desired RMCI cassettes are further expanded in chemically defined medium containing puromycin and are cryo-preserved after expansion.

### Example 6

Gene Activation and AAV Production by Cre-Mediated Cassette Inversion (RMCI) According to the Invention

Gene Activation by Cre-Recombinase-Mediated RMCI

For Cre-mediated gene activation by cassette inversion (Cre-mediated RMCI), cells carrying either inactive RMCI cassettes of adenoviral helper genes and/or the rep-cap gene as obtained in one of the examples above are transiently transfected with Cre-recombinase encoding mRNA. One day prior to transfection, cells are seeded in fresh medium with a density of 4×10⁵ cells/mL. On the following day, transfection is performed with the Nucleofector device using the Nucleofector Kit V (Lonza, Switzerland) according to the manufacturer's protocol. 3×10⁷ cells are transfected with a total amount of 30 μg Cre-recombinase encoding mRNA. Successful gene activation is proven by PCR of the inverted genomic DNA, RT-PCR of the expected mRNA or Western blot analysis of the expected gene product.

Generation of rAAV Vector Producing Cells

For the production of recombinant AAV vectors, 3×10⁷ cells carrying either inactive RMCI cassettes of adenoviral helper genes and/or the rep-cap gene as obtained in one of the examples above are transiently transfected with a total amount of 30 pg nucleic acid comprising 5 μg Cre-recombinase encoding mRNA. The remaining 25 pg nucleic acid is composed of plasmid DNA providing a recombinant AAV genome (transgene, e.g. a GFP gene flanked by AAV ITRs) and expression cassettes for helper genes and/or the rep/cap gene that have not been integrated into the genome.

Alternatively, the recombinant AAV genome is provided by stable integration into the genome of the host cell as described in Example 5.

If the cells' genome already comprises all essential helper genes, rep/cap and a recombinant AAV genome, transfection of Cre-recombinase encoding mRNA alone is sufficient.

AAV particles are harvested from the cell culture supernatant or the total cell lysate and are analyzed by ELISA, quantitative PCR and transduction of target cells.

### Example 7

Generation of a DNA Construct for Simultaneous FRT-Recombinase Mediated Activation of mCherry and EGFP Open Reading Frames by RMCI According to the Invention

A first DNA fragment was generated wherein a 52 bp minimal CMV promoter (SEQ ID NO: 85) was combined in its transcriptional direction with the following elements in the following order:
a human immunoglobulin 5′ UTR;
an FRT element with mutated left inverted repeat (FRT-LE; GAAGTTCATATTCTCTAGAAAGTATAGGAACTTC; SEQ ID NO: 60);
a 417 bp fragment of the SV40 early promoter including the transcription start (TS) region (SEQ ID NO: 86) in reverse orientation;
the human gastrin transcription terminator sequence (HGT) of SEQ ID NO: 32 but in reverse orientation;
the bovine growth hormone polyadenylation signal sequence (BGH poly A) of SEQ ID NO: 31 but in reverse orientation;
an open reading frame coding for the mCherry fluorescent protein (GenBank accession number QUW04963; SEQ ID NO: 87) but in reverse orientation;
a Kozak sequence but in reverse orientation;
an FRT site with mutated right inverted repeat (FRT-RE; GAAGTTCCTATTCTCTAGAAAGTATATGAACTTC, SEQ ID NO: 61) but in reverse orientation;
a Kozak sequence in forward orientation; and
the 5′-part part of an open reading frame coding for the enhanced green fluorescent protein (EGFP; GenBank accession number AAB02572.1; SEQ ID NO: 88; 26 bp) in forward orientation.

The corresponding DNA fragment was flanked with a SalI (at the 5′-end) and a SgrAI (at the 3′-end) restriction site, generated by DNA synthesis and cloned into a suitable shuttle plasmid.

A second fragment was generated and cloned as well, comprising in 5′- to 3′-direction in the following order with respect to its coding strand: a SalI restriction site, an open reading frame coding for EGFP and comprising an internal SgrAI restriction site, the BGH poly A signal sequence, the HGT sequence and a MfeI restriction site.

The first fragment was excised from its shuttle plasmids using SalI and SgrAI restriction enzymes and inserted between the SalI and SgrAI sites of the plasmid carrying the second fragment, yielding the final plasmid, which is suitable for transient transfection of mammalian cells.

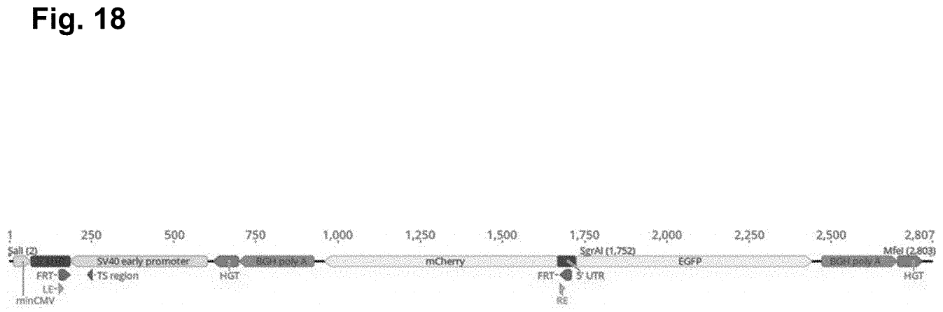

FIG. 18 illustrates the order and orientation of the elements within the combined DNA of the first and the second fragment.

### Example 8—Comparative Example

Generation of a DNA Construct Representing the DNA Configuration to be Obtained after Simultaneous FRT-Recombinase Mediated Activation of mCherry and EGFP Open Reading Frames by RMCI According to the Invention

A first DNA fragment was generated wherein a 52 bp minimal CMV promoter (SEQ ID NO: 85) is combined in its transcriptional direction in the following order with:
a human immunoglobulin 5′ UTR in forward orientation;
an FRT element with mutated left and right inverted repeats (FRT-LE-RE; GAAGTTCATATTCTCTAGAAAGTATATGAACTTC; SEQ ID NO: 89) in forward orientation;
a Kozak sequence in forward orientation;
an open reading frame coding for the mCherry fluorescent protein (GenBank accession number QUW04963; SEQ ID NO: 87) in forward orientation;
the bovine growth hormone polyadenylation signal sequence (BGH poly A; SEQ ID NO: 31) in forward orientation;
the human gastrin transcription terminator sequence (HGT; SEQ ID NO: 32) in forward orientation;
a 417 bp fragment of the SV40 early promoter including the transcription start (TS) region (SEQ ID NO: 86) in forward orientation;
an FRT site of SEQ ID NO: 36 but in reverse orientation;
a Kozak sequence in forward orientation; and
the 5′-part part of an open reading frame coding for the enhanced green fluorescent protein (EGFP; GenBank accession number AAB02572.1; SEQ ID NO: 88; 26 bp) in forward orientation.

The corresponding DNA fragment was flanked with a SalI (at the 5′-end) and a SgrAI (at the 3′-end) restriction site, generated by DNA synthesis and cloned into a suitable shuttle plasmid.

The first fragment was excised from its shuttle plasmid using SalI and SgrAI restriction enzymes and inserted between the SalI and SgrAI sites of the plasmid carrying the second fragment as described in Example 7, yielding a plasmid for transient transfection of mammalian cells.

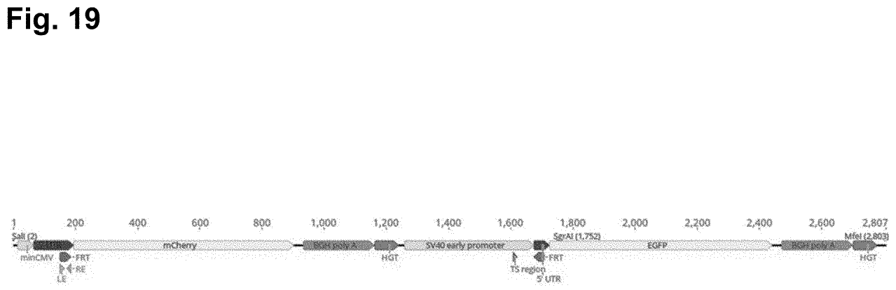

FIG. 19 illustrates the order and orientation of the elements within the combined DNA of the first and the second fragment.

### Example 9

Simultaneous Activation of Two Fluorescence Genes by FLP-Mediated Cassette Inversion (RMCI) According to the Invention

Transfection

HEK293T adherent cells were cultivated in DMEM, high glucose, GlutaMAX™ Supplement, pyruvate medium (Thermo Fisher Scientific) supplemented with 10% fetal bovine serum (Thermo Fisher Scientific) at 37° C., 90% relative humidity and 5% CO₂. Twenty-four hours prior to transfection, 10,000 cells per well were seeded in the wells of a 96 well plate. Cells were transfected with mixtures of 100 ng plasmid DNA per well using PEI max (Polyscience) at a DNA to PEI max ratio of 1:2 according to the manufacturer's recommendations. Each experimental condition as shown in the following Table 5 was tested in triplicates.

TABLE 5
Composition of plasmid mixtures for transfection. DNA amounts in ng per well are indicated for each experimental condition (1 to 14).

| ↓Plasmid\Condition→ | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| FLPo (encoding FPL recombinase) | | | | 20 | 10 | 5 | | | 20 | 10 | 5 | 20 | 10 | 5 |
| mCherry_EGFP_pre rec (obtained according to Example 7) | | | | | | | 80 | | 80 | 80 | 80 | | | |
| mCherry_EGFP_post rec (obtained according to Example 8) | | | | | | | | 80 | | | | 80 | 80 | 80 |
| Mock DNA | 100 | 20 | 20 | 80 | 90 | 95 | 20 | 20 | | 10 | 15 | | 10 | 15 |
| reference: EGFP_only | | 80 | | | | | | | | | | | | |
| reference: mCherry_only | | | 80 | | | | | | | | | | | |

In order to demonstrate the simultaneous activation of mCherry and EGFP genes by FLP-recombinase-mediated cassette inversion according to the invention, 80 ng of the inactive construct mCherry_EGFP_pre rec (Example 7, FIG. 18) was mixed with variable amounts of a plasmid coding for the FPL recombinase FLPo, which is an optimized version of FLP recombinase (see, e.g., Raymond, .CS. and Soriano, P. PLoS ONE 2 (2007) e162). Non-coding plasmid (mock DNA) was added as needed to keep the total amount of DNA in the transfection mixture at 100 ng. The corresponding conditions were applied for the active construct mCherry_EGFP_post rec (Example 8, FIG. 19) in order to test whether or not the expression of mCherry and EGFP is affected by co-expression of FLPo.

Mock DNA alone was transfected as negative control whereas EGFP or mCherry expressing single gene plasmids (EGFP_only and mCherry_only) serve as positive control. FLPo plasmid in combination with mock DNA was transfected to exclude any direct induction of fluorescence by FLPo alone.

Flow Cytometry

Two days after transient transfection, the success of FLP-mediated cassette inversion was checked by flow cytometry measuring the expression of intracellular EGFP and mCherry. To this end, HEK293T cells were harvested from a 96-well plate by trypsin-mediated detachment. The reaction was stopped by the addition of 2% fetal bovine serum in phosphate buffered saline.

Flow cytometry was performed with a BD FACSCelesta™ Flow Cytometer (BD, Heidelberg, Germany). Living cells were gated in a plot of forward scatter (FSC) against side scatter (SSC). To distinguish between singlets and cell aggregates a FSC-H vs FASC-A plot was chosen. Ten thousand events per sample were recorded. Both gates were defined with mock-transfected HEK293T cells and applied to all samples by employing the FlowJo v10.6.2 software (TreeStar, Olten, Switzerland). Fluorescence of GFP was quantified in the FITC channel (excitation at 488 nm, detection at 530 nm). mCherry was measured in the PE-CF594 channel (excitation at 561 nm, detection at 610 nm).

To correctly identify the fluorescent cell population and to adjust the lasers, positive and negative control samples were used. Cells transfected with EGFP_only plasmid were used as the EGFP positive control and cells transfected with mCherry_only plasmid were used as the mCherry positive control. Cells transfected with non-coding plasmid (mock DNA) served as negative control.

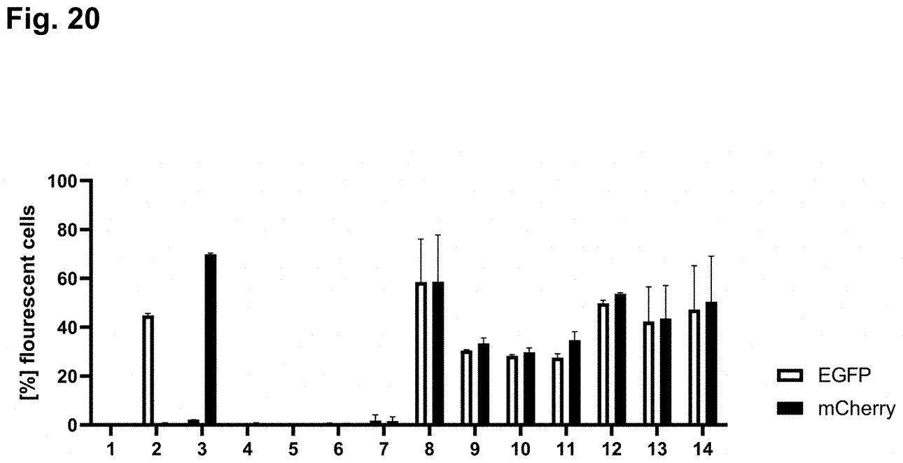

FIG. 20 shows the mean percentage of GFP and mCherry positive cells for each experimental condition 1 to 14 as outlined in Table 5 above. The respective standard deviations are indicated as error bars. As expected, hardly any fluorescent cells (<2%) were detected when cells had been transfected with mCherry_EGFP_pre rec alone (condition 7), i.e. without recombinase, whereas about 60% of cells were mCherry and EGFP positive when cells had been transfected with mCherry_EGFP_post rec (condition 8). This indicates that the mCherry and EGFP genes are inactive in the pre-recombination configuration in the absence of recombinase and active in the post-recombination configuration.

When FLPo expression plasmid was co-transfected together with mCherry_EGFPpre rec plasmid, the percentage of EGFP and mCherry positive cells increased to about 30% (conditions 9, 10 and 11) indicating successful RMCI and double gene activation. Co-transfection of FLPo expression plasmid with mCherry_EGFP_post rec had no impact on the expression of EGFP and mCherry (condition 8 vs. conditions 12, 13 and 14), showing that cassette inversion is inhibited in the post-recombination configuration. No fluorescent cells were detected when FLPo expression plasmid was transfected alone.

1. A double stranded DNA element comprising a coding strand and a template strand,
characterized in that
the coding strand comprises in 5′- to 3′-orientation in the following order
a first promoter,
a first recombinase recognition sequence comprising a mutation in the left inverted repeat,
a second promoter that is inverted with respect to the coding strand,
a first polyadenylation signal sequence and/or transcription termination element that is/are inverted with respect to the coding strand,
a first open reading frame that is inverted with respect to the coding strand and that is operably linked to the first polyadenylation signal sequence and/or transcription termination element,
a second recombinase recognition sequence comprising a mutation in the right inverted repeat and in inverted orientation with respect to the first recombinase recognition sequence,
a second open reading frame, and
a second polyadenylation signal sequence and/or transcription termination element operably linked to the second open reading frame.

2. A double stranded DNA element comprising a coding strand and a template strand, wherein the coding strand comprises in 5′- to 3′-orientation in the following order
a first promoter,
a first recombinase recognition sequence comprising a mutation in the left inverted repeat,
a Rep/Cap open reading frames including further promoters for the expression of the Rep and Cap proteins, which is inverted with respect to the coding strand,
a second recombinase recognition sequence comprising a mutation in the right inverted repeat and in inverted orientation with respect to the first recombinase recognition sequence, and
a polyadenylation signal sequence.

3. A double stranded DNA element comprising a coding strand and a template strand,
a) wherein the coding strand comprises in 5′- to 3′-orientation in the following order
a first promoter,
a first recombinase recognition sequence comprising a mutation in the left inverted repeat,
a second promoter that is inverted with respect to the coding strand,
a first polyadenylation signal sequence and/or transcription termination element that is inverted with respect to the coding strand,
a coding sequence,
which encodes either exclusively the Rep78 protein or exclusively the Rep68 protein, but not both, wherein
(i) optionally the internal P40 promoter is inactivated, and/or
(ii) the start codon of Rep52/40 is mutated into a non-start codon, and/or
(iii) splice donor and acceptor sites are removed,
which is inverted with respect to the coding strand, and
which is operably linked to the a first polyadenylation signal sequence and/or transcription termination element,
a second recombinase recognition sequence, which comprises a mutation in the right inverted repeat, and which is in inverted orientation with respect to the first recombinase recognition sequence, and
a Rep52/Rep40 and Cap open reading frames including a polyadenylation signal operably linked to said open reading frames,
or
b) wherein the coding strand comprises in 5′- to 3′-orientation in the following order
a first promoter,
a first recombinase recognition sequence comprising a mutation in the left inverted repeat,
a second promoter that is inverted with respect to the coding strand,
a first polyadenylation signal sequence and/or transcription termination element in that is inverted with respect to the coding strand,
a coding sequence,
which encodes either exclusively the Rep78 protein or exclusively the Rep68 protein, but not both, wherein
(i) optionally the internal promoter is inactivated, and/or
(ii) the start codon of the Rep52/40 open reading frame is mutated into a non-start codon, and
(iii) splice donor and acceptor sites are removed,
which is inverted with respect to the coding strand, and
which is operably linked to the first polyadenylation signal sequence and/or transcription termination element,
a second recombinase recognition sequence, which comprises a mutation in the right inverted repeat, and which is in inverted orientation with respect to the first recombinase recognition sequence, and
the Rep52 open reading frame, optionally with splice donor and acceptor sites removed, or the Rep40 open reading frame including a polyadenylation signal operably linked to said open reading frame.

4. The double stranded DNA element according to claim 2, wherein the first promoter is the P5 promoter.

5. The double stranded DNA element according to claim 4, wherein the second promoter is the P19 promoter.

6. The double stranded DNA element according to claim 3, wherein in c) the coding strand further comprises at its 3′-end
a third promoter, a cap open reading frame and a polyadenylation signal sequence and/or terminator sequence, wherein all are operably linked.

7. A double stranded DNA molecule comprising
a) the E1A open reading frame and the E1B open reading frame; and/or
b) the E2A open reading frame and the E4orf6 open reading frame;
characterized in that
the first and second open reading frames of a) or/and b) are contained in a double stranded DNA element comprising a coding strand and a template strand,
wherein the coding strand comprises in 5′- to 3′-orientation in the following order
a first promoter,
a first recombinase recognition sequence comprising a mutation in the right inverted repeat,
a second promoter that is inverted with respect to the coding strand,
the first open reading frame of a) or b) that is inverted with respect to the coding strand,
a second recombinase recognition sequence comprising a mutation in the left inverted repeat and in inverted orientation to the first recombinase recognition sequence, and
the second open reading frame of a) or b).

8. (canceled)

9. The double stranded DNA element or the double stranded DNA according to claim 2,
whereby the incubation of the double stranded DNA element or molecule with a recombinase functional with said first and second recombinase recognition sequence results
in the inversion of the sequence between the first and the second recombinase recognition sequence, whereafter the first promoter is operably linked to the first open reading frame, and
in the generation of a recombinase recognition sequence between the first promoter and the first gene following recombination that is no longer functional with said recombinase.

10. The double stranded DNA element or the double stranded DNA according to claim 1,
whereby the incubation of the double stranded DNA element or molecule with a recombinase functional with said first and second recombinase recognition sequence results
in the inversion of the sequence between the first and the second recombinase recognition sequence, whereafter the first promoter is operably linked to the first open reading frame and the second promoter is operably linked to the second open reading frame, and
in the generation of a recombinase recognition sequence between the first promoter and the first gene following recombination that is no longer functional with said recombinase.

11. A mammalian cell comprising
one or more double stranded DNA elements according to claim 1, or
at least one double stranded DNA element according to claim 2, or
one double stranded DNA molecule according to claim 2 and one double stranded DNA molecule according to claim 7, or
at least one double stranded DNA molecule according to claim 7, or
one or more double stranded DNAs according to claim 8.
